(19) United States
(12) Patent Application Publication
(10) Pub. No.: US 2005/0202973 A1
(43) Pub. Date: Sep. 15, 2005

(54) Aryl-alkyne compounds as herbicides

(76) Inventors: Jurgen Schaetzer, Basel (CH); Marian Valentiny, Bratislava (SK); Gerald Wayne Craig, Basel (CH); Jean Wenger, Basel (CH); Kurt Nebel, Basel (CH); Andre Stoller, Basel (CH); Roger Graham Hall, Basel (CH)

Correspondence Address: SYNGENTA CROP PROTECTION , INC.;PATENT AND TRADEMARK DEPARTMENT, 410 SWING ROAD, GREENSBORO, NC, 27409, US

(21) Appl. No.: 10/510,223

(22) PCT Filed: Apr. 2, 2003
(86) PCT No.: PCT/EP03/03467

§ 371 Date: Apr. 29, 2005

(30) Foreign Application Priority Data
Apr. 3, 2002 (CH) 558/02

(51) Int. Cl. A01N 43/46; A01N 43/60; C07D 43/02

(52) U.S. Cl. 504/227; 544/405; 544/209; 544/310; 544/180; 504/235

(57) ABSTRACT

Compounds of formula (I) wherein Q is a group formula (II), (III); Z is ═N—, formula (IV) or ═C(R₂)—; n is 0, 1, 2 or 3; R₃ or R₄ are each independently of the other hydrogen, alogen, —CN, C₁-C₄alkyl or C₁-C₄alkoxyl; or R₃ and R₄ together are C₂-C₅alkylene; and the agrochemically acceptable salts and all stereoisomers and tautomers of the compounds of formula (I) are suitable for use as herbicides.

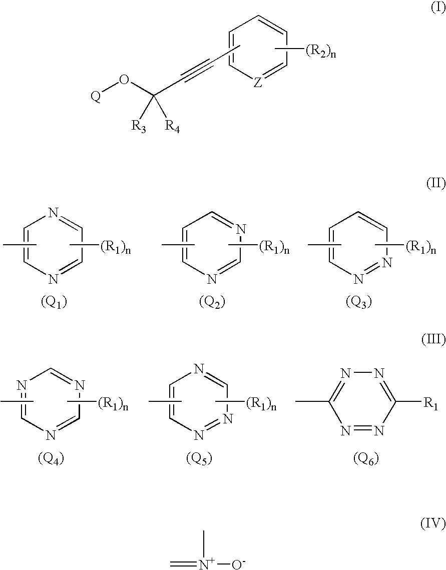

The present invention relates to novel, herbicidally active phenyl- and pyridyl-alkynes, to processes for their preparation, to compositions comprising those compounds, and to their use in controlling weeds, especially in crops of useful plants, or in inhibiting plant growth.

Phenylalkynes having herbicidal action are described, for example, in JP-A-1 1147 866, WO 01/55066 and WO 02/28182.

Novel phenyl- and pyridyl-alkynes that have herbicidal and growth-inhibiting properties have now been found.

The present invention accordingly relates to compounds of formula I 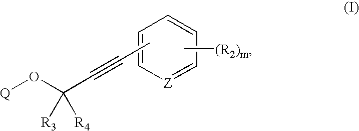
wherein
Q is a group 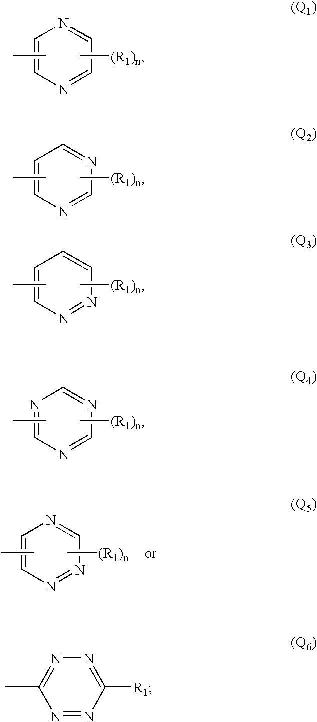
Z is ═N—, 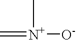
or ═C(R₂)—;
n is 0, 1, 2 or 3;
each R, independently is halogen, —CN, —SCN, —SF₅, —NO₂, —NR₅R₆, —CO₂R₇, —CONR₈NR₉, —C(R₁₀)═NOR₁₁, —COR₁₂, —OR₁₃, —SR₁₄, —SOR₁₅, —SO₂R₁₆, —OSO₂R₁₇, C₁-C₈alkyl, C₂-C₈alkenyl, C₂-C₈alkynyl or C₃-C₆cycloalkyl; or is C₁-C₈alkyl, C₂-C₈alkenyl or C₂-C₈alkynyl mono- to penta-substituted by halogen or mono-, di- or tri-substituted by —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —OR₂₉, —SR₃₀, —SOR₃₁, —SO₂R₃₂ or by C₃-C₆cycloalkyl; or
each R₁ independently is C₃-C₆cycloalkyl mono- to penta-substituted by halogen or mono-, di- or tri-substituted by —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —SR₃₀, —SOR₃₁, —SO₂R₃₂ or by C₃-C₆cycloalkyl; or
each R₁ independently is phenyl which may in turn be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or,
when Q is a group Q₁, Q₂, Q₃ or Q₅, two adjacent R₁ substituents may together form a C₁-C₇alkylene bridge which may be interrupted by from 1 to 3 hetero atoms selected from oxygen, nitrogen and sulfur and which may be mono- to penta-substituted by halogen or by C₁-C₆alkyl or mono-, di- or tri-substituted by C₁-C₆alkoxy, the total number of ring atoms being at least 5 and at most 9; or,
when Q is a group Q₁, Q₂, Q₃ or Q₅, two adjacent R₁ substituents may together form a C₂-C₇alkenylene bridge which may be interrupted by from 1 to 3 hetero atoms selected from oxygen, nitrogen and sulfur and which may be mono- to penta-substituted by halogen or by C₁-C₆alkyl or mono-, di- or tri-substituted by C₁-C₆alkoxy, the total number of ring atoms being at least 5 and at most 9;
R₃ or R₄ are each independently of the other hydrogen, halogen, —CN, C₁-C₄alkyl or C₁-C₄alkoxy; or
R₃ and R₄ together are C₂-C₅alkylene;
R₅ is hydrogen or C₁-C₈alkyl;
R₆ is hydrogen, C₁-C₈alkyl, C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₅ and R₆ together are a C₂-C₅alkylene chain which may be interrupted by an oxygen or sulfur atom;
R₇ is hydrogen, C₁-C₈alkyl, C₃-C₈alkenyl or C₃-C₈alkynyl, or is C₁-C₈alkyl, C₃-C₈alkenyl or C₃-C₈alkynyl mono- to penta-substituted by halogen or mono-, di- or tri-substituted by C₁-C₄alkoxy or by phenyl, it being possible for phenyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₈ is hydrogen or C₁-C₈alkyl;
R₉ is hydrogen or C₁-C₈alkyl, or is C₁-C₈alkyl mono-, di- or tri-substituted by —COOH, C₁-C₈alkoxycarbonyl or by —CN, or
R₉ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₈ and R₉ together are C₂-C₅alkylene;
R₁₀ is hydrogen, C₁-C₄alkyl, C₁-C₄haloalkyl or C₃-C₆cycloalkyl;
R₁₁ is hydrogen, C₁-C₈alkyl, C₃-C₈alkenyl, C₃-C₈alkynyl, C₁-C₄haloalkyl or C₃-C₆haloalkenyl;
R₁₂ is hydrogen, C₁-C₄alkyl, C₁-C₄haloalkyl or C₃-C₆cycloalkyl;
R₁₃ is hydrogen, C₁-C₈alkyl, C₃-C₈alkenyl or C₃-C₈alkynyl; or
R₁₃ is phenyl or phenyl-C₁-C₆alkyl, it being possible for the phenyl ring in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₈alkylthio, C₁-C₈alkylsulfinyl or by C₁-C₈alkylsulfonyl, or
R₁₃ is C₁-C₈alkyl mono- to penta-substituted by halogen or mono-, di- or tri-substituted by —CN, C₁-C₆alkylamino, di(C₁-C₆alkyl)amino or by C₁-C₄alkoxy;
R₁₄ is hydrogen, C₁-C₈alkyl, C₃-C₈alkenyl or C₃-C₈alkynyl, or is C₁-C₈alkyl mono- to penta-substituted by halogen or mono-, di- or tri-substituted by —CN or by C₁-C₄alkoxy;
R₁₅, R₁₆ and R₁₇ are each independently of the others C₁-C₈alkyl, C₃-C₈alkenyl or C₃-C₈alkynyl, or C₁-C₈alkyl mono- to penta-substituted by halogen or mono-, di- or tri-substituted by —CN or by C₁-C₄alkoxy;
R₁₈ is hydrogen or C₁-C₈alkyl;
R₁₉ is hydrogen, C₁-C₈alkyl, C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₈ and R₁₉ together are a C₂-C₆alkylene chain which may be interrupted by an oxygen or sulfur atom;
R₂₀ is hydrogen, C₁-C₈alkyl, C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₂₁ is hydrogen or C₁-C₈alkyl;
R₂₂ is hydrogen or C₁-C₈alkyl, or is C₁-C₈alkyl mono-, di- or tri-substituted by —COOH, C₁-C₈alkoxycarbonyl or by —CN, or
R₂₂ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₂₁ and R₂₂ together are C₂-C₅alkylene;
R₂₃ is hydrogen, C₁-C₄alkyl, C₁-C₄haloalkyl or C₃-C₆Cycloalkyl;
R₂₄ is hydrogen, C₁-C₄alkyl, C₁-C₄haloalkyl or C₃-C₆cycloalkyl;
R₂₅ is hydrogen, C₁-C₈alkyl, C₃-C₈alkenyl, C₃-C₈alkynyl, C₁-C₄haloalkyl or C₃-C₆haloalkenyl;
R₂₆ is hydrogen or C₁-C₈alkyl;
R₂₇ is hydrogen or C₁-C₈alkyl, or is C₁-C₈alkyl mono-, di- or tri-substituted by —COOH, C₁-C₈-alkoxycarbonyl or by —CN, or
R₂₇ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₂₆ and R₂₇ together are C₂-C₅alkylene;
R₂₈ is hydrogen or C₁-C₈alkyl;
R₂₉ and R₃₀ are each independently of the other hydrogen, C₁-C₈alkyl, C₃-C₈alkenyl or C₃-C₈alkynyl, or C₁-C₈alkyl mono- to penta-substituted by halogen or mono-, di- or tri-substituted by —CN or by C₁-C₄alkoxy;
R₃₁ and R₃₂ are each independently of the other C₁-C₈alkyl, C₃-C₈alkenyl or C₃-C₈alkynyl, or C₁-C₈alkyl mono- to penta-substituted by halogen or mono-, di- or tri-substituted by —CN or by C₁-C₄alkoxy;
m is 0, 1, 2, 3 or 4;
each R₂ independently is halogen, —CN, —SCN, —OCN, —N₃, —SF₅, —NO₂, —NR₃₃R₃₄, —CO₂R₃₅, —CONR₃₆R₃₇, —C(R₃₈)═NOR₃₉, —COR₄₀, —OR₄₁, —SR₄₂, —SOR₄₃, —SO₂R₄₄, —OSO₂R₄₅, —N([CO]ₚR₄₆)COR₄₇, —N(ORO₅₄)COR₅₅, —N(R₅₆)SO₂R₅₇, —N(SO₂R₅₈)SO₂R₅₉, —N═C(OR₆₀)R₆₁, —CR₆₂(OR₆₃)OR₆₄, —OC(O)NR₆₅R₆₆, —SC(O)NR₆₇R₆₈, —OC(S)NR₆₉R₇₀ or —N-phthalimide; or
R₂ is a 5- to 7-membered heterocyclic ring system which may be aromatic or partially or fully saturated and may contain from 1 to 4 hetero atoms selected from nitrogen, oxygen and sulfur, it being possible for that heterocyclic ring system in turn to be mono- to penta-substituted by halogen or by C₁-C₄alkyl or mono-, di- or tri-substituted by C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or by C₁-C₆alkylsulfonyl;
R₃₃ is hydrogen or C₁-C₆alkyl; and
R₃₄ is hydrogen, C₁-C₈alkyl, C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₃₃ and R₃₄ together are a C₂-C₅alkylene chain which may be interrupted by an oxygen or sulfur atom;
R₃₅ is hydrogen, C₁-C₈alkyl, C₃-C₈alkenyl or C₃-C₈alkynyl, or is C₁-C₈alkyl, C₃-C₈alkenyl or C₃-C₈alkynyl mono- to penta-substituted by halogen or mono-, di- or tri-substituted by C₁-C₄alkoxy or by phenyl, it being possible for phenyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₃₆ is hydrogen or C₁-C₈alkyl;
R₃₇ is hydrogen or C₁-C₈alkyl, or is C₁-C₈alkyl mono-, di- or tri-substituted by —COOH, C₁-C₈-alkoxycarbonyl or by —CN, or
R₃₇ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₃₆ and R₃₇ together are C₃-C₅alkylene;
R₃₈ is hydrogen, C₁-C₄alkyl, C₁-C₄haloalkyl or C₃-C₆cycloalkyl;
R₃₉ is hydrogen, C₁-C₈alkyl, C₃-C₈alkenyl, C₃-C₈alkynyl, C₁-C₄haloalkyl or C₃-C₆haloalkenyl;
R₄₀ is hydrogen, C₁-C₄alkyl, C₁-C₄haloalkyl, C₁-C₈alkylthio, —C(O)—C(O)OC₁-C₄alkyl or C₃-C₆-cycloalkyl;
R₄₁ is hydrogen, C₁-C₈alkyl, C₃-C₈alkenyl, C₃-C₈alkynyl, C₁-C₆alkoxy-C₁-C₆alkyl, C₁-C₈alkylcarbonyl, C₁-C₈alkoxycarbonyl, C₃-C₈alkenyloxycarbonyl, C₁-C₆alkoxy-C₁-C₆alkoxycarbonyl, C₁-C₆alkylthio-C₁-C₆alkyl, C₁-C₆alkylsulfinyl-C₁-C₆alkyl or C₁-C₆alkylsulfonyl-C₁-C₆alkyl; or
R₄₁ is phenyl or phenyl-C₁-C₆alkyl, it being possible for the phenyl ring in turn to be mono- to penta-substituted by halogen or by C₁-C₄alkyl or mono-, di- or tri-substituted by C₁-C₄haloalkyl, C₁-C₄alkoxy, —CN, —NO₂ or by —S(O)₂C₁-C₆alkyl, or
R₄₁ is C₁-C₈alkyl mono- to penta-substituted by halogen or mono-, di- or tri-substituted by —COOH, C₁-C₈alkoxycarbonyl, C₁-C₆alkylamino, di(C₁-C₆alkyl)amino or by —CN;
R₄₂ is hydrogen, C₁-C₈alkyl, C₃-C₈alkenyl or C₃-C₈alkynyl, or is C₁-C₈alkyl mono- to penta-substituted by halogen or mono-, di- or tri-substituted by —CN or by C₁-C₄alkoxy;
R₄₃ and R₄₄ are each independently of the other C₁-C₈alkyl, C₃-C₈alkenyl or C₃-C₈alkynyl, or C₁-C₈alkyl mono- to penta-substituted by halogen or mono-, di- or tri-substituted by —CN or by C₁-C₄alkoxy;
R₄₅ is C₁-C₈alkyl, C₁-C₈alkyl mono- to penta-substituted by halogen or mono-, di- or tri-substituted by —CN or by C₁-C₄alkoxy, or is C₃-C₈alkenyl or C₃-C₈alkynyl, or
R₄₅ is phenyl, it being possible for the phenyl ring to be mono- to penta-substituted by halogen or by C₁-C₄alkyl or mono-, di- or tri-substituted by C₁-C₄haloalkyl, C₁-C₄alkoxy, —CN, NO₂, C₁-C₈alkylthio, C₁-C₈alkylsulfinyl or by C₁-C₈alkylsulfonyl;
R₄₆ is hydrogen, C₁-C₈alkyl, C₃-C₈alkenyl, C₃-C₈alkynyl or C₁-C₄haloalkyl;
R₄₇ is hydrogen, C₁-C₈alkyl, C₁-C₄alkoxy, C₃-C₈alkenyl or C₃-C₈alkynyl, or is C₁-C₈alkyl mono- to penta-substituted by halogen or mono-, di- or tri-substituted by —CN, C₁-C₄alkoxy, C₁-C₈alkoxycarbonyl, —NH₂, C₁-C₄alkylamino, di(C₁-C₄alkyl)amino, —NR₄₈COR₄₉, —NR₅₀SO₂R₅₁, or by —NR₅₂CO₂R₅₃, or
R₄₇ is phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
p is 0 or 1;
R₄₈, R₄₉, R₅₀, R₅₁, R₅₂ and R₅₃ are each independently of the others hydrogen, C₁-C₈alkyl, phenyl, benzyl or naphthyl, it being possible for the three last-mentioned aromatic radicals in turn to be mono- to penta-substituted by halogen or by C₁-C₈alkyl or mono-, di- or tri-substituted by C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₅₄ and R₅₅ are each independently of the other hydrogen, C₁-C₈alkyl, or phenyl which may in turn be mono- to penta-substituted by halogen or by C₁-C₄alkyl or mono-, di- or tri-substituted by C₁-C₄haloalkyl, C₁-C₄alkoxy, —CN, —NO₂, C₁-C₈alkylthio, C₁-C₈alkylsulfinyl or by C₁-C₈alkylsulfonyl;
R₅₆ is hydrogen, C₁-C₈alkyl, C₁-C₄haloalkyl, C₁-C₄alkoxy, C₃-C₈alkenyl, C₃-C₈alkynyl or benzyl, it being possible for benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₈alkylthio, C₁-C₈alkylsulfinyl or by C₁-C₈alkylsulfonyl;
R₅₇ is C₁-C₈alkyl, C₁-C₄haloalkyl, phenyl, benzyl or naphthyl, it being possible for the last three aromatic rings to be mono- to penta-substituted by halogen or by C₁-C₄alkyl or mono-, di- or tri-substituted by C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₅₈ and R₅₉ are each independently of the other C₁-C₈alkyl, C₃-C₈alkenyl, C₃-C₈alkynyl, phenyl, benzyl or naphthyl, it being possible for the last three aromatic rings to be mono- to penta-substituted by halogen or by C₁-C₄alkyl or mono-, di- or tri-substituted by C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₆₀ and R₆₁ are each independently of the other hydrogen or C₁-C₆alkyl;
R₆₂, R₆₃ and R₆₄ are each independently of the others hydrogen or C₁-C₈alkyl, or
R₆₃ and R₆₄ together form a C₂-C₅alkylene bridge;
R₆₅, R₆₆, R₆₇, R₆₈, R₆₉ and R₇₀ are each independently of the others hydrogen or C₁-C₈alkyl, or
R₆₅ and R₆₆, or R₆₇ and R₆₈, or R₆₉ and R₇₀ in each case together form a C₂-C₅alkylene bridge; or
each R₂ independently is C₁-C₈alkyl, or is C₁-C₈alkyl mono- to penta-substituted by halogen or mono-, di- or tri-substituted by —CN, —N₃, —SCN, —NO₂, —NR₇₁R₇₂, —CO₂R₇₃, —CONR₇₄R₇₅, —COR₇₆, —C(R₇₇)═NOR₇₈, —C(S)NR₇₉R₈₀, —C(C₁-C₄alkylthio)═NR₈₁, —OR₈₂, —SR₈₃, —SOR₈₄, —SO₂R₈₅, —O(SO₂)R₈₆, —N(R₈₇)CO₂R₈₈, —N(R₈₉)COR₉₀, —S⁺(R₉₁)₂, —N⁺(R₉₂)₃, —Si(R₉₃)₃ or by C₃-C₆cycloalkyl; or
each R₂ independently is C₁-C₈alkyl substituted by a 5- to 7-membered heterocyclic ring system which may be aromatic or partially or fully saturated and may contain from 1 to 4 hetero atoms selected from oxygen, nitrogen and sulfur, it being possible for that heterocyclic ring system in turn to be mono- to penta-substituted by halogen or by C₁-C₄alkyl or mono-, di- or tri-substituted by C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or by C₁-C₆alkylsulfonyl; or
each R₂ independently is C₂-C₈alkenyl, or is C₂-C₈alkenyl mono- to penta-substituted by halogen or mono-, di- or tri-substituted by —CN, —NO₂, —CO₂R₉₄, —CONR₉₅R₉₆, —COR₉₇, —C(R₉₈)═NOR₉₉, —C(S)NR₁₀₀R₁₀₁, —C(C₁-C₄alkylthio)═NR₁₀₂, —OR₁₀₃, —Si(R₁₀₄)₃ or by C₃-C₆-cycloalkyl; or
each R₂ independently is C₂-C₈alkynyl, or is C₂-C₈alkynyl mono- to penta-substituted by halogen or mono-, di- or tri-substituted by —CN, —CO₂R₁₀₅, —CONR₁₀₆R₁₀₇, —COR₁₀₈, —C(R₁₀₉)═NOR₁₁₀, —C(S)NR₁₁₁R₁₁₂, —C(C₁-C₄alkylthio)═NR₁₁₃, —OR₁₁₄, —Si(R₁₁₅)₃ or by C₃-C₆cycloalkyl; or
each R₂ independently is C₃-C₆cycloalkyl, or is C₃-C₆cycloalkyl mono- to penta-substituted by halogen or mono-, di- or tri-substituted by —CN, —CO₂R₁₁₆, —CONR₁₁₇R₁₁₈, —COR₁₁₉, —C(R₁₂₀)═NOR₁₂₁, —C(S)NR₁₂₂R₁₂₃ or by —C(C₁-C₄alkylthio)═NR₁₂₄; or
two adjacent R₂ substituents together form a C₁-C₇alkylene bridge which may be interrupted by from 1 to 3 hetero atoms selected from oxygen, nitrogen and sulfur and may be mono- to penta-substituted by halogen or by C₁-C₆alkyl or mono-, di- or tri-substituted by C₁-C₆alkoxy, the total number of ring atoms being at least 5 and at most 9; or
two adjacent R₂ substituents together form a C₂-C₇alkenylene bridge which may be interrupted by from 1 to 3 hetero atoms selected from oxygen, nitrogen and sulfur and may be mono- to penta-substituted by halogen or by C₁-C₆alkyl or mono-, di- or tri-substituted by C₁-C₆alkoxy, the total number of ring atoms being at least 5 and at most 9;
R₇₁ is hydrogen or C₁-C₈alkyl;
R₇₂ is hydrogen, C₁-C₈alkyl, C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₇₁ and R₇₂ together are a C₂-C₅alkylene chain which may be interrupted by an oxygen or sulfur atom;
R₇₃ is hydrogen, C₁-C₈alkyl, C₃-C₈alkenyl or C₃-C₈alkynyl, or is C₁-C₈alkyl, C₃-C₈alkenyl or C₃-C₈alkynyl mono- to penta-substituted by halogen or mono-, di- or tri-substituted by C₁-C₄alkoxy or by phenyl, it being possible for phenyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₇₄ is hydrogen or C₁-C₈alkyl;
R₇₅ is hydrogen, C₁-C₈alkyl or C₃-C₇cycloalkyl, or is C₁-C₈alkyl mono-, di- or tri-substituted by —COOH, C₁-C₈alkoxycarbonyl, C₁-C₆alkoxy or by —CN; or
R₇₅ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₇₄ and R₇₅ together are a C₂-C₅alkylene chain which may be interrupted by an oxygen or sulfur atom;
R₇₆ is hydrogen, C₁-C₄alkyl, C₁-C₄haloalkyl or C₃-C₆cycloalkyl;
R₇₇ is hydrogen, C₁-C₄alkyl, C₁-C₄haloalkyl or C₃-C₆cycloalkyl;
R₇₈ is hydrogen, C₁-C₈alkyl, C₃-C₈alkenyl, C₃-C₈alkynyl, C₁-C₄haloalkyl or C₃-C₆haloalkenyl; and
R₇₉ is hydrogen or C₁-C₈alkyl;
R₈₀ is hydrogen or C₁-C₈alkyl, or is C₁-C₈alkyl mono-, di- or tri-substituted by —COOH, C₁-C₈-alkoxycarbonyl or by —CN; or
R₈₀ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₇₉ and R₈₀ together are C₂-C₅alkylene;
R₈₁ is hydrogen or C₁-C₈alkyl;
R₈₂ is —Si(C₁-C₆alkyl)₃, C₃-C₈alkenyl or C₃-C₈alkynyl, or is C₁-C₈alkyl which is mono- to penta-substituted by halogen or mono-, di- or tri-substituted by —CN, —NH₂, C₁-C₆alkylamino, di(C₁-C₆alkyl)amino or by C₁-C₄alkoxy;
R₈₃ is hydrogen, C₁-C₈alkyl, C₃-C₈alkenyl or C₃-C₈alkynyl, or is C₁-C₈alkyl which is mono- to penta-substituted by halogen or mono-, di- or tri-substituted by —CN, —NH₂, C₁-C₆alkylamino, di(C₁-C₆alkyl)amino or by C₁-C₄alkoxy;
R₈₄, R₈₅ and R₈₆ are each independently of the others C₁-C₈alkyl, C₃-C₈alkenyl or C₃-C₈-alkynyl, or C₁-C₈alkyl which is mono- to penta-substituted by halogen or mono-, di- or tri-substituted by —CN or by C₁-C₄alkoxy;
R₈₇ and R₈₉ are each independently of the other hydrogen, C₁-C₈alkyl or C₁-C₈alkoxy;
R₈₈ is C₁-C₈alkyl;
R₉₀ is hydrogen or C₁-C₈alkyl;
R₉₁ is C₁-C₄alkyl;
R₉₂ and R₉₃ are each independently of the other C₁-C₆alkyl;
R₉₄ is hydrogen or is C₁-C₈alkyl, C₃-C₈alkenyl or C₃-C₈alkynyl, each of which may be mono- to penta-substituted by halogen or mono-, di- or tri-substituted by C₁-C₄alkoxy or by phenyl, it being possible for phenyl in turn to be mono- to penta-substituted by halogen or by C₁-C₄-alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₉₅ is hydrogen or C₁-C₈alkyl;
R₉₆ is hydrogen or C₁-C₈alkyl, or is C₁-C₈alkyl mono-, di- or tri-substituted by —COOH, C₁-C₈alkoxycarbonyl or by —CN; or
R₉₆ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₉₅ and R₉₆ together are C₂-C₅alkylene;
R₉₇ and R₉₈ are each independently of the other hydrogen, C₁-C₄alkyl, C₁-C₄haloalkyl or C₃-C₆cycloalkyl;
R₉₉ is hydrogen, C₁-C₈alkyl, C₃-C₈alkenyl, C₃-C₈alkynyl, C₁-C₄haloalkyl or C₃-C₆haloalkenyl;
R₁₀₀ is hydrogen or C₁-C₈alkyl;
R₁₀₁ is hydrogen or C₁-C₈alkyl, or is C₁-C₈alkyl mono-, di- or tri-substituted by —COOH, C₁-C₈-alkoxycarbonyl or by —CN; or
R₁₀₁ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₀₀ and R₁₀₁ together are C₂-C₅alkylene;
R₁₀₂ is hydrogen or C₁-C₈alkyl;
R₁₀₃ is hydrogen, C₁-C₈alkyl, —Si(C₁-C₆alkyl)₃, C₃-C₈alkenyl or C₃-C₈alkynyl;
R₁₀₄ is C₁-C₆alkyl;
R₁₀₅ is hydrogen or is C₁-C₈alkyl, C₃-C₈alkenyl or C₃-C₈alkynyl, each of which may be mono- to penta-substituted by halogen or mono-, di- or tri-substituted by C₁-C₄alkoxy or by phenyl, it being possible for phenyl in turn to be mono- to penta-substituted by halogen or by C₁-C₄-alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₁₀₆ is hydrogen or C₁-C₈alkyl;
R₁₀₇ is hydrogen or C₁-C₈alkyl, or is C₁-C₈alkyl mono-, di- or tri-substituted by —COOH, C₁-C₈alkoxycarbonyl or by —CN, or
R₁₀₇ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₀₆ and R₁₀₇ together are C₂-C₅alkylene;
R₁₀₈ is hydrogen, C₁-C₄alkyl, C₁-C₄haloalkyl or C₃-C₆cycloalkyl;
R₁₀₉ is hydrogen, C₁-C₄alkyl, C₁-C₄haloalkyl or C₃-C₆cycloalkyl;
R₁₁₀ is hydrogen, C₁-C₈alkyl, C₃-C₈alkenyl, C₃-C₈alkynyl, C₁-C₄haloalkyl or C₃-C₆haloalkenyl;
R₁₁₁ is hydrogen or C₁-C₈alkyl;
R₁₁₂ is hydrogen or C₁-C₈alkyl, or is C₁-C₈alkyl mono-, di- or tri-substituted by —COOH, C₁-C₈alkoxycarbonyl or by —CN; or
R₁₁₂ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₁₁ and R₁₁₂ together are C₂-C₅alkylene;
R₁₁₃ is hydrogen or C₁-C₈alkyl;
R₁₁₄ is hydrogen, C₁-C₈alkyl, —Si(C₁-C₆alkyl)₃, C₃-C₈alkenyl or C₃-C₈alkynyl;
R₁₁₅ is C₁-C₆alkyl;
R₁₁₆ is hydrogen or is C₁-C₈alkyl, C₃-C₈alkenyl or C₃-C₈alkynyl, each of which may be mono- to penta-substituted by halogen or mono-, di- or tri-substituted by C₁-C₄alkoxy or by phenyl, it being possible for phenyl in turn to be mono- to penta-substituted by halogen or by C₁-C₄-alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₁₁₇ is hydrogen or C₁-C₈alkyl;
R₁₁₈ is hydrogen or C₁-C₈alkyl, or is C₁-C₈alkyl mono-, di- or tri-substituted by —COOH, C₁-C₈alkoxycarbonyl or by —CN; or
R₁₁₈ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₁₇ and R₁₁₈ together are C₂-C₅alkylene;
R₁₁₉ is hydrogen, C₁-C₄alkyl, C₁-C₄haloalkyl or C₃-C₆cycloalkyl;
R₁₂₀ is hydrogen, C₁-C₄alkyl, C₁-C₄haloalkyl or C₃-C₆cycloalkyl;
R₁₂₁ is hydrogen, C₁-C₈alkyl, C₃-C₈alkenyl, C₃-C₈alkynyl, C₁-C₄haloalkyl or C₃-C₆haloalkenyl;
R₁₂₂ is hydrogen or C₁-C₈alkyl;
R₁₂₃ is hydrogen or C₁-C₈alkyl, or is C₁-C₈alkyl mono-, di- or tri-substituted by —COOH, C₁-C₈alkoxycarbonyl or by —CN; or
R₁₂₃ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₂₂ and R₁₂₃ together are C₂-C₅alkylene; and
R₁₂₄ is hydrogen or C₁-C₈alkyl,
and to the agrochemically acceptable salts and all stereoisomers and tautomers of the compounds of formula I.

When n is 0, all free valencies on the heterocyclic groups Q₁ to Q₈ of the compounds of formula I are occupied by hydrogen. When m is 0, all free valencies on the phenyl or pyridyl ring of the compounds of formula I are occupied by hydrogen.

Examples of substituents that are formed as a result of R₅ and R₆ together or R₁₈ and R₁₉ together or R₃₆ and R₃₇ together or R₇₄ and R₇₅ together being a C₂-C₅alkylene chain which may be interrupted by an oxygen or sulfur atom are piperidine, morpholine, thiomorpholine and pyrrolidine.

Examples of heterocyclic ring systems which may be aromatic or partially or fully saturated in the definition of R₂ are:

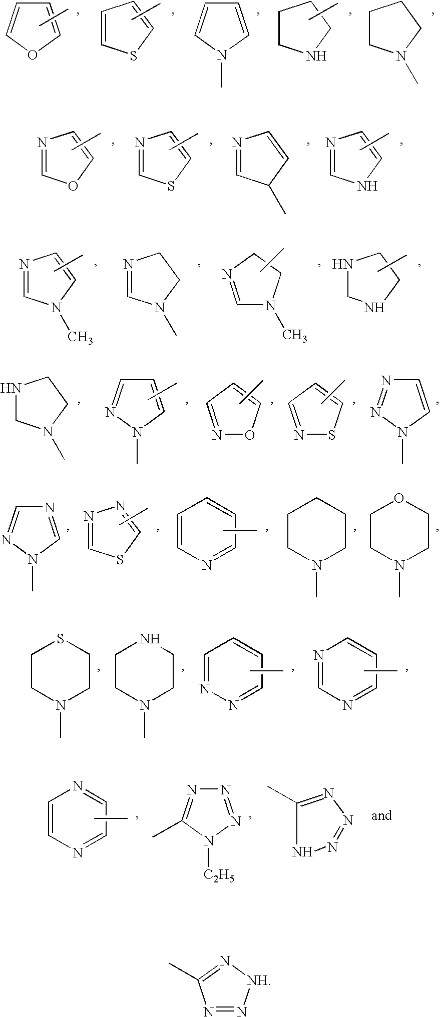

The alkyl groups appearing in the substituent definitions may be straight-chained or branched and are, for example, methyl, ethyl, n-propyl, isopropyl, n-butyl, sec-butyl, isobutyl, tert-butyl and the pentyl, hexyl, heptyl and octyl isomers.

Halogen is fluorine, chlorine, bromine or iodine, preferably fluorine or chlorine.

Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl or 2,2,2-trichloroethyl; preferably trichloromethyl, difluorochloromethyl, difluoromethyl, trifuoromethyl or dichlorofluoromethyl.

Alkoxy groups have a chain length of preferably from 1 to 6, especially from 1 to 4, carbon atoms. Alkoxy is, for example, methoxy, ethoxy, propoxy, isopropoxy, n-butoxy, isobutoxy, sec-butoxy or tert-butoxy, or the pentyloxy and hexyloxy isomers; preferably methoxy or ethoxy.

Alkoxy, alkenyl, alkynyl, alkoxyalkyl, alkylthio, alkylsulfonyl, alkylsulfinyl, alkylaminoalkoxy, alkoxycarbonyl, alkylcarbonyloxy, alkenylthio, alkenylsulfonyl, alkenylsulfinyl, alkynylsulfonyl, alkynylthio and alkynylsulfinyl groups are derived from the mentioned alkyl groups. The alkenyl and alkynyl groups may be mono- or poly-unsaturated. Alkenyl is, for example, vinyl, allyl, methallyl, 1-methylvinyl or but-2-en-1-yl. Alkynyl is, for example, ethynyl, propargyl, but-2-yn-1-yl, 2-methylbutyn-2-yl or but-3-yn-2-yl.

Alkylthio groups preferably have a chain length of from 1 to 4 carbon atoms. Alkylthio is, for example, methylthio, ethylthio, propylthio, isopropylthio, n-butylthio, isobutylthio, sec-butylthio or tert-butylthio, preferably methylthio or ethylthio. Alkylsulfinyl is, for example, methylsulfinyl, ethylsulfinyl, propylsulfinyl, isopropylsulfinyl, n-butylsulfinyl, isobutylsulfinyl, sec-butylsulfinyl or tert-butylsulfinyl; preferably methylsulfinyl or ethylsulfinyl. Alkylsulfonyl is, for example, methylsulfonyl, ethylsulfonyl, propylsulfonyl, isopropylsulfonyl, n-butylsulfonyl, isobutylsulfonyl, sec-butylsulfonyl or tert-butylsulfonyl; preferably methylsulfonyl or ethylsulfonyl.

Alkoxyalkyl groups preferably have from 1 to 6 carbon atoms. Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

Substituents where two adjacent R₁ substituents (on the group Q₁, Q₂, Q₃ or Q₅) together form a C₁-C₇alkylene bridge which may be interrupted by from 1 to 3 hetero atoms selected from oxygen, nitrogen and sulfur and may be mono- to penta-substituted by halogen or by C₁-C₆alkyl or mono-, di- or tri-substituted by C₁-C₆alkoxy, the total number of ring atoms being at least 5 and at most 9, or where two adjacent R₁ substituents together form a C₂-C₇alkenylene bridge which may be interrupted by from 1 to 3 hetero atoms selected from oxygen, nitrogen and sulfur and may be mono- to penta-substituted by halogen or by C₁-C₆alkyl or mono-, di- or tri-substituted by C₁-C₆alkoxy, the total number of ring atoms being at least 5 and at most 9, have, for example, the following structures:

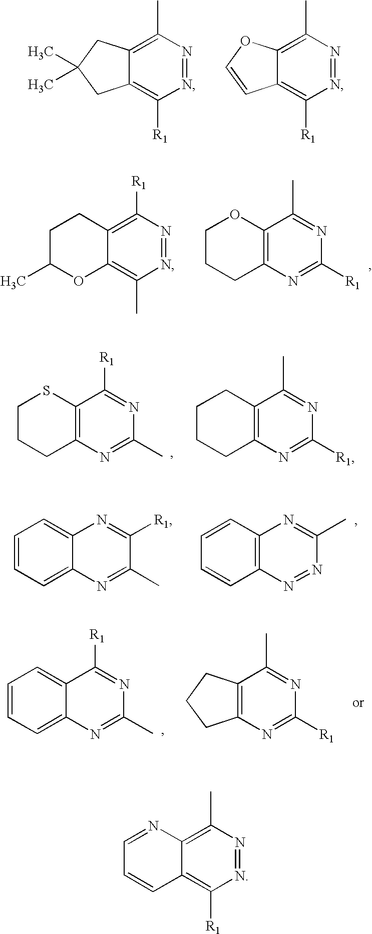

Substituents where two adjacent R₂ substituents together form a C₁-C₇alkylene bridge which may be interrupted by from 1 to 3 hetero atoms selected from oxygen, nitrogen and sulfur and may be mono- to penta-substituted by halogen or by C₁-C₆alkyl or mono-, di- or tri-substituted by C₁-C₆alkoxy, the total number of ring atoms being at least 5 and at most 9, or where two adjacent R₂ substituents together form a C₂-C₇alkenylene bridge which may be interrupted by from 1 to 3 hetero atoms selected from oxygen, nitrogen and sulfur and may be mono- to penta-substituted by halogen or by C₁-C₆alkyl or mono-, di- or tri-substituted by C₁-C₆alkoxy, the total number of ring atoms being at least 5 and at most 9, have, for example, the following structures:

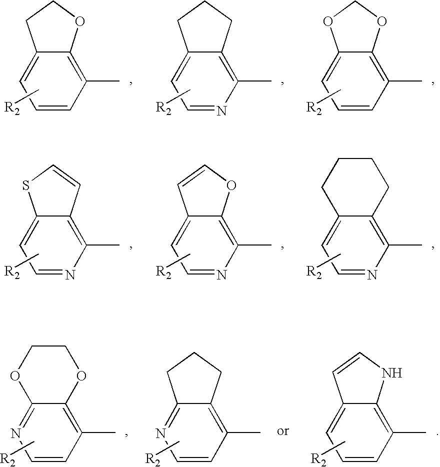

The invention relates also to the salts which the compounds of formula I are able to form preferably with amines, alkali metal and alkaline earth metal bases or quaternary ammonium bases. Suitable salt formers are described, for example, in WO 98/41089.

Among the alkali metal and alkaline earth metal hydroxides as salt formers, special mention should be made of the hydroxides of lithium, sodium, potassium, magnesium and calcium, especially the hydroxides of sodium and potassium.

Examples of amines suitable for ammonium salt formation include ammonia as well as primary, secondary and tertiary C₁-C₁₈alkylamines, C₁-C₄hydroxyalkylamines and C₂-C₄alkoxyalkylamines, for example methylamine, ethylamine, n-propylamine, isopropylamine, the four butylamine isomers, n-amylamine, isoamylamine, hexylamine, heptylamine, octylamine, nonylamine, decylamine, pentadecylamine, hexadecylamine, heptadecylamine, octadecylamine, methyl-ethylamine, methyl-isopropylamine, methyl-hexylamine, methyl-nonylamine, methyl-pentadecylamine, methyl-octadecylamine, ethyl-butylamine, ethyl-heptylamine, ethyl-octylamine, hexyl-heptylamine, hexyl-octylamine, dimethylamine, diethylamine, di-n-propylamine, diisopropylamine, di-n-butylamine, di-n-amylamine, diisoamylamine, dihexylamine, diheptylamine, dioctylamine, ethanolamine, n-propanolamine, isopropanolamine, N,N-diethanolamine, N-ethylpropanolamine, N-butylethanolamine, allylamine, n-butenyl-2-amine, n-pentenyl-2-amine, 2,3-dimethylbutenyl-2-amine, dibutenyl-2-amine, n-hexenyl-2-amine, propylenediamine, trimethylamine, triethylamine, tri-n-propylamine, triisopropylamine, tri-n-butylamine, triisobutylamine, tri-sec-butylamine, tri-n-amylamine, methoxyethylamine and ethoxyethylamine; hetero-cyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylenediamines, benzidines, naphthylamines and o-, m- and p-chloroanilines; but especially triethylamine, isopropylamine and diisopropylamine.

Preferred quaternary ammonium bases suitable for salt formation correspond, for example, to the formula [N(RₐR_{b}R_{c}R_{d})]OH wherein Rₐ, R_{b}, R_{c} and R_{d} are each independently of the others C₁-C₄alkyl. Other suitable tetraalkylammonium bases with other anions can be obtained, for example, by anion exchange reactions.

Preference is given to compounds of formula I wherein each R₁ independently is halogen, —CN, —SCN, —SF₅, —NO₂, —NR₅R₆, —CO₂R₇, —CONR₈R₉, —C(R₁₀)═NOR₁₁, —COR₁₂, —OR₁₃, —SR₁₄, —SOR₁₅, —SO₂R₁₆, —OSO₂R₁₇, C₁-C₈alkyl, C₂-C₈alkenyl, C₂-C₈alkynyl or C₃-C₆cycloalkyl; or is C₁-C₈alkyl, C₂-C₈alkenyl or C₂-C₈alkynyl mono-, di- or tri-substituted by halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —OR₂₉, —SR₃₀, —SOR₃₁, —SO₂R₃₂ or by C₃-C₆cycloalkyl; or
each R₁ independently is C₃-C₆cycloalkyl mono-, di- or tri-substituted by halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —SR₃₀, —SOR₃₁, —SO₂R₃₂ or by C₃-C₆cycloalkyl; or
each R₁ independently is phenyl which may in turn be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or,
when Q is a group Q₁, Q₂, Q₃ or Q₅, two adjacent R₁ substituents together may form a C₁-C₇alkylene bridge which may be interrupted by from 1 to 3 hetero atoms selected from oxygen, nitrogen and sulfur and may be mono-, di- or tri-substituted by halogen, C₁-C₆alkyl or by C₁-C₆alkoxy, the total number of ring atoms being at least 5 and at most 9; or,
when Q is a group Q₁, Q₂, Q₃ or Q₅, two adjacent R₁ substituents together may form a C₂-C₇alkenylene bridge which may be interrupted by from 1 to 3 hetero atoms selected from oxygen, nitrogen and sulfur and may be mono-, di- or tri-substituted by halogen, C₁-C₆alkyl or by C₁-C₆alkoxy, the total number of ring atoms being at least 5 and at most 9;
R₃ or R₄ are each independently of the other hydrogen, halogen, —CN, C₁-C₄alkyl or C₁-C₄alkoxy; or
R₃ and R₄ together are C₂-C₅alkylene;
R₅ is hydrogen or C₁-C₈alkyl;
R₆ is hydrogen, C₁-C₈alkyl, C₃-C₈alkenyl, C₃-C₈alkynyl, phenyl or bent, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₅ and R₆ together are a C₂-C₅alkylene chain which may be interrupted by an oxygen or sulfur atom;
R₇ is hydrogen, C₁-C₈alkyl, C₃-C₈alkenyl or C₃-C₈alkynyl, or is C₁-C₈alkyl, C₃-C₈alkenyl or C₃-C₈alkynyl mono-, di- or tri-substituted by halogen, C₁-C₄alkoxy or by phenyl, it being possible for phenyl in turn to be mono-, di- or tri-substituted by halogen, C₁-C₄alkyl, C₁-C₄-haloalkyl, C₁-C₄alkoxy, —CN, —NO₂, C₁-C₄alkylthio, C₁-C₄alkylsulfinyl or by C₁-C₄alkylsulfonyl;
R₈ is hydrogen or C₁-C₈alkyl;
R₉ is hydrogen or C₁-C₈alkyl, or is C₁-C₈alkyl mono-, di- or tri-substituted by —COOH, C₁-C₈alkoxycarbonyl or by —CN, or
R₉ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₈ and R₉ together are C₂-C₅alkylene;
R₁₀ is hydrogen, C₁-C₄alkyl, C₁-C₄haloalkyl or C₃-C₆cycloalkyl;
R₁₁ is hydrogen, C₁-C₈alkyl, C₃-C₈alkenyl, C₃-C₈alkynyl, C₁-C₄haloalkyl or C₃-C₆haloalkenyl;
R₁₂ is hydrogen, C₁-C₄alkyl, C₁-C₄haloalkyl or C₃-C₆cycloalkyl;
R₁₃ is hydrogen, C₁-C₈alkyl, C₃-C₈alkenyl or C₃-C₈alkynyl; or
R₁₃ is phenyl or phenyl-C₁-C₆alkyl, it being possible for the phenyl ring in turn to be mono-, di- or tri-substituted by halogen, C₁-C₄alkyl, C₁-C₄haloalkyl, C₁-C₄alkoxy, —CN, —NO₂, C₁-C₈-alkylthio, C₁-C₈alkylsulfinyl or by C₁-C₈alkylsulfonyl; or
R₁₃ is C₁-C₈alkyl mono-, di- or tri-substituted by halogen, —CN, C₁-C₆alkylamino, di(C₁-C₆-alkyl)amino or by C₁-C₄alkoxy;
R₁₄ is hydrogen, C₁-C₈alkyl, C₃-C₈alkenyl or C₃-C₈alkynyl, or is C₁-C₈alkyl mono-, di- or tri-substituted by halogen, —CN or by C₁-C₄alkoxy;
R₁₅, R₁₆ and R₁₇ are each independently of the others C₁-C₈alkyl, C₃-C₈alkenyl or C₃-C₈-alkynyl, or C₁-C₈alkyl mono-, di- or tri-substituted by halogen, —CN or by C₀-C₄alkoxy;
R₁₈ is hydrogen or C₁-C₈alkyl;
R₁₉ is hydrogen, C₁-C₈alkyl, C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₈ and R₁₉ together are a C₂-C₅alkylene chain which may be interrupted by an oxygen or sulfur atom;
R₂₀ is hydrogen, C₁-C₈alkyl, C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl;
R₂₁ is hydrogen or C₁-C₈alkyl;
R₂₂ is hydrogen or C₁-C₈alkyl, or is C₁-C₈alkyl mono-, di- or tri-substituted by —COOH, C₁-C₈alkoxycarbonyl or by —CN, or
R₂₂ is C₃-C₈alkenyl, C₃-C₈alkynyl phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₂₁ and R₂₂ together are C₂-C₅alkylene;
R₂₃ is hydrogen, C₁-C₄alkyl, C₁-C₄haloalkyl or C₃-C₆cycloalkyl;
R₂₄ is hydrogen, C₁-C₄alkyl, C₁-C₄haloalkyl or C₃-C₆cycloalkyl;
R₂₅ is hydrogen, C₁-C₈alkyl, C₃-C₈alkenyl, C₃-C₈alkynyl, C₁-C₄haloalkyl or C₃-C₆haloalkenyl;
R₂₆ is hydrogen or C₁-C₈alkyl;
R₂₇ is hydrogen or C₁-C₈alkyl, or is C₁-C₈alkyl mono-, di- or tri-substituted by —COOH, C₁-C₈alkoxycarbonyl or by —CN, or
R₂₇ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₂₆ and R₂₇ together are C₂-C₅alkylene;
R₂₈ is hydrogen or C₁-C₈alkyl;
R₂₉ and R₃₀ are each independently of the other hydrogen, C₁-C₈alkyl, C₃-C₈alkenyl or C₃-C₈alkynyl, or C₁-C₈alkyl mono-, di- or tri-substituted by halogen, —CN or by C₁-C₄alkoxy;
R₃₁ and R₃₂ are each independently of the other C₁-C₈alkyl, C₃-C₈alkenyl or C₃-C₈alkynyl, or C₁-C₈alkyl mono-, di- or tri-substituted by halogen, —CN or by C₁-C₄alkoxy;
m is 0, 1, 2, 3 or 4;
each R₂ independently is hydrogen, halogen, —CN, —SCN, —OCN, —N₃, —SF₅, —NO₂, —NR₃₃R₃₄, —CO₂R₃₅, —CONR₃₆R₃₇, —C(R₃₈)═NOR₃₉, —COR₄₀, —OR₄₁, —SR₄₂, —SOR₄₃, —SO₂R₄₄, —OSO₂R₄₅, —N([CO]ₚR₄₆)COR₄₇, —N(OR₅₄)COR₅₅, —N(R₅₆)SO₂R₅₇, —N(SO₂R₅₈)SO₂R₅₉, —N↑C(OR₆₀)R₆₁, —CR₆₂(OR₆₃)OR₆₄, —OC(O)NR₆₅R₆₆, —SC(O)NR₆₇R₆₈, —OC(S)NR₆₉R₇₀ or —N-phthalimide; or
R₂ is a 5- to 7-membered heterocyclic ring system which may be aromatic or partially or fully saturated and may contain from 1 to 4 hetero atoms selected from nitrogen, oxygen and sulfur, it being possible for that heterocyclic ring system in turn to be mono-, di- or tri-substituted by halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or by C₁-C₆alkylsulfonyl;
R₃₃ is hydrogen or C₁-C₈alkyl; and
R₃₄ is hydrogen, C₁-C₈alkyl, C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₃₃ and R₃₄ together are a C₂-C₅alkylene chain which may be interrupted by an oxygen or sulfur atom;
R₃₅ is hydrogen, C₁-C₈alkyl, C₃-C₈alkenyl or C₃-C₈alkynyl, or is C₁-C₈alkyl, C₃-C₈alkenyl or C₃-C₈alkynyl mono-, di- or tri-substituted by halogen, C₁-C₄alkoxy or by phenyl, it being possible for phenyl in turn to be mono-, di- or tri-substituted by halogen, C₁-C₄alkyl, C₁-C₄-haloalkyl, C₁-C₄alkoxy, —CN, —NO₂, C₁-C₄alkylthio, C₁-C₄alkylsulfinyl or by C₁-C₄alkylsulfonyl;
R₃₆ is hydrogen or C₁-C₈alkyl;
R₃₇ is hydrogen or C₁-C₈alkyl, or is C₁-C₈alkyl mono-, di- or tri-substituted by —COOH, C₁-C₈alkoxycarbonyl or by —CN, or
R₃₇ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₃₆ and R₃₇ together are C₃-C₅alkylene;
R₃₈ is hydrogen, C₁-C₄alkyl, C₁-C₄haloalkyl or C₃-C₆cycloalkyl;
R₃₉ is hydrogen, C₁-C₈alkyl, C₃-C₈alkenyl, C₃-C₈alkynyl, C₁-C₄haloalkyl or C₃-C₈haloalkenyl;
R₄₀ is hydrogen, C₁-C₄alkyl, C₁-C₄haloalkyl, C₁-C₈alkylthio, —C(O)—C(O)OC₁-C₄alkyl or C₃-C₆cycloalkyl;
R₄₁ is hydrogen, C₁-C₈alkyl, C₃-C₈alkenyl, C₃-C₈alkynyl, C₁-C₆alkoxy-C₁-C₆alkyl, C₁-C₈alkylcarbonyl, C₁-C₈alkoxycarbonyl, C₃-C₈alkenyloxycarbonyl, C₁-C₆alkoxy-C₁-C₆alkoxycarbonyl, C₁-C₆alkylthio-C₁-C₆alkyl, C₁-C₆alkylsulfinyl-C₁-C₆alkyl or C₁-C₆alkylsulfonyl-C₁-C₆alkyl; or
R₄₁ is phenyl or phenyl-C₁-C₆alkyl, it being possible for the phenyl ring in turn to be mono-, di- or tri-substituted by halogen, C₁-C₄alkyl, C₁-C₄haloalkyl, C₁-C₄alkoxy, —CN, —NO₂ or by —S(O)₂C₁-C₈alkyl, or
R₄₁ is C₁-C₈alkyl mono-, di- or tri-substituted by —COOH, C₁-C₈alkoxycarbonyl, C₁-C₆alkylamino, di(C₁-C₆alkyl)amino or by —CN;
R₄₂ is hydrogen, C₁-C₈alkyl, C₃-C₈alkenyl or C₃-C₈alkynyl, or is C₁-C₈alkyl mono-, di- or tri-substituted by halogen, —CN or by C₁-C₄alkoxy;
R₄₃ and R₄₄ are each independently of the other C₁-C₈alkyl, C₃-C₈alkenyl or C₃-C₈alkynyl, or C₁-C₈alkyl mono-, di- or tri-substituted by halogen, —CN or by C₁-C₄alkoxy;
R₄₅ is C₁-C₈alkyl, C₁-C₈alkyl mono-, di- or tri-substituted by halogen, —ON or by C₁-C₄alkoxy, or is C₃-C₈alkenyl or C₃-C₈alkynyl, or
R₄₅ is phenyl, it being possible for the phenyl ring to be mono-, di- or tri-substituted by halogen, C₁-C₄alkyl, C₁-C₄haloalkyl, C₁-C₄alkoxy, —CN, NO₂, C₁-C₈alkylthio, C₁-C₈alkylsulfinyl or by C₁-C₈alkylsulfonyl;
R₄₆ is hydrogen, C₁-C₈alkyl, C₃-C₈alkenyl, C₃-C₈alkynyl or C₁-C₄haloalkyl;
R₄₇ is hydrogen, C₁-C₈alkyl, C₁-C₄alkoxy, C₃-C₈alkenyl or C₃-C₈alkynyl, or is C₁-C₈alkyl mono-, di- or tri-substituted by halogen, —CN, C₁-C₄alkoxy, C₁-C₈alkoxycarbonyl, —NH₂, C₁-C₄alkylamino, di(C₁-C₄alkyl)amino, —NR₄₈COR₄₉, —NR₅₀SO₂R₅₁ or by —NR₅₂CO₂R₅₃, or
R₄₇ is phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl;
p is 0 or 1;
R₄₈, R₄₉, R₅₀, R₅₁, R₅₂ and R₅₃ are each independently of the others hydrogen, C₁-C₈alkyl, phenyl, benzyl or naphthyl, it being possible for the three last-mentioned aromatic radicals in turn to be mono-, di- or tri-substituted by halogen, C₁-C₈alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₅₄ and R₅₅ are each independently of the other hydrogen, C₁-C₈alkyl, or phenyl which may in turn be mono-, di- or tri-substituted by halogen, C₁-C₄alkyl, C₁-C₄haloalkyl, C₁-C₄alkoxy, —CN, —NO₂, C₁-C₈alkylthio, C₁-C₈alkylsulfinyl or by C₁-C₈alkylsulfonyl;
R₅₆ is hydrogen, C₁-C₈alkyl, C₁-C₄haloalkyl, C₁-C₄alkoxy, C₃-C₈alkenyl, C₃-C₈alkynyl or benzyl, it being possible for benzyl in turn to be mono-, di- or tri-substituted by halogen, C₁-C₄alkyl, C₁-C₄haloalkyl, C₁-C₄alkoxy, —CN, —NO₂, C₁-C₈alkylthio, C₁-C₈alkylsulfinyl or by C₁-C₈alkylsulfonyl;
R₅₇ is C₁-C₈alkyl, C₁-C₄haloalkyl, phenyl, benzyl or naphthyl, it being possible for the last three aromatic rings to be mono-, di- or tri-substituted by halogen, C₁-C₄alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₅₈ and R₅₉ are each independently of the other C₁-C₈alkyl, C₃-C₈alkynyl, C₃-C₈alkynyl, phenyl, benzyl or naphthyl, it being possible for the last three aromatic rings to be mono-, di- or tri-substituted by halogen, C₁-C₄alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₆₀ and R₆₁ are each independently of the other hydrogen or C₁-C₆alkyl;
R₆₂, R₆₃ and R₆₄ are each independently of the others hydrogen or C₁-C₈alkyl, or
R₆₃ and R₆₄ together form a C₂-C₅alkylene bridge;
R₆₅, R₆₆, R₆₇, R₆₈, R₆₉ and R₇₀ are each independently of the others hydrogen or C₁-C₈alkyl, or
R₆₅ and R₆₆, or R₆₇ and R₆₈, or R₆₉ and R₇₀ in each case together form a C₂-C₅alkylene bridge; or
each R₂ independently is C₁-C₈alkyl, or is C₁-C₈alkyl mono-, di- or tri-substituted by halogen, —CN, —N₃, —SCN, —NO₂, —NR₇₁R₇₂, —CO₂R₇₃, —CONR₇₄R₇₅, —COR₇₆, —C(R₇₇)═NOR₇₈, —C(S)NR₇₉R₈₀, —C(C₁-C₄alkylthio)═NR₈₁, —OR₈₂—SR₈₃, —SOR₈₄, —SO₂R₈₅, —O(SO₂)R₈₆, —N(R₈₇)CO₂R₈₈, —N(R₈₉)COR₉₀, —S⁺(R₉₁)₂, —N⁺(R₉₂)₃, —Si(R₉₃)₃ or by C₃-C₆cycloalkyl; or
each R₂ independently is C₁-C₈alkyl substituted by a 5- to 7-membered heterocyclic ring system which may be aromatic or partially or fully saturated and may contain from 1 to 4 hetero atoms selected from nitrogen, oxygen and sulfur, it being possible for that hetero-cyclic ring system in turn to be mono-, di- or tri-substituted by halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or by C₁-C₆alkylsulfonyl; or
each R₂ independently is C₂-C₈alkenyl, or is C₂-C₈alkenyl mono-, di- or tri-substituted by —CN, —NO₂, —CO₂R₉₄, —CONR₉₅R₉₆, —COR₉₇, —C(R₉₈)═NOR₉₉, —C(S)NR₁₀₀R₁₀₁, —C(C₁-C₄alkylthio)═NR₁₀₂, —OR₁₀₃, —Si(R₁₀₄)₃ or by C₃-C₆cycloalkyl; or
each R₂ independently is C₂-C₈alkynyl, or is C₂-C₈alkynyl mono-, di- or tri-substituted by halogen, —CN, —CO₂R₁₀₅, —CONR₁₀₆R₁₀₇, —COR₁₀₈, —C(R₁₀₉)═NOR₁₁₀, —C(S)NR₁₁₁R₁₁₂, —C(C₁-C₄alkylthio)═NR₁₁₃, —OR₁₁₄, —Si(R₁₁₅)₃ or by C₃-C₆cycloalkyl; or
each R₂ independently is C₃-C₆cycloalkyl, or is C₃-C₆cycloalkyl mono-, di- or tri-substituted by halogen, —CN, —CO₂R₁₁₆, —CONR₁₁₇R₁₁₈, —COR₁₁₉, —C(R₁₂₀)═NOR₁₂₁, —C(S)NR₁₂₂R₁₂₃ or by —C(C₁-C₄alkylthio)═NR₁₂₄; or
two adjacent R₂ substituents together form a C₁-C₇alkylene bridge which may be interrupted by from 1 to 3 hetero atoms selected from oxygen, nitrogen and sulfur and may be mono-, di- or tri-substituted by halogen, C₁-C₆alkyl or by C₁-C₆alkoxy, the total number of ring atoms being at least 5 and at most 9; or
two adjacent R₂ substituents together form a C₂-C₇alkenylene bridge which may be interrupted by from 1 to 3 hetero atoms selected from oxygen, nitrogen and sulfur and may be mono-, di- or tri-substituted by halogen, C₁-C₆alkyl or by C₁-C₆alkoxy, the total number of ring atoms being at least 5 and at most 9;
R₇₁ is hydrogen or C₁-C₈alkyl;
R₇₂ is hydrogen, C₁-C₈alkyl, C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₇₁ and R₇₂ together are a C₂-C₅alkylene chain which may be interrupted by an oxygen or sulfur atom;
R₇₃ is hydrogen, C₁-C₈alkyl, C₃-C₈alkenyl or C₃-C₈alkynyl, or is C₁-C₈alkyl, C₃-C₈alkenyl or C₃-C₈alkynyl mono-, di- or tri-substituted by halogen, C₁-C₄alkoxy or by phenyl, it being possible for phenyl in turn to be mono-, di- or tri-substituted by halogen, C₁-Q₄alkyl, C₁-C₄-haloalkyl, C₁-C₄alkoxy, —CN, —NO₂, C₁-C₄alkylthio, C₁-C₄alkylsulfinyl or by C₁-C₄alkylsulfonyl;
R₇₄ is hydrogen or C₁-C₈alkyl;
R₇₅ is hydrogen, C₁-C₈alkyl or C₃-C₇cycloalkyl, or is C₁-C₈alkyl mono-, di- or tri-substituted by —COOH, C₁-C₈alkoxycarbonyl, C₁-C₆alkoxy or by —CN; or
R₇₅ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₇₄ and R₇₅ together are a C₂-C₅alkylene chain which may be interrupted by an oxygen or sulfur atom;
R₇₆ is hydrogen, C₁-C₄alkyl, C₁-C₄haloalkyl or C₃-C₆cycloalkyl;
R₇₇ is hydrogen, C₁-C₄alkyl, C₁-C₄haloalkyl or C₃-C₆cycloalkyl;
R₇₈ is hydrogen, C₁-C₈alkyl, C₃-C₈alkenyl, C₃-C₈alkynyl, C₁-C₄haloalkyl or C₃-C₆haloalkenyl; and
R₇₉ is hydrogen or C₁-C₈alkyl;
R₈₀ is hydrogen or C₁-C₈alkyl, or is C₁-C₈alkyl mono-, di- or tri-substituted by —COOH, C₁-C₈alkoxycarbonyl or by —CN; or
R₈₀ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₇₉ and R₈₀ together are C₂-C₅alkylene;
R₈₁ is hydrogen or C₁-C₈alkyl;
R₈₂ is —Si(C₁-C₆alkyl)₃, C₃-C₈alkenyl or C₃-C₈alkynyl, or is C₁-C₈alkyl which is mono-, di- or tri-substituted by halogen, —CN, —NH₂, C₁-C₆alkylamino, di(C₁-C₆alkyl)amino or by C₁-C₄alkoxy;
R₈₃ is hydrogen, C₁-C₈alkyl, C₃-C₈alkenyl or C₃-C₈alkynyl, or is C₁-C₈alkyl which is mono-, di- or tri-substituted by halogen, —CN, —NH₂, C₁-C₆alkylamino, di(C₁-C₆alkyl)amino or by C₁-C₄alkoxy;
R₈₄, R₈₅ and R₈₆ are each independently of the others C₁-C₈alkyl, C₃-C₈alkenyl or C₃-C₈-alkynyl, or C₁-C₈alkyl which is mono-, di- or tri-substituted by halogen, —CN or by C₁-C₄-alkoxy;
R₈₇ and R₈₉ are each independently of the other hydrogen, C₁-C₈alkyl or C₁-C₈alkoxy;
R₈₈ is C₁-C₈alkyl;
R₉₀ is hydrogen or C₁-C₈alkyl;
R₉₁ is C₁-C₄alkyl;
R₉₂ and R₉₃ are each independently of the other C₁-C₆alkyl;
R₉₄ is hydrogen or is C₁-C₈alkyl, C₃-C₈alkenyl or C₃-C₈alkynyl, each of which may be mono-, di- or tri-substituted by halogen, C₁-C₄alkoxy or by phenyl, it being possible for phenyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl;
R₉₅ is hydrogen or C₁-C₈alkyl;
R₉₆ is hydrogen or C₁-C₈alkyl, or is C₁-C₈alkyl mono-, di- or tri-substituted by —COOH, C₁-C₈alkoxycarbonyl or by —CN; or
R₉₆ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₉₅ and R₉₆ together are C₂-C₅alkylene;
R₉₇ and R₉₈ are each independently of the other hydrogen, C₁-C₄alkyl, C₁-C₄haloalkyl or C₃-C₆cycloalkyl;
R₉₉ is hydrogen, C₁-C₈alkyl, C₃-C₈alkenyl, C₃-C₈alkynyl, C₁-C₄haloalkyl or C₃-C₆haloalkenyl;
R₁₀₀ is hydrogen or C₁-C₈alkyl;
R₁₀₁ is hydrogen or C₁-C₈alkyl, or is C₁-C₈alkyl mono-, di- or tri-substituted by —COOH, C₁-C₈alkoxycarbonyl or by —CN; or
R₁₀₁ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₀₀ and R₁₀₁ together are C₂-C₅alkylene;
R₁₀₂ is hydrogen or C₁-C₈alkyl;
R₁₀₃ is hydrogen, C₁-C₈alkyl, —Si(C₁-C₆alkyl)₃, C₃-C₈alkenyl or C₃-C₈alkynyl;
R₁₀₄ is C₁-C₆alkyl;
R₁₀₅ is hydrogen or is C₁-C₈alkyl, C₃-C₈alkenyl or C₃-C₈alkynyl, each of which may be mono-, di- or tri-substituted by halogen, C₁-C₄alkoxy or by phenyl, it being possible for phenyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl;
R₁₀₆ is hydrogen or C₁-C₈alkyl;
R₁₀₇ is hydrogen or C₁-C₈alkyl, or is C₁-C₈alkyl mono-, di- or tri-substituted by —COOH, C₁-C₈alkoxycarbonyl or by —CN, or
R₁₀₇ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₀₆ and R₁₀₇ together are C₂-C₅alkylene;
R₁₀₈ is hydrogen, C₁-C₄alkyl, C₁-C₄haloalkyl or C₃-C₆cycloalkyl;
R₁₀₉ is hydrogen, C₁-C₄alkyl, C₁-C₄haloalkyl or C₃-C₆cycloalkyl,
R₁₁₀ is hydrogen, C₁-C₈alkyl, C₃-C₈alkenyl, C₃-C₈alkynyl, C₁-C₄haloalkyl or C₃-C₆haloalkenyl;
R₁₁₁ is hydrogen or C₁-C₈alkyl;
R₁₁₂ is hydrogen or C₁-C₈alkyl, or is C₁-C₈alkyl mono-, di- or tri-substituted by —COOH, C₁-C₈alkoxycarbonyl or by —CN; or
R₁₁₂ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₁₁ and R₁₁₂ together are C₂-C₅alkylene;
R₁₁₃ is hydrogen or C₁-C₈alkyl;
R₁₁₄ is hydrogen, C₁-C₈alkyl, —Si(C₁-C₆alkyl)₃, C₃-C₈alkenyl or C₃-C₈alkynyl;
R₁₁₅ is C₁-C₆alkyl;
R₁₁₆ is hydrogen or is C₁-C₈alkyl, C₃-C₈alkenyl or C₃-C₈alkynyl, each of which may be mono-, di- or tri-substituted by halogen, C₁-C₄alkoxy or by phenyl, it being possible for phenyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl;
R₁₁₇ is hydrogen or C₁-C₈alkyl;
R₁₁₈ is hydrogen or C₁-C₈alkyl, or is C₁-C₈alkyl mono-, di- or tri-substituted by —COOH, C₁-C₈alkoxycarbonyl or by —CN; or
R₁₁₈ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₁₇ and R₁₁₈ together are C₂-C₅alkylene;
R₁₁₉ is hydrogen, C₁-C₄alkyl, C₁-C₄haloalkyl or C₃-C₆cycloalkyl;
R₁₂₀ is hydrogen, C₁-C₄alkyl, C₁-C₄haloalkyl or C₃-C₆cycloalkyl;
R₁₂₁ is hydrogen, C₁-C₈alkyl, C₃-C₈alkenyl, C₃-C₈alkynyl, C₁-C₄haloalkyl or C₃-C₆haloalkenyl;
R₁₂₂ is hydrogen or C₁-C₈alkyl;
R₁₂₃ is hydrogen or C₁-C₈alkyl, or is C₁-C₈alkyl mono-, di- or tri-substituted by —COOH, C₁-C₈alkoxycarbonyl or by —CN; or
R₁₂₃ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₂₂ and R₁₂₃ together are C₂-C₅alkylene; and
R₁₂₄ is hydrogen or C₁-C₈alkyl.

Preference is given also to compounds of formula I wherein each R₁ independently is halogen, —CN, —NO₂, —C(R₁₀)═NOR₁₁, —OR₁₃, —SO₂R₁₆, —OSO₂R₁₇, C₁-C₈alkyl or C₂-C₈alkenyl, or is C₁-C₈alkyl mono-, di- or tri-substituted by halogen or by —CN; R₁₀ is hydrogen or C₁-C₄alkyl; and R₁₁ is C₁-C₈alkyl.

Preference is given likewise to compounds of formula I wherein Q is a group Q₁, Q₂, Q₃ or Q₅. Among those compounds special preference is given to those wherein Q is a group Q, or Q₂.

Preference is given furthermore to those compounds of formula I wherein each R₂ independently is halogen, —CN, —SCN, —OCN, —N₃, —CONR₃₆R₃₇, —C(R₃₈)═NOR₃₉, —COR₄₀, —OR₄₁, —OSO₂R₄₅, —N([CO]ₚR₄₆)COR₄₇, —N(R₅₆)SO₂R₅₇, —N(SO₂R₅₈)SO₂R₅₉, —N═C(OR₆₀)R₆₁ or C₁-C₈alkyl, or is C₁-C₈alkyl mono-, di- or tri-substituted by halogen, —CN, —N₃, —SCN, —CONR₇₄R₇₅, —COR₇₆, —C(R₇₇)═NOR₇₈, —C(S)NR₇₉R₈₀, —OR₈₂, —SOR₈₄, —SO₂R₈₅ or by —N(R₈₉)COR₉₀.

The compounds of formula Ia 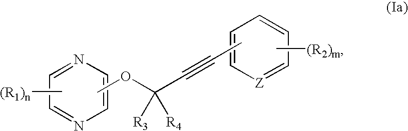
wherein R₁, R₂, R₃, R₄, Z, m and n are as defined for formula I, can be prepared analogously to known methods described, for example, in “Palladium in Heterocyclic Chemistry” from Tetrahedron Organic Chemistry Series 52, A Guide for the Synthetic Chemist, Editors Jie Jack Li and Gordon W. Gribble, Pergamon 2000, Tetrahedron Lett. 1986 (27), 1171; Tetrahedron Organic Chemistry 2000 (20), 359-362; ibid. 2000 (20), 390-394; and K. Sonogashira in Comprehensive Organic, Synthesis, Editors I. Fleming et al., Oxford 1991, Vol. 3, page 521 ff., for example by reacting a compound of formula IIa 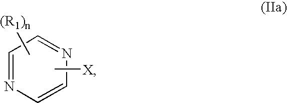
wherein R₁ and n are as defined for formula I (Q=Q₁) and X is halogen, with a compound of formula IIIa 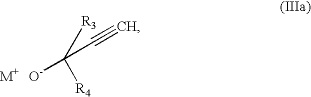
wherein R₃ and R₄ are as defined for formula I and M⁺ is an alkali metal cation such as, for example, a lithium, sodium or potassium cation, to form a compound of formula IVa 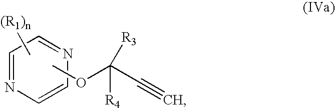
wherein R₁, R₃, R₄ and n are as defined for formula I, and then coupling that compound with a compound of formula V 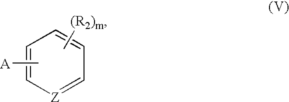
wherein Z, R₂ and m are as defined for formula I and A is a leaving group such as, for example, halogen or trifluoromethanesulfonate, in the presence of a palladium catalyst.

Preparation of the compounds of formula I wherein Q is a group Q₂, that is to say compounds of formula Ib 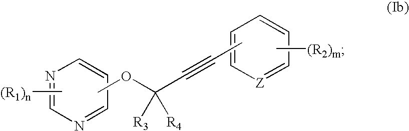
wherein Q is a group Q₃, that is to say compounds of formula Ic 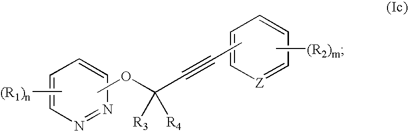
wherein Q is a group Q₄, that is to say compounds of formula Id 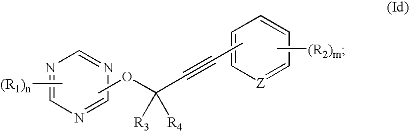
wherein Q is a group Q₅, that is to say compounds of formula Ie 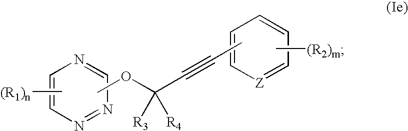
and wherein Q is a group Q₆, that is to say compounds of formula If 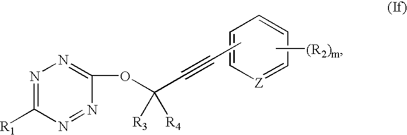
is carried out in a manner analogous to that described above by way of example for the compounds of formula Ia (Q=Q₁).

These preparation procedures for the compounds of formula I are illustrated in Reaction Schemes 1, 2 and 3 specifically using the example of the compound of formula Ia (Q=Q₁). It is generally true of all three Reaction Schemes that the various substituents R₁ and R₂ in the compounds of formulae IIa and V either are already present at the outset or, however, they may be successively introduced only later in the reaction sequence, for example by means of nucleophilic or electrophilic aromatic substitution.

The same is also true of the analogous preparation of compounds of formulae Ib, Ic, Id, le and If (Q is a group Q₂ to Q₆), starting from the respective compounds of formulae IIb, IIc, IId, IIe and IIf:

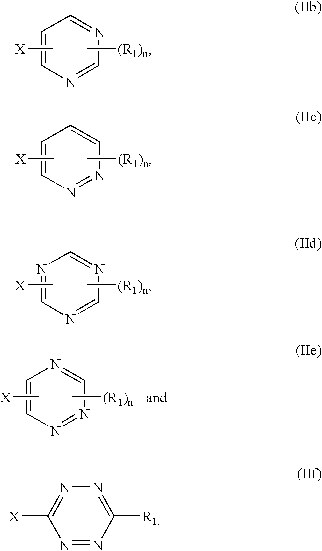

In accordance with Reaction Scheme 1, the compounds of formula Ia can be obtained, for example, by reaction of substituted propargyl ethers of formula IVa with compounds of formula V by means of Sonogashira coupling.

The propargyl ethers of formula IVa can, for their part, be obtained by a nucleophilic aromatic substitution reaction of compounds of formula IIa wherein X is halogen with alcoholates of formula IIIa. Such substitution reactions are standard methods and may be carried out, for example, in analogy to Tetrahedron 1972 (28), 4155; Heterocycles 1990 (31), 1275 (for Q=Q₁); J. Org. Chem. 1961 (26), 2764 (for Q=Q₂); Tetrahedron Lett. 1996 (37), 4065; Heterocyclic Chem. 1995 (32), 1057 (for Q=Q₃); J. Am. Chem. Soc. 1951 (73), 2986 (for Q=Q₄); Collect Czech. Chem. 1975 (40), 2680 (for Q=Q₅); and Tetrahedron Lett. 1985 (26), 4355 (for Q=Q₆).

In the next step, the propargyl ethers of formula IVa are coupled with substituted phenyl or pyridine derivatives of formula V (Z is ═N— or ═C(R₂)—) under typical Sonogashira conditions (K. Sonogashira in Comprehensive Organic Synthesis 1991, Vol. 3, page 521 ff.; J. Org. Chem. 1998 (63), 8551-8553). Suitable catalyst mixtures are, for example, tetrakis(triphenylphosphine)palladium or bis(triphenylphosphine)palladium(II) dichloride together with copper(I) iodide (CuI); suitable bases are preferably amines, for example triethylamine, diethylamine or diisopropylethylamine.

The phenyl or pyridine derivatives of formula V preferably have a leaving group A, A being, for example, halogen or trifluoromethanesulfonate (Tetrahedron Organic Chemistry 2000 (20), 209-213; J. Org. Chem. 1998 (63), 8551-8553) and Tetrahedron Lett. 1986 (27), 1171. Solvents used for the Sonogashira reaction are usually ethers, for example tetrahydrofuran, chlorinated hydrocarbons, for example chloroform, or dipolar aprotic solvents, for example dimethylformamide or dimethyl sulfoxide, and also amines, for example triethylamine or piperidine.

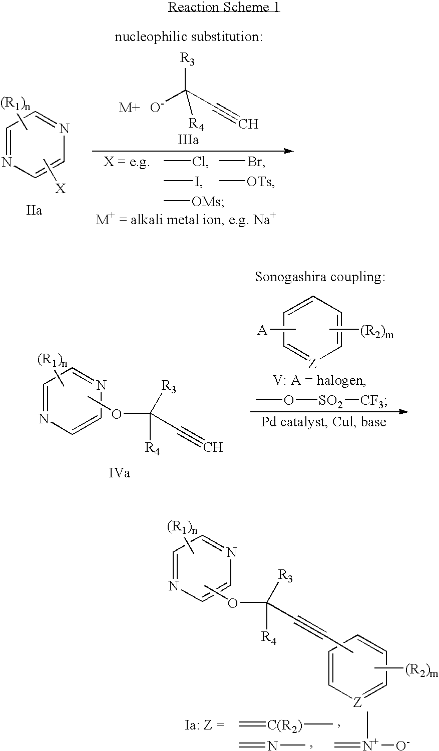

Pd-catalysed cross-coupling of appropriately substituted phenyl or pyridine derivatives of formula V with propargyl alcohols of formula III in accordance with K. Sonogashira, as shown in diagrammatic form in Reaction Scheme 2, results in compounds of formula VII. Such reactions are documented, for example, in Tetrahedron Organic Chemistry 2000 (20), 209-213 for pyridine derivatives and in J. Org. Chem. 1988 (53), 386; ibid. 1998 (63), 8551-8553; and Tetrahedron Lett. 1986 (27), 1171 for phenyl derivatives. Subsequent reaction of the propargyl alcohols of formula VII with pyrazine derivatives of formula IIa wherein X is halogen yields the compounds of formula Ia (Q=Q₁).

The other compounds of formula I wherein Q is a group Q₂, Q₃, Q₄, Q₅ or Q₆ can also be prepared in a manner analogous to that shown in Reaction Scheme 2.

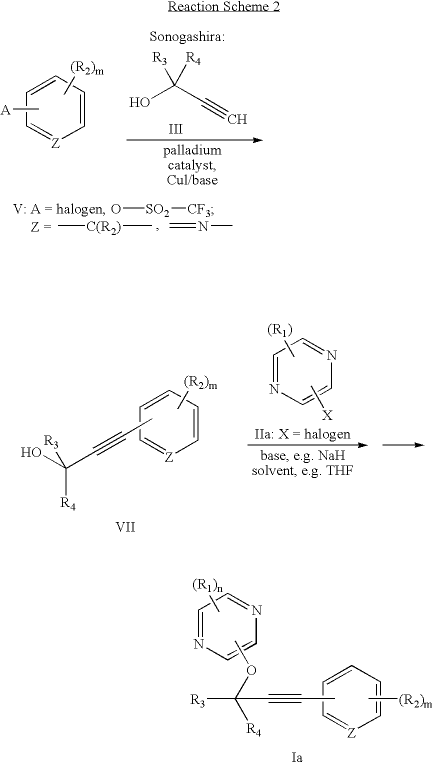

The compounds of formula I can also be obtained by further methods such as, for example, that shown in Reaction Scheme 3.

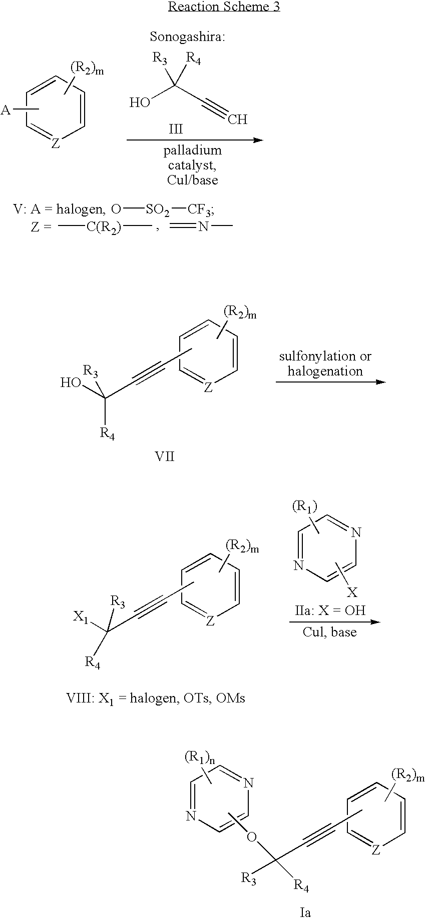

Activation of the alcohol of formula VII according to Reaction Scheme 3 is carried out, for example, by sulfonylation or halogenation.

Sulfonylation of the alcohol of formula VII is a standard reaction and can be carried out, for example, using a sulfonic acid chloride, for example mesyl chloride or para-toluenesulfonic acid chloride (p-TosCl), in the presence of a tertiary amine, e.g. triethylamine, or an aromatic amine, e.g. pyridine, in a solvent such as, for example, a chlorinated hydrocarbon, e.g. carbon tetrachloride or methylene chloride, or an amine, e.g. pyridine. Such reactions are generally known and are described, for example, in J. Org. Chem. 1997 (62), 8987; J. Het. Chem. 1995 (32), 875-882; and Tetrahedron Lett. 1997 (38), 8671-8674. Halogenation of the alcohol of formula VII can be carried out in analogy to standard methods. For example, bromination can be successfully carried out using carbon tetrabromide in the presence of triphenylphosphine (Synthesis 1998, 1015-1018) in methylene chloride. Chlorination can be successfully carried out using mineral acids, for example using concentrated hydrochloric acid (J. Org. Chem. 1955 (20), 95), or using para-toluenesulfonic acid chloride in the presence of an amine, for example triethylamine, in a solvent, for example methylene chloride (Tetrahedron Lett. 1984 (25), 2295). Preparation of the compound of formula Ia according to Reaction Scheme 3 can be carried out analogously to Synthesis 1995, 707-712 and Tetrahedron Lett. 1994 (35), 6405-6408, for example by means of copper iodide-catalysed etherification of the hydroxy-pyrazine of formula IIa in the presence of the tosylate (X₁=OTs) or mesylate (X₁=OMs) or halide (X₁=halogen) of formula VIII. Suitable solvents are dimethylformamide or acetonitrile; suitable bases are preferably potassium carbonate or 1,8-diazabicylo[5.4.0]undec-7-ene (DBU). The etherification can also be carried out in halogenated or aromatic hydrocarbons as solvent, for example in chloroform or in benzene, in the presence of silver carbonate as base. Such selective O-alkylation reactions in the presence of a ring nitrogen atom are described, for example, in Synth. Commun. 1994 (24), 1367 and Heterocycles 1990 (31), 819.

A further method by which the compounds of formula I can be prepared is performed with the aid of Mitsunobu reaction in a manner analogous to that described, for example, in Synthesis 1981 (1); Tetrahedron Lett. 35, 2819-2822 (1994); and Chem. Letters 1994, 539 (with TMAD reagent as a replacement for the DEAD in the two aforesaid references). This synthesis route is illustrated in Reaction Scheme 4 using the example of the compounds of formula Ia (pyrazinyloxy-alkyne derivatives).

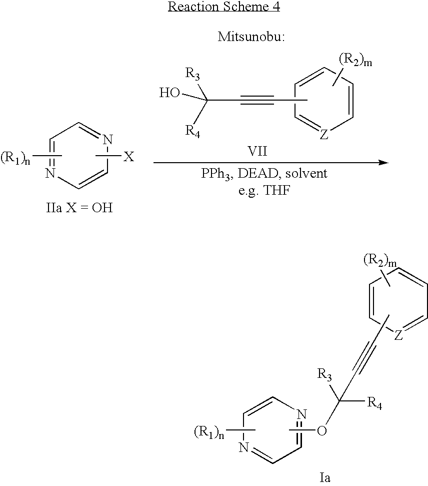

The following applies to the individual reaction steps according to Reaction Schemes 1 to 4: The reactions resulting in the compounds of formula I are advantageously performed in aprotic, inert, organic solvents. Such solvents are hydrocarbons, such as benzene, toluene, xylene or cyclohexane, chlorinated hydrocarbons, such as dichloromethane, trichloromethane, tetrachloromethane or chlorobenzene, ethers, such as diethyl ether, ethylene glycol dimethyl ether, diethylene glycol dimethyl ether, tetrahydrofuran or dioxane, nitriles, such as acetonitrile or propionitrile, amides, such as N,N-dimethylformamide, diethyl-formamide or N-methylpyrrolidinone. The reaction temperatures are preferably from −20° C. to +120° C. The reactions are generally slightly exothermic and can usually be carried out at room temperature. For reducing the reaction time or also for initiation of the reaction, heating, up to the boiling point of the reaction mixture, may, where appropriate, be carried out for a short time. It is also possible for the reaction times to be reduced by adding a few drops of base as reaction catalyst. Suitable bases are especially tertiary amines, such as trimethylamine, triethylamine, quinuclidine, 1,4-diazabicyclo[2.2.2]octane, 1,5-diazabicyclo[4.3.0]non-5-ene or 1,5-diazabicyclo[5.4.0]undec-7-ene. However, there may also be used as bases inorganic bases, such as hydrides, e.g. sodium hydride or calcium hydride, hydroxides, e.g. sodium hydroxide or potassium hydroxide, carbonates, e.g. sodium carbonate or potassium carbonate, or hydrogen carbonates, e.g. potassium hydrogen carbonate or sodium hydrogen carbonate.

The compounds of formula I may, in conventional manner, be isolated by concentrating and/or evaporating off the solvent and purified by recrystallising or triturating the solid residue in solvents in which they are not readily soluble, such as ethers, aromatic hydrocarbons or chlorinated hydrocarbons.

The starting compounds of formula IIa used in Reaction Schemes 1, 2 and 3, and the corresponding starting compounds of formulae IIb, IIc, IId, IIe and IIf for preparation of the compounds of formulae Ib, Ic, Id, Ie and If are for the most part known or they can be prepared in analogy to known methods as described, for example, in J. Org. Chem. 1997 (62), 9112; ibid. 1958 (23), 1522; J. Chem. Soc. 1948, 2191; Bull. Soc. Chim. Fr. 1957, 1009; J. Am. Chem. Soc. 74, 1580-1582 (1952); U.S. Pat. No. 5,547,919; J. Chem. Soc. 1960, 4590; J. Org. Chem. 1963 (28), 1682; J. Heterocycl. Chem. 1994 (31), 1177; and ibid 1982 (19), 1061.

The starting compounds of formulae III and IIIa are likewise known and in some cases are commercially available, or they can be prepared in analogy to known methods.

The compounds of formula V are likewise known and in some cases are commercially available. Examples of substituted compounds of formula V wherein Z is ═N— are described, for example, in Tetrahedron Organic Chemistry 20, 209 (2000).

The compounds of formulae IV (and IVa to IVf) and VII are novel. The present invention accordingly relates also to those compounds.

For the use according to the invention of the compounds of formula I, or of compositions comprising them, there come into consideration all methods of application customary in agriculture, for example pre-emergence application, post-emergence application and seed dressing, and also various methods and techniques such as, for example, the controlled release of active ingredient. For that purpose a solution of the active ingredient is applied to mineral granule carriers or polymerised granules (urea/formaldehyde) and dried. If required, it is also possible to apply a coating (coated granules), which allows the active ingredient to be released in metered amounts over a specific period of time.

The compounds of formula I may be used as herbicides in their unmodified form, that is to say as obtained in the synthesis, but they are preferably formulated in customary manner together with the adjuvants conventionally employed in formulation technology, for example into emulsifiable concentrates, directly sprayable or dilutable solutions, dilute emulsions, wettable powders, soluble powders, dusts, granules or microcapsules. Such formulations are described, for example, on pages 9 to 13 of WO 97/34485. As with the nature of the compositions, the methods of application, such as spraying, atomising, dusting, wetting, scattering or pouring, are chosen in accordance with the intended objectives and the prevailing circumstances.

The formulations, that is to say the compositions, preparations or mixtures comprising the compound (active ingredient) of formula I, or at least one compound of formula I, and, usually, one or more solid or liquid formulation adjuvants, are prepared in known manner, e.g. by homogeneously mixing and/or grinding the active ingredient(s) with the formulation adjuvants, for example solvents or solid carriers. Surface-active compounds (surfactants) may also be used in addition in the preparation of the formulations. Examples of solvents and solid carriers are given, for example, on page 6 of WO 97/34485.

Depending upon the nature of the compound of formula I to be formulated, suitable surface-active compounds are non-ionic, cationic and/or anionic surfactants and surfactant mixtures having good emulsifying, dispersing and wetting properties. Examples of suitable anionic, non-ionic and cationic surfactants are listed, for example, on pages 7 and 8 of WO 97/34485. In addition, the surfactants conventionally employed in formulation technology, which are described, inter alia, in “McCutcheon's Detergents and Emulsifiers Annual” MC Publishing Corp., Ridgewood N.J., 1981, Stache, H., “Tensid-Taschenbuch”, Carl Hanser Verlag, Munich/Vienna, 1981, and M. and J. Ash, “Encyclopedia of Surfactants”, Vol. 1-11, Chemical Publishing Co., New York, 1980-81, are also suitable for the preparation of the herbicidal compositions according to the invention.

The herbicidal formulations generally contain from 0.1 to 99% by weight, especially from 0.1 to 95% by weight, of herbicide, from 1 to 99.9% by weight, especially from 5 to 99.8% by weight, of a solid or liquid formulation adjuvant, and from 0 to 25% by weight, especially from 0.1 to 25% by weight, of a surfactant. Whereas commercial products will preferably be formulated as concentrates, the end user will normally employ dilute formulations. The compositions may also comprise further ingredients, such as stabilisers, for example vegetable oils or epoxidised vegetable oils (epoxidised coconut oil, rapeseed oil or soybean oil), anti-foams, for example silicone oil, preservatives, viscosity regulators, binders, tackifiers, and also fertilisers or other active ingredients.

The compounds of formula I are generally applied to the plant or the locus thereof at rates of application of from 0.001 to 4 kg/ha, especially from 0.005 to 2 kg/ha. The concentration required to achieve the desired effect can be determined by experiment. It is dependent on the nature of the action, the stage of development of the cultivated plant and of the weed and on the application (place, time, method) and may vary within wide limits as a function of those parameters.

The compounds of formula I are distinguished by herbicidal and growth-inhibiting properties, allowing them to be used in crops of useful plants, especially cereals, cotton, soybeans, sugar beet, sugar cane, plantation crops, rape, maize and rice, and also for non-selective weed control. The term “crops” is to be understood as including also crops that have been made tolerant to herbicides or classes of herbicides as a result of conventional methods of breeding or genetic engineering techniques. The weeds to be controlled may be either monocotyledonous or dicotyledonous weeds, such as, for example, Stellaria, Nasturtium, Agrostis, Digitaria, Avena, Setaria, Panicum, Sinapis, Lolium, Solanum, Echinochloa, Scirpus, Euphorbia, Monochoria, Sagittaria, Bromus, Alopecurus, Sorghum halepense, Rottboellia, Cyperus, Abutilon, Sida, Xanthium, Amaranthus, Chenopodium, Ipomoea, Chrysanthemum, Galium, Viola and Veronica.

The following Examples further illustrate but do not limit the invention.

### PREPARATION EXAMPLES

### Example P1

### Preparation of 2-mercapto-5-methoxy-3H-pyrimidin-4-one (see J. Chem. Soc. 1960, 4590)

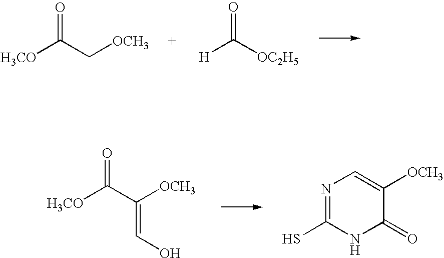

A mixture of 9.9 ml (0.1 mol) of methoxyacetic acid methyl ester and 8.1 ml (0.1 mol) of ethyl formate is added dropwise to a stirred suspension of 2.3 g (0.1 mol) of sodium in 30 ml of toluene, the temperature being kept below 30° C. On the following day, the toluene phase is decanted off and 15 ml of ethanol and 7.6 g (0.1 mol) of thiourea are added to the crude, viscous sodium salt of 3-hydroxy-2-methoxyacrylic acid methyl ester. The resulting mixture is stirred at 20° C. for 1 hour and is then heated at reflux temperature for 5 hours. After cooling, the solid formed is dissolved in 50 ml of water and the resulting solution is rendered neutral with 6N hydrochloric acid. The desired title compound precipitates out and can, after drying at 100° C., be obtained in a yield of 8.3 g (52% of theory). The product can be further used directly for further reactions.

For purification, the crude product is recrystallised from water, the desired title compound being obtained in the form of needles having a melting point of 280-281° C. (decomposition).

¹H NMR (300 MHz, DMSO-d₆): 12.526 ppm (broad singlet, 1H); 12.136 ppm (broad singlet, 1H); 7.016 ppm (s, 1H); 3.630 ppm (s, 3H).

### Example P2

### Preparation of 5-methoxy-3H-pyrimidin-4-one (see J. Chem. Soc. 1960, 4590)

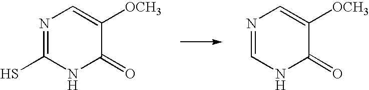

3.9 g of Raney nickel (slurry) are added to a hot solution of 4.1 g (0.026 mol) of 2-mercapto-5-methoxy-3H-pyrimidin-4-one (Example P1) in 60 ml of water. After vigorous stirring for 8 hours at reflux temperature, the reaction mixture is filtered and the combined filtrates and washing fractions are concentrated by evaporation on a hot water bath. The residue obtained is recrystallised from ethanol in the presence of activated carbon. The desired target compound is obtained in a yield of 1.9 g (69% of theory) in the form of needles having a melting point of 206-208° C.

¹H NMR (300 MHz, DMSO-d₆): 7.828 ppm (s, 1H); 7.527 ppm (s, 1H); 3.728 ppm (s, 3H).

### Example P3

### Preparation of 4-chloro-5-methoxy-pyrimidine (see J. Chem. Soc. 1960, 4590 and U.S. Pat. No. 5,547,919)

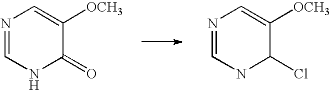

A suspension of 1.9 g (0.015 mol) of 5-methoxy-3H-pyrimidin-4-one (Example P2) in 4.2 ml (0.046 mol) of phosphorus oxychloride and 5.0 ml (0.031 mol) of N,N-diethylaniline is heated at 115° C. for 3 hours. The dark, homogeneous mixture obtained is hydrolysed by adding crushed ice, the temperature being kept below 30° C. Extraction with diethyl ether, drying of the combined organic ethereal phases over sodium sulfate, and purification on a silica gel column (eluant: ethyl acetate/n-hexane 1/9) yields the desired target compound in a yield of 1.3 g (58% of theory).

Further purification by means of sublimation at 80-85° C./15 Torr yields the desired title compound, having a melting point of 63-64° C.

¹H NMR (300 MHz, CDCl₃): 8.635 ppm (s, 1H); 8.321 ppm (s, 1H); 4.025 ppm (s, 3H).

### Example P4

### Preparation of 5-methoxy-4-prop-2-ynyloxy-pyrimidine

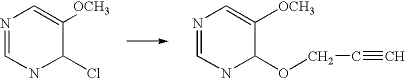

0.59 ml (0.01 mol) of propargyl alcohol is added to a suspension of 0.265 g (0.011 mol) of sodium hydride in 8 ml of N,N-dimethylformamide whilst cooling with ice-water. The reaction mixture is stirred at 20° C. for 30 minutes and then 1.44 g (0.01 mol) of 4-chloro-5-methoxy-pyrimidine (Example P3) at 0° C. are added and stirring is carried out at 20° C. for a further 3 hours. The reaction is stopped by adding ethyl acetate and water, and working-up is carried out. Purification of the resulting crude product on a silica gel column (eluant: ethyl acetate/isohexane 1/3) yields the desired target compound, having a melting point of 86-87° C., in a yield of 1.3 g (79% of theory).

¹H NMR (300 MHz, CDCl₃): 8.436 ppm (s, 1H); 8.101 ppm (s, 1H); 5.090 ppm (d, 2H); 3.945 ppm (s, 3H); 2.510 ppm (dxd, 1H).

### Example P5

### Preparation of {3-[3-(5-methoxy-pyrimidin-4-yloxy)-prop-1-ynyl]-phenyl}-acetonitrile

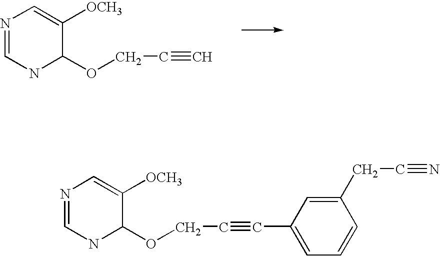

0.493 g (0.003 mol) of 5-methoxy-4-prop-2-ynyloxy-pyrimidine (Example P4) and 1.094 g (0.0045 mol) of 3-iodophenylacetonitrile are dissolved in separate amounts, each of 4 ml, of piperidine. Then, to the solution of 3-iodophenylacetonitrile, there are added first 0.175 g (0.00015 mol) of tetrakis(triphenylphosphine)palladium (Pd(PPh₃)₄) and also, after 5 minutes, the piperidine solution containing 5-methoxy-4-prop-2-ynyloxy-pyrimidine. 0.060 g (0.0003 mol) of copper(I) iodide (CuI) is added to the combined solution, whilst cooling with ice, and the reaction mixture is stirred at 20° C. for 3 hours. Working-up of the reaction mixture is starting by adding 25 ml of saturated aqueous ammonium chloride solution and 25 ml of ethyl acetate. The aqueous phase is extracted twice with ethyl acetate, and the combined organic phases are washed with saturated sodium chloride solution and dried over sodium sulfate. After filtering and concentrating by evaporation, 1.3 g of a yellow oil are obtained, which is purified by silica gel chromatography (eluant: ethyl acetate/isohexane 1/1). The desired target compound, having a melting point of 96-97° C., is obtained in a yield of 0.75 g (89% of theory).

¹H NMR (300 MHz, CDCl₃): 8.457 ppm (s, 1H); 8.116 ppm (s, 1H); 7.282-7.434 ppm (m, 4H); 5.318 ppm (s, 2H); 3.964 ppm (s, 3H); 3.724 ppm (s, 2H).

### Example P6

### Preparation of 2-methoxy-pyrazine 4-oxide (see J. Org. Chem. 1963 (28), 1682)

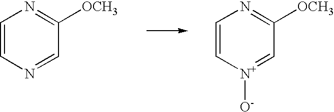

1.9 g (0.017 mol) of 30% hydrogen peroxide are added to a solution of 1.1 g (0.01 mol) of 2-methoxy-pyrazine in 3 ml of glacial acetic acid and the resulting solution is heated at 65-68° C. for 17 hours. The solution is concentrated to 1/3 of the original volume, diluted with the same amount of water and concentrated again. The residue is extracted with chloroform and the combined organic phases are washed with saturated sodium chloride solution and dried over sodium sulfate. After concentration, two amounts, each of 25 ml, of toluene are added to the residue obtained and are concentrated again. The desired title compound, which can be used directly for further reactions, is obtained in a yield of 0.98 g (77% of theory).

¹H NMR (300 MHz, CDCl₃): 7.988 ppm (d (J=4.2 Hz), 1H); 7.706-7.773 ppm (m, 1H); 7.745 ppm (dxd (J=4.2 and 1.5 Hz), 1H); 3.995 ppm (s, 3H).

### Example P7

### Preparation of 2-chloro-6-methoxy-pyrazine (see J. Heterocycl. Chem. 1994 (31), 1177)

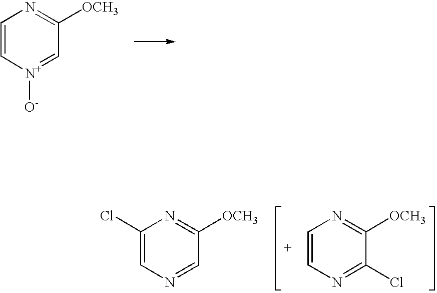

A mixture of 0.98 g (0.0078 mol) of 2-methoxy-pyrazine 4-oxide (Example P6) in 4 ml of phosphoryl chloride is stirred for 2 hours whilst heating at reflux, with 1.25 ml (0.0078 mol) of N,N-diethylaniline being metered in with the aid of a syringe before the start of the reaction. The resulting solution is cooled to 20° C. and poured onto ice-water. After adjusting the mixture to pH 9 with 30% aqueous sodium hydroxide solution, extraction is carried out four times, using 10 ml of chloroform each time. The combined extracts are washed with 3N hydrochloric acid and saturated sodium chloride solution and are dried over sodium sulfate. A mixture of the desired target compound and of the isomeric 2-chloro-3-methoxy-pyrazine is obtained, which can be separated on a silica gel column (eluant: ethyl acetate/isohexane 1/8). The desired title compound is obtained in a yield of 0.29 g (25% of theory), and the isomeric 2-chloro-3-methoxy-pyrazine in a yield of 0.38 g (33% of theory).

¹H NMR (300 MHz, CDCl₃) of the title compound: 8.143 ppm (s, 1H); 8.131 ppm (s, 1H); 3.988 ppm (s, 3H).

¹H NMR (300 MHz, CDCl₃) of 2-chloro-3-methoxy-pyrazine: 8.031 ppm (d (J=2.7 Hz), 1H); 7.937 ppm (d (J=2.7 Hz), 1H); 4.057 ppm (s, 3H).

### Example P8

### Preparation of 2-chloro-6-methoxy-pyrazine 4-oxide

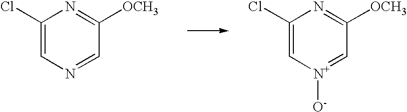

To a solution of 0.49 g (0.0034 mol) of 2-chloro-6-methoxy-pyrazine (Example P7) in 9 ml of dichloromethane there are added, under an argon gas atmosphere, first, within a period of 45 minutes and at 20° C., 0.19 g (0.002 mol) of hydrogen peroxide-urea adduct and then, dropwise, 0.3 ml (0.0021 mol) of trifluoroacetic anhydride in dichloromethane. Then there are again added, within a period of 45 minutes and at 20° C., 0.19 g (0.002 mol) of hydrogen peroxide-urea adduct and 0.3 ml (0.0021 mol) of trifluoroacetic anhydride in dichloromethane, and the reaction mixture is stirred further at 20° C. for 30 minutes. The reaction is stopped by adding water, and the aqueous phase obtained is extracted with chloroform. The combined organic phases are washed with 5% sodium hydrogen carbonate solution and saturated sodium chloride solution and dried over sodium sulfate. The desired title compound, having a melting point of 121-123° C., is obtained in a yield of 0.53 g (98% of theory). The compound can be used directly for the next reaction step.

¹H NMR (300 MHz, CDCl₃): 7.802 ppm (d (J=1.2 Hz), 1H); 7.691 ppm (d (J=1.2 Hz), 1H); 4.012 ppm (s, 3H).

### Example P9

### Preparation of 2,5-dichloro-3-methoxy-pyrazine (see J. Heterocycl. Chem. 1982 (19), 1061)

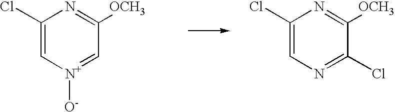

A mixture of 0.53 g (0.0033 mol) of 2-chloro-6-methoxy-pyrazine 4-oxide (Example P8) and 3 ml of phosphoryl chloride is boiled at reflux for 90 minutes. The excess of phosphoryl chloride is then removed under reduced pressure and the reaction mixture is poured onto ice-water and extracted with chloroform. The organic phase is washed with 5% sodium hydrogen carbonate solution and dried over sodium sulfate. After removal of the solvent by distillation, the crude product is obtained, which is purified on a silica gel column (eluant: ethyl acetate/isohexane 1/9). The desired target compound is obtained in a yield of 0.55 g (92% of theory).

¹H NMR (300 MHz, CDCl₃): 7.953 ppm (s, 1H); 4.078 ppm (s, 3H).

### Example P10

### Preparation of 5-chloro-3-methoxy-2-prop-2-ynyloxy-pyrazine

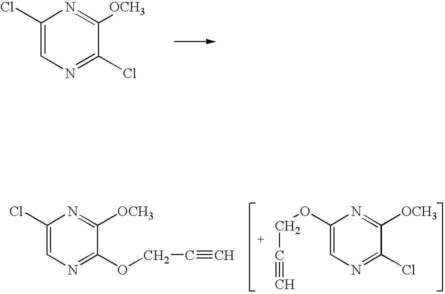

0.23 ml (0.0038 mol) of propargyl alcohol is added to a suspension of 0.155 g (0.0038 mol) of sodium hydride (60% suspension in oil) in 5 ml of N,N-dimethylformamide, whilst cooling with ice-water, and the resulting mixture is stirred at 20° C. for 30 minutes. Then, 0.55 g (0.003 mol) of 2,5-dichloro-3-methoxy-pyrazine (Example P9) is added, whilst cooling in an ice-water bath, and the mixture is stirred further at 20° C. for 3 hours. The reaction is stopped by adding ethyl acetate and water, and the aqueous phase is extracted with ethyl acetate. The combined organic phases are washed with saturated sodium chloride solution and dried over sodium sulfate. The crude product obtained is purified on a silica gel column (eluant: ethyl acetate/isohexane 1/9). The desired title compound, having a melting point of 71-72° C., is obtained in a yield of 0.23 g (53% of theory), and 0.07 g (17% of theory) of the isomeric 2-chloro-3-methoxy-5-prop-2-ynyloxy-pyrazine is obtained.

¹H NMR (300 MHz, CDCl₃) of the desired title compound: 7.650, ppm (s, 1H); 5.007 ppm (d (J=2.4 Hz), 2H); 4.043 ppm (s, 3H); 2.496 ppm (dxd (J=2.4 Hz), 1H).

¹H NMR (300 MHz, CDCl₃) of the isomeric 2-chloro-3-methoxy-5-prop-2-ynyloxy-pyrazine: 7.649 ppm (s, 1H); 4.953 ppm (d (J=2.4 Hz), 2H); 4.057 ppm (s, 3H); 2.493 ppm (dxd (J=2.4 Hz), 1H).

### Example P11

### Preparation of {3-[3-(5-chloro-3-methoxy-pyrazin-2-yloxy)-prop-1-ynyl]-phenyl}-acetonitrile

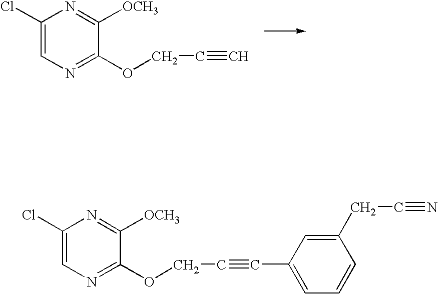

To a solution of 0.33 g (0.0016 mol) of 5-chloro-3-methoxy-2-prop-2-ynyloxy-pyrazine (Example P10) and 0.60 g (0.0025 mol) of 3-iodophenylacetonitrile in 3 ml of N,N-dimethyl-formamide and 1.5 ml of diisopropylethylamine there is added a mixture of 0.03 g of bis(triphenylphosphine)palladium dichloride (PdCl₂(PPh₃)₂), 0.03 g of triphenylphosphine (PPh₃) and 0.03 g of copper(I) iodide (CuI) at 20° C. under an argon gas atmosphere. The reaction mixture is stirred at 20° C. for 90 minutes. The reaction is stopped by adding ice, and the aqueous phase is extracted three times, using 10 ml of ethyl acetate each time. The combined organic phases are washed with saturated sodium chloride solution, dried over sodium sulfate, filtered and concentrated. The crude product obtained is purified on a silica gel column (eluant: ethyl acetate/isohexane 1/3). 0.20 g (38% of theory) of the desired title compound, having a melting point of 99-100° C., is obtained.

¹H NMR (300 MHz, CDCl₃): 7.671 ppm (s, 1H); 7.278-7.425 ppm (m, 4H); 5.233 ppm (s, 2H); 4.060 ppm (s, 3H); 3.721 ppm (s, 2H).

It is also possible for the preferred compounds listed in the following Tables to be obtained in a manner analogous to that described in Examples P1 to P11 or by the methods shown in Reaction Schemes 1-4 and in the references indicated. In the column “Phys. data”, the indicated temperatures denote the melting point (m.p.) of the compounds in question.

TABLE 1
Compounds of formula Ia1:

| | |
|---|---|
| | (Ia1) |
| 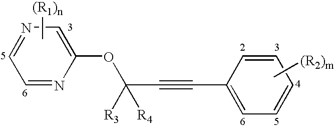 | |

| Comp. | | | | | Phys. data |
|---|---|---|---|---|---|
| no. | R₁ | R₂ | R₃ | R₄ | m.p. (° C.) |
| 1.001 | 3-OCH₃, 5-CN | 3-Cl | H | H | |
| 1.002 | 3-F, 5-Cl | 3-Cl | H | H | |
| 1.003 | 3-Cl, 5-Cl | 3-Cl | H | H | |
| 1.004 | 3-OCH₃, 5-F | 3-Cl | H | H | |
| 1.005 | 3-OCH₃, 5-Cl | 3-Cl | H | H | |
| 1.006 | 3-OCH₃, 5-Br | 3-Cl | H | H | |
| 1.007 | 3-Cl, 5-CF₃ | 3-Cl | H | H | |
| 1.008 | 3-OCH₃, 5-CF₃ | 3-Cl | H | H | |
| 1.009 | 3-OCH₃, 5-CH₃ | 3-Cl | H | H | |
| 1.010 | 3-OCH₃, 5-CH═NOCH₃ | 3-Cl | H | H | |
| 1.011 | 3-F, 5-CF₃ | 3-Cl | H | H | |
| 1.012 | 3-OCH₃ | 3-Cl | H | H | |
| 1.013 | 3-OCH₃, 5-CN | 3-CH₂CN, 4-F | H | H | |
| 1.014 | 3-F, 5-Cl | 3-CH₂CN, 4-F | H | H | |
| 1.015 | 3-Cl, 5-Cl | 3-CH₂CN, 4-F | H | H | |
| 1.016 | 3-OCH₃, 5-F | 3-CH₂CN, 4-F | H | H | |
| 1.017 | 3-OCH₃, 5-Cl | 3-CH₂CN, 4-F | H | H | |
| 1.018 | 3-OCH₃, 5-Br | 3-CH₂CN, 4-F | H | H | |
| 1.019 | 3-Cl, 5-CF₃ | 3-CH₂CN, 4-F | H | H | |
| 1.020 | 3-OH₃, 5-CF₃ | 3-CH₂CN, 4-F | H | H | |
| 1.021 | 3-OCH₃, 5-CH₃ | 3-CH₂CN, 4-F | H | H | |
| 1.022 | 3-OCH₃, 5-CH═NOCH₃ | 3-CH₂CN, 4-F | H | H | |
| 1.023 | 3-F, 5-CF₃ | 3-CH₂CN, 4-F | H | H | |
| 1.024 | 3-OCH₃ | 3-CH₂CN, 4-F | H | H | |
| 1.025 | 3-OCH₃, 5-CN | 3-CH(CH₃)CN | H | H | |
| 1.026 | 3-F, 5-Cl | 3-CH(CH₃)CN | H | H | |
| 1.027 | 3-Cl, 5-Cl | 3-CH(CH₃)CN | H | H | |
| 1.028 | 3-OCH₃, 5-F | 3-CH(CH₃)CN | H | H | |
| 1.029 | 3-OCH₃, 5-Cl | 3-CH(CH₃)CN | H | H | |
| 1.030 | 3-OCH₃, 5-Br | 3-CH(CH₃)CN | H | H | |
| 1.031 | 3-Cl, 5-CF₃ | 3-CH(CH₃)CN | H | H | |
| 1.032 | 3-OCH₃, 5-CF₃ | 3-CH(CH₃)CN | H | H | |
| 1.033 | 3-OCH₃, 5-CH₃ | 3-CH(CH₃)CN | H | H | |
| 1.034 | 3-OCH₃, 5-CH═NOCH₃ | 3-CH(CH₃)CN | H | H | |
| 1.035 | 3-F, 5-CF₃ | 3-CH(CH₃)CN | H | H | |
| 1.036 | 3-OCH₃ | 3-CH(CH₃)CN | H | H | |
| 1.037 | 3-OCH₃, 5-CN | 3-CH₂CN | H | H | |
| 1.038 | 3-F, 5-Cl | 3-CH₂CN | H | H | |
| 1.039 | 3-Cl, 5-Cl | 3-CH₂CN | H | H | |
| 1.040 | 3-OCH₃, 5-F | 3-CH₂CN | H | H | |
| 1.041 | 3-OCH₃, 5-Cl | 3-CH₂CN | H | H | crystalline |
| 1.042 | 3-OCH₃, 5-Br | 3-CH₂CN | H | H | |
| 1.043 | 3-Cl, 5-CF₃ | 3-CH₂CN | H | H | |
| 1.044 | 3-OCH₃, 5-CF₃ | 3-CH₂CN | H | H | |
| 1.045 | 3-OCH₃, 5-CH₃ | 3-CH₂CN | H | H | |
| 1.046 | 3-OCH₃, 5-CH═NOCH₃ | 3-CH₂CN | H | H | |
| 1.047 | 3-F, 5-CF₃ | 3-CH₂CN | H | H | |
| 1.048 | 3-OCH₃ | 3-CH₂CN | H | H | 104-105 |
| 1.049 | 3-OCH₃, 5-F | 3-CH₂CN | CH₃ | H | |
| 1.050 | 3-OCH₃, 5-Cl | 3-CH₂CN | CH₃ | H | |
| 1.051 | 3-OCH₃, 5-CF₃ | 3-CH₂CN | CH₃ | H | |
| 1.052 | 3-OCH₃, 5-Br | 3-CH₂CN | CH₃ | H | |
| 1.053 | 3-Cl, 5-CF₃ | 3-CH₂CN | CH₃ | H | |
| 1.054 | 3-OCH₃, 5-CN | 3-CH₂CN | CH₃ | H | |
| 1.055 | 3-OCH₃, 5-F | 3-CHF₂ | H | H | |
| 1.056 | 3-OCH₃, 5-Cl | 3-OH F2 | H | H | |
| 1.057 | 3-OCH₃, 5-CF₃ | 3-CHF₂ | H | H | |
| 1.058 | 3-OCH₃, 5-Br | 3-CHF₂ | H | H | |
| 1.059 | 3-Cl, 5-CF₃ | 3-CHF₂ | H | H | |
| 1.060 | 3-OCH₃, 5-CN | 3-CHF₂ | H | H | |
| 1.061 | 3-OCH₃, 5-F | 3-CH₃ | H | H | |
| 1.062 | 3-OCH₃, 5-Cl | 3-CH₃ | H | H | |
| 1.063 | 3-OCH₃, 5-CF₃ | H | H | | |
| 1.064 | 3-OCH₃, 5-Br | 3-CH₃ | H | H | |
| 1.065 | 3-Cl, 5-CF₃ | 3-CH₃ | H | H | |
| 1.066 | 3-OCH₃, 5-CN | 3-CH₃ | H | H | |
| 1.067 | 3-OCH₃, 5-F | 3-CH₂—CN | F | F | |
| 1.068 | 3-OCH₃, 5-Cl | 3-CH₂—CN | F | F | |
| 1.069 | 3-OCH₃, 5-CF₃ | 3-CH₂—CN | F | F | |
| 1.070 | 3-OCH₃, 5-Br | 3-CH₂—CN | F | F | |
| 1.071 | 3-Cl, 5-CF₃ | 3-CH₂—CN | F | F | |
| 1.072 | 3-OCH₃, 5-CN | 3-CH₂—CN | F | F | |
| 1.073 | 5-OCH₃, 6-F | 3-CH₂—CN | H | H | |
| 1.074 | 5-OCH₃, 6-Cl | 3-CH₂—CN | H | H | crystalline |
| 1.075 | 5-OCH₃, 6-F | 3-CH(CH₃)CN | H | H | |
| 1.076 | 5-OCH₃, 6-Cl | 3-CH(CH₃)CN | H | H | |
| 1.077 | 5-OCH₃, 6-Cl | 3-CHF₂ | H | H | |
| 1.078 | 6-OCH₃ | 3-CH₂—CN | H | H | 71-72 |
| 1.079 | 5,6-CH═CH—CH═CH— | 3-CH₂—CN | H | H | amorphous |
| 1.080 | 5,6-CH═CCl—CH═CH— | 3-CH₂—CN | H | H | solid |
| 1.081 | 5,6-CH═CH—CH═CH—, | 3-CH₂—CN | H | H | solid |
| | 3-OCH₃ | | | | |
| 1.082 | 5,6-CH═CCl—CH═CH—, | 3-CH₂—CN | H | H | crystalline |
| | 3-OCH₃ | | | | |
| 1.083 | 5,6-CH═CCl—CH═CH—, | 4-Cl | H | H | crystalline |
| | 3-OCH₃ | | | | |
| 1.084 | 5-Cl, 6-OCH₃ | 3-CH₂—CN | H | H | crystalline |
| 1.085 | 5,6-CH═CH—CCl═CH—, | 3-CH₂—CN | H | H | crystalline |
| | 3-Cl | | | | |
| 1.086 | 5,6-CCl═CH—CH═CH—, | 3-CH₂—CN | H | H | crystalline |
| | 3-Cl | | | | |
| 1.087 | 3-Cl | 3-CH₂—CN | H | H | crystalline |
| 1.088 | 5,6-CH═CCl—CH═CH—, | 3-CH₂—CN | H | H | |
| | 3-N-piperidyl | | | | |
| 1.089 | 5,6-CH═CH—CH═CH—, | 4-Cl | H | H | |
| | 3-(2-thienyl) | | | | |
| 1.090 | 5,6-CH═CH—CH═CH—, | 4-Cl | H | H | |
| | 3-phenyl | | | | |
| 1.091 | 3-OCH₃, 5-Cl | 3-CH₂CN | H | H | |
| 1.092 | 3-OCH₃, 5-Cl | 3-CH(CH₃)CN | H | H | |
| 1.093 | 3-OCH₃, 5-Cl | 3-Cl | H | H | |
| 1.094 | 3-OCH₃, 5-Cl | 3-CH₂—CN, 4-F | H | H | |
| 1.095 | 3-OCH₃, 5-Cl | 3-CH(CH₃)CN, 4-F | H | H | |
| 1.096 | 3-OCH₃, 5-Cl | 3-CH₂—CN, 6-F | H | H | |
| 1.097 | 3-OCH₃, 5-Cl | 3-CH(CH₃)CN, 6-F | H | H | |
| 1.098 | 3-OCH₃, 5-Cl | 3-CH₂—CN, 4-F, 6-F | H | H | |
| 1.099 | 3-OCH₃, 5-Cl | 3-CH(CH₃)CN, 4-F, 6-F | H | H | |
| 1.100 | 3-OCH₃, 5-Cl | 3-CH₂—CN, 4-Cl, 6-F | H | H | |
| 1.101 | 3-OCH₃, 5-Cl | 3-CH(CH₃)CN, 4-Cl, 6-F | H | H | |
| 1.102 | 3-OCH₃, 5-Cl | 3-Cl, 6-F | H | H | |
| 1.103 | 3-OCH₃, 5-Cl | 3-CH₂—CN, 4-CN | H | H | |
| 1.104 | 3-OCH₃, 5-Cl | 3-CH₂—CN, 4-CH₃ | H | H | |
| 1.105 | 3-OCH₃, 5-Cl | 3-CH(CH₃)CN, 4-CH₃ | H | H | |
| 1.106 | 3-OCH₃, 5-Cl | 3-Br | H | H | |
| 1.107 | 3-OCH₃, 5-Cl | 3-I | H | H | |
| 1.108 | 3-OCH₃, 5-Cl | 3-I, 6-F | H | H | |
| 1.109 | 3-OCH₃, 5-Cl | 3-CH₂—CN, 4-NO₂ | H | H | |
| 1.110 | 3-OCH₃, 5-Cl | 3-CH₂—CN, 4-NH₂ | H | H | |
| 1.111 | 3-OCH₃, 5-Cl | 3-CHF₂ | H | H | |
| 1.112 | 3-OCH₃, 5-Cl | 3-CHF₂, 4-F | H | H | |
| 1.113 | 3-OCH₃, 5-Cl | 3-CHF₂, 6-F | H | H | |
| 1.114 | 3-OCH₃, 5-Cl | 3-CHF₂, 4-F, 6-F | H | H | |
| 1.115 | 3-OCH₃, 5-CH═NOCH₃ | 3-CH₂CN | H | H | |
| 1.116 | 3-OCH₃, 5-CH═NOCH₃ | 3-CH(CH₃)CN | H | H | |
| 1.117 | 3-OCH₃, 5-CH═NOCH₃ | 3-Cl | H | H | |
| 1.118 | 3-OCH₃, 5-CH═NOCH₃ | 3-CH₂—CN, 4-F | H | H | |
| 1.119 | 3-OCH₃, 5-CH═NOCH₃ | 3-CH(CH₃)CN, 4-F | H | H | |
| 1.120 | 3-OCH₃, 5-CH═NOCH₃ | 3-CH₂—CN, 6-F | H | H | |
| 1.121 | 3-OCH₃, 5-CH═NOCH₃ | 3-CH(CH₃)CN, 6-F | H | H | |
| 1.122 | 3-OCH₃, 5-CH═NOCH₃ | 3-CH₂—CN, 4-F, 6-F | H | H | |
| 1.123 | 3-OCH₃, 5-CH═NOCH₃ | 3-CH(CH₃)CN, 4-F, 6-F | H | H | |
| 1.124 | 3-OCH₃, 5-CH═NOCH₃ | 3-CH₂—CN, 4-Cl, 6-F | H | H | |
| 1.125 | 3-OCH₃, 5-CH═NOCH₃ | 3-CH(CH₃)CN, 4-Cl, 6-F | H | H | |
| 1.126 | 3-OCH₃, 5-CH═NOCH₃ | 3-Cl, 6-F | H | H | |
| 1.127 | 3-OCH₃, 5-CH═NOCH₃ | 3-CH₂—CN, 4-CN | H | H | |
| 1.128 | 3-OCH₃, 5-CH═NOCH₃ | 3-CH₂—CN, 4-CH₃ | H | H | |
| 1.129 | 3-OCH₃, 5-CH═NOCH₃ | 3-CH(CH₃)CN, 4-CH₃ | H | H | |
| 1.130 | 3-OCH₃, 5-CH═NOCH₃ | 3-Br | H | H | |
| 1.131 | 3-OCH₃, 5-CH═NOCH₃ | 3-I | H | H | |
| 1.132 | 3-OCH₃, 5-CH═NOCH₃ | 3-I, 6-F | H | H | |
| 1.133 | 3-OCH₃, 5-CH═NOCH₃ | 3-CH₂—CN, 4-NO₂ | H | H | |
| 1.134 | 3-OCH₃, 5-CH═NOCH₃ | 3-CH₂—CN, 4-NH₂ | H | H | |
| 1.135 | 3-OCH₃, 5-CH═NOCH₃ | 3-CHF₂ | H | H | |
| 1.136 | 3-OCH₃, 5-CH═NOCH₃ | 3-CHF₂, 4-F | H | H | |
| 1.137 | 3-OCH₃, 5-CH═NOCH₃ | 3-CHF₂, 6-F | H | H | |
| 1.138 | 3-OCH₃, 5-CH═NOCH₃ | 3-CHF₂, 4-F, 6-F | H | H | |
| 1.139 | 3-OCH₃, 5-Cl | 3-CHF₂, 4-Cl, 6-F | H | H | |
| 1.140 | 3-OCH₃, 5-Cl | 3-CHF₂, 4-CH₃, 6-F | H | H | |
| 1.141 | 3-OCH₃, 5-Cl | 3-CHF₂, 4-CN, 6-F | H | H | |
| 1.142 | 3-OCH₃, 5-CF₃ | 3-CH₂CN, 4-Cl, 6-F | H | H | |
| 1.143 | 3-OCH₃, 5-CF₃ | 3-CH₂CN, 4-CH₃, 6-F | H | H | |
| 1.144 | 3-OCH₃, 5-CF₃ | 3-CH₂CN, 4-CN, 6-F | H | H | |
| 1.145 | 3-OCH₃ | 3-CH₂CN, 6-F | H | H | |
| 1.146 | 3-OCH₃ | 3-CH₂CN, 4-F | H | H | |
| 1.147 | 3-OCH₃ | 3-CHF₂, 6-F | H | H | |
| 1.148 | 3-OCH₃ | 3-CHF₂, 4-F | H | H | |
| 1.149 | 3-OCH₃ | 3-CH(CH₃)CN | H | H | |
| 1.150 | 3-OCH₃ | 3-CH₂CN, 4-F | H | H | |
| 1.151 | 3-OCH₃ | 3-CH(CH₃)CN, 4-F | H | H | |
| 1.152 | 3-OCH₃ | 3-CH₂CN, 6-F | H | H | |
| 1.153 | 3-OCH₃ | 3-CH(CH₃)CN, 6-F | H | H | |
| 1.154 | 3-OCH₃ | 3-CH₂CN, 4-F, 6-F | H | H | |
| 1.155 | 3-OCH₃ | 3-CH(CH₃)CN, 4-F, 6-F | H | H | |
| 1.156 | 3-OCH₃ | 3-CH₂CN, 4-Cl, 6-F | H | H | |
| 1.157 | 3-OCH₃ | 3-CH(CH₃)CN, 4-Cl, 6-F | H | H | |
| 1.158 | 3-OCH₃ | 3-Cl, 6-F | H | H | |
| 1.159 | 3-OCH₃ | 3-CH₂CN, 4-CN | H | H | |
| 1.160 | 3-OCH₃ | 3-CH₂CN, 4-CH₃ | H | H | |
| 1.161 | 3-OCH₃ | 3-OH(CH₃)CN, 4-CH₃ | H | H | |
| 1.162 | 3-OCH₃ | 3-Br | H | H | |
| 1.163 | 3-OCH₃ | 3-I | H | H | |
| 1.164 | 3-OCH₃ | 3-I, 6-F | H | H | |
| 1.165 | 3-OCH₃ | 3-CH₂CN, 4-NO₂ | H | H | |
| 1.166 | 3-OCH₃ | 3-CH₂CN, 4-NH₂ | H | H | |
| 1.167 | 3-OCH₃ | 3-CHF₂ | H | H | |
| 1.168 | 3-OCH₃ | 3-CHF₂, 4-F | H | H | |
| 1.169 | 3-OCH₃ | 3-CHF₂, 6-F | H | H | |
| 1.170 | 3-OCH₃ | 3-CHF₂, 4-F, 6-F | H | H | |
| 1.171 | 3-OCH₃, 5-F | 3-CH₂CN, 4-F | H | H | |
| 1.172 | 3-OCH₃, 5-F | 3-CH(CH₃)CN, 4-F | H | H | |
| 1.173 | 3-OCH₃, 5-F | 3-CH₂CN, 6-F | H | H | |
| 1.174 | 3-OCH₃, 5-F | 3-CH(CH₃)CN, 6-F | H | H | |
| 1.175 | 3-OCH₃, 5-F | 3-CH₂CN, 4-F, 6-F | H | H | |
| 1.176 | 3-OCH₃, 5-F | 3-CH(CH₃)CN, 4-F, 6-F | H | H | |
| 1.177 | 3-OCH₃, 5-F | 3-CH₂CN, 4-Cl, 6-F | H | H | |
| 1.178 | 3-OCH₃, 5-F | 3-CH(CH₃)CN, 4-Cl, 6-F | H | H | |
| 1.179 | 3-OCH₃, 5-F | 3-CH₂CN, 4-CN | H | H | |
| 1.180 | 3-OCH₃, 5-F | 3-CH₂CN, 4-CH₃ | H | H | |
| 1.181 | 3-OCH₃, 5-F | 3-CH(CH₃)CN, 4-CH₃ | H | H | |
| 1.182 | 3-OCH₃, 5-F | 3-CH₂CN, 4-NO₂ | H | H | |
| 1.183 | 3-OCH₃, 5-F | 3-CH₂CN, 4-NH₂ | H | H | |
| 1.184 | 3-OCH₃, 5-F | 3-CHF₂, 4-F | H | H | |
| 1.185 | 3-OCH₃, 5-F | 3-CHF₂, 6-F | H | H | |
| 1.186 | 3-OCH₃, 5-F | 3-CHF₂, 4-F, 6-F | H | H | |

TABLE 2
Compounds of formula Ia3:

| | |
|---|---|
| | (Ia3) |
| 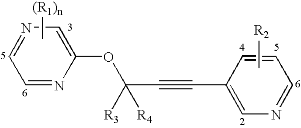 | |

| | | | | | Phys. data |
|---|---|---|---|---|---|
| Comp. | | | | | m.p. |
| no. | R₁ | R₂ | R₃ | R₄ | (° C.) |
| 2.001 | 3-OCH₃, 5-CN | 6-CH₃ | H | H | |
| 2.002 | 3-F, 5-Cl | 6-CH₃ | H | H | |
| 2.003 | 3-Cl, 5-Cl | 6-CH₃ | H | H | |
| 2.004 | 3-OCH₃, 5-F | 6-CH₃ | H | H | |
| 2.005 | 3-OCH₃, 5-Cl | 6-CH₃ | H | H | |
| 2.006 | 3-OCH₃, 5-Br | 6-CH₃ | H | H | |
| 2.007 | 3-Cl, 5-CF₃ | 6-CH₃ | H | H | |
| 2.008 | 3-OCH₃, 5-CF₃ | 6-CH₃ | H | H | |
| 2.009 | 3-OCH₃, 5-CH₃ | 6-CH₃ | H | H | |
| 2.010 | 3-OCH₃, 5-CH═NOCH₃ | 6-CH₃ | H | H | |
| 2.011 | 3-F, 5-CF₃ | 6-CH₃ | H | H | |
| 2.012 | 3-OCH₃ | 6-CH₃ | H | H | |
| 2.013 | 3-OCH₃, 5-CN | 6-Cl | H | H | |
| 2.014 | 3-F, 5-Cl | 6-Cl | H | H | |
| 2.015 | 3-Cl, 5-Cl | 6-Cl | H | H | |
| 2.016 | 3-OCH₃, 5-F | 6-Cl | H | H | |
| 2.017 | 3-OCH₃, 5-Cl | 6-Cl | H | H | |
| 2.018 | 3-OCH₃, 5-Br | 6-Cl | H | H | |
| 2.019 | 3-Cl, 5-CF₃ | 6-Cl | H | H | |
| 2.020 | 3-OCH₃, 5-CF₃ | 6-Cl | H | H | |
| 2.021 | 3-OCH₃, 5-CH₃ | 6-Cl | H | H | |
| 2.022 | 3-OCH₃, 5-CH═NOCH₃ | 6-Cl | H | H | |
| 2.023 | 3-F, 5-CF₃ | 6-Cl | H | H | |
| 2.024 | 3-OCH₃ | 6-Cl | H | H | |
| 2.025 | 3-OCH₃, 5-CN | 5-CH₂CN | H | H | |
| 2.026 | 3-F, 5-Cl | 5-CH₂CN | H | H | |
| 2.027 | 3-Cl, 5-Cl | 5-CH₂CN | H | H | |
| 2.028 | 3-OCH₃, 5-F | 5-CH₂CN | H | H | |
| 2.029 | 3-OCH₃, 5-Cl | 5-CH₂CN | H | H | |
| 2.030 | 3-OCH₃, 5-Br | 5-CH₂CN | H | H | |
| 2.031 | 3-Cl, 5-CF₃ | 5-CH₂CN | H | H | |
| 2.032 | 3-OCH₃, 5-CF₃ | 5-CH₂CN | H | H | |
| 2.033 | 3-OCH₃, 5-CH₃ | 5-CH₂CN | H | H | |
| 2.034 | 3-OCH₃, 5-CH═NOCH₃ | 5-CH₂CN | H | H | |
| 2.035 | 3-F, 5-CF₃ | 5-CH₂CN | H | H | |
| 2.036 | 3-OCH₃ | 5-CH₂CN | H | H | |
| 2.037 | 3-OCH₃, 5-CN | 5-CH(CH₃)CN | H | H | |
| 2.038 | 3-F, 5-Cl | 5-CH(CH₃)CN | H | H | |
| 2.039 | 3-Cl, 5-Cl | 5-CH(CH₃)CN | H | H | |
| 2.040 | 3-OCH₃, 5-F | 5-CH(CH₃)CN | H | H | |
| 2.041 | 3-OCH₃, 5-Cl | 5-CH(CH₃)CN | H | H | |
| 2.042 | 3-OCH₃, 5-Br | 5-CH(CH₃)CN | H | H | |
| 2.043 | 3-Cl, 5-CF₃ | 5-CH(CH₃)CN | H | H | |
| 2.044 | 3-OCH₃, 5-CF₃ | 5-CH(CH₃)CN | H | H | |
| 2.045 | 3-OCH₃, 5-CH₃ | 5-CH(CH₃)CN | H | H | |
| 2.046 | 3-OCH₃, 5-CH═NOCH₃ | 5-CH(CH₃)CN | H | H | |
| 2.047 | 3-F, 5-CF₃ | 5-CH(CH₃)CN | H | H | |
| 2.048 | 3-OCH₃ | 5-OH(CH₃)CN | H | H | |
| 2.049 | 3-OCH₃, 5-F | 5-CH₂—CN | F | F | |
| 2.050 | 3-OCH₃, 5-Cl | 5-CH₂—CN | F | F | |
| 2.051 | 3-OCH₃, 5-CF₃ | 5-CH₂—CN | F | F | |
| 2.052 | 3-OCH₃, 5-Br | 5-CH₂—CN | F | F | |
| 2.053 | 3-Cl, 5-CF₃ | 5-CH₂—CN | F | F | |
| 2.054 | 3-OCH₃, 5-CN | 5-CH₂—CN | F | F | |

TABLE 3
Compounds of formula Ia2:

| | |
|---|---|
| | (Ia2) |
| 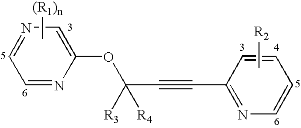 | |

| Comp. | | | | | Phys. data |
|---|---|---|---|---|---|
| no. | R₁ | R₂ | R₃ | R₄ | m.p. (° C.) |
| 3.001 | 3-OCH₃, 5-CN | 4-CH₃ | H | H | |
| 3.002 | 3-F, 5-Cl | 4-CH₃ | H | H | |
| 3.003 | 3-Cl, 5-Cl | 4-CH₃ | H | H | |
| 3.004 | 3-OCH₃, 5-F | 4-CH₃ | H | H | |
| 3.005 | 3-OCH₃, 5-Cl | 4-CH₃ | H | H | |
| 3.006 | 3-OCH₃, 5-Br | 4-CH₃ | H | H | |
| 3.007 | 3-Cl, 5-CF₃ | 4-CH₃ | H | H | |
| 3.008 | 3-OCH₃, 5-CF₃ | 4-CH₃ | H | H | |
| 3.009 | 3-OCH₃, 5-CH₃ | 4-CH₃ | H | H | |
| 3.010 | 3-OCH₃, 5-CH═NOCH₃ | 4-CH₃ | H | H | |
| 3.011 | 3-F, 5-CF₃ | 4-CH₃ | H | H | |
| 3.012 | 3-OCH₃ | 4-CH₃ | H | H | |
| 3.013 | 3-OCH₃, 5-CN | 4-CH₂—CN | H | H | |
| 3.014 | 3-F, 5-Cl | 4-CH₂—CN | H | H | |
| 3.015 | 3-Cl, 5-Cl | 4-CH₂—CN | H | H | |
| 3.016 | 3-OCH₃, 5-F | 4-CH₂—CN | H | H | |
| 3.017 | 3-OCH₃, 5-Cl | 4-CH₂—CN | H | H | |
| 3.018 | 3-OCH₃, 5-Br | 4-CH₂—CN | H | H | |
| 3.019 | 3-Cl, 5-CF₃ | 4-CH₂—CN | H | H | |
| 3.020 | 3-OCH₃, 5-CF₃ | 4-CH₂—CN | H | H | |
| 3.021 | 3-OCH₃, 5-CH₃ | 4-CH₂—CN | H | H | |
| 3.022 | 3-OCH₃, 5-CH═NOCH₃ | 4-CH₂—CN | H | H | |
| 3.023 | 3-F, 5-CF₃ | 4-CH₂—CN | H | H | |
| 3.024 | 3-OCH₃ | 4-CH₂—CN | H | H | |
| 3.025 | 3-OCH₃, 5-F | 4-CH₂—CN | F | F | |
| 3.026 | 3-OCH₃, 5-Cl | 4-CH₂—CN | F | F | |
| 3.027 | 3-OCH₃, 5-CF₃ | 4-CH₂—CN | F | F | |
| 3.028 | 3-OCH₃, 5-Br | 4-CH₂—CN | F | F | |
| 3.029 | 3-Cl, 5-CF₃ | 4-CH₂—CN | F | F | |
| 3.030 | 3-OCH₃, 5-CN | 4-CH₂—CN | F | F | |
| 3.031 | 3-OCH₃, 5-F | 4-CHF₂ | H | H | |
| 3.032 | 3-OCH₃, 5-Cl | 4-CHF₂ | H | H | |
| 3.033 | 3-OCH₃, 5-CF₃ | 4-CHF₂ | H | H | |
| 3.034 | 3-OCH₃, 5-Br | 4-CHF₂ | H | H | |
| 3.035 | 3-Cl, 5-CF₃ | 4-CHF₂ | H | H | |
| 3.036 | 3-OCH₃, 5-CN | 4-CHF₂ | H | H | |

TABLE 4
Compounds of formula Ib₅1:

| | |
|---|---|
| | (Ib₅1) |
| 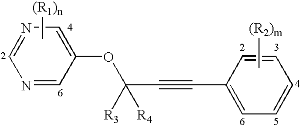 | |

| Comp. | | | | | Phys. data |
|---|---|---|---|---|---|
| no. | R₁ | R₂ | R₃ | R₄ | m.p. (° C.) |
| 4.001 | 2-CN; 4-OCH₃ | 3-CH₂—CN, 4-F | H | H | |
| 4.002 | 2-Cl, 4-F | 3-CH₂—CN, 4-F | H | H | |
| 4.003 | 2-Cl, 4-Cl | 3-CH₂—CN, 4-F | H | H | |
| 4.004 | 2-F, 4-OCH₃ | 3-CH₂—CN, 4-F | H | H | |
| 4.005 | 2-Cl, 4-OCH₃ | 3-CH₂—CN, 4-F | H | H | |
| 4.006 | 2-Br, 4-OCH₃ | 3-CH₂—CN, 4-F | H | H | |
| 4.007 | 2-CF₃, 4-Cl | 3-CH₂—CN, 4-F | H | H | |
| 4.008 | 2-CF₃, 4-OCH₃ | 3-CH₂—CN, 4-F | H | H | |
| 4.009 | 2-CH₃, 4-OCH₃ | 3-CH₂—CN, 4-F | H | H | |
| 4.010 | 2-CH═NOCH₃, 4-OCH₃ | 3-CH₂—CN, 4-F | H | H | |
| 4.011 | 2-CF₃, 4-F | 3-CH₂—CN, 4-F | H | H | |
| 4.012 | 4-OCH₃ | 3-CH₂—CN, 4-F | H | H | |
| 4.013 | 2-CN; 4-OCH₃ | 3-CH(CH₃)CN | H | H | |
| 4.014 | 2-Cl, 4-F | 3-CH(CH₃)CN | H | H | |
| 4.015 | 2-Cl, 4-Cl | 3-CH(CH₃)CN | H | H | |
| 4.016 | 2-F, 4-OCH₃ | 3-CH(CH₃)CN | H | H | |
| 4.017 | 2-Cl, 4-OCH₃ | 3-CH(CH₃)CN | H | H | |
| 4.018 | 2-Br, 4-OCH₃ | 3-CH(CH₃)CN | H | H | |
| 4.019 | 2-CF_{3, 4-Cl} | 3-CH(CH₃)CN | H | H | |
| 4.020 | 2-CF₃, 4-OCH₃ | 3-CH(CH₃)CN | H | H | |
| 4.021 | 2-CH₃, 4-OCH₃ | 3-CH(CH₃)CN | H | H | |
| 4.022 | 2-CH═NOCH₃, 4-OCH₃ | 3-CH(CH₃)CN | H | H | |
| 4.023 | 2-CF₃, 4-F | 3-CH(CH₃)CN | H | H | |
| 4.024 | 4-OCH₃ | 3-CH(CH₃)CN | H | H | |
| 4.025 | 2-CN; 4-OCH₃ | 3-CH₂—CN | H | H | |
| 4.026 | 2-Cl, 4-F | 3-CH₂—CN | H | H | |
| 4.027 | 2-Cl, 4-Cl | 3-CH₂—CN | H | H | |
| 4.028 | 2-F, 4-OCH₃ | 3-CH₂—CN | H | H | |
| 4.029 | 2-Cl, 4-OCH₃ | 3-CH₂—CN | H | H | crystalline |
| 4.030 | 2-Br, 4-OCH₃ | 3-CH₂—CN | H | H | |
| 4.031 | 2-CF₃, 4-Cl | 3-CH₂—CN | H | H | |
| 4.032 | 2-CF₃, 4-OCH₃ | 3-CH₂—CN | H | H | resin |
| 4.033 | 2-CH₃, 4-OCH₃ | 3-CH₂—CN | H | H | crystalline |
| 4.034 | 2-CH═NOCH₃, 4-OCH₃ | 3-CH₂—CN | H | H | |
| 4.035 | 2-CF₃, 4-F | 3-CH₂—CN | H | H | |
| 4.036 | 4-OCH₃ | 3-CH₂—CN | H | H | |
| 4.037 | 2-F, 4-OCH₃ | 3-CH₂—CN | F | F | |
| 4.038 | 2-Cl, 4-OCH₃ | 3-CH₂—CN | F | F | |
| 4.039 | 2-CF₃, 4-OCH₃ | 3-CH₂—CN | F | F | |
| 4.040 | 2-Br, 4-OCH₃ | 3-CH₂—CN | F | F | |
| 4.041 | 2-CF₃, 4-Cl | 3-CH₂—CN | F | F | |
| 4.042 | 2-CN; 4-OCH₃ | 3-CH₂—CN | F | F | |
| 4.043 | 2-NH₂, 4-OCH₃ | 3-CH₂—CN | H | H | crystalline |
| 4.044 | 2-SCH₃ | 3-CH₂—CN | H | H | 97-98 |
| 4.045 | 2-CF₃, 4-N(CH₃)₂ | 3-CH₂—CN | H | H | crystalline |
| 4.046 | 2-CH₃, 4-SCH₃ | 3-CH₂—CN | H | H | |
| 4.047 | 2-S(O)₂CH₃ | 4-Cl | H | H | |
| 4.048 | 2-CN; 4-OCH₃ | 3-CH₂—CN, 6-F | H | H | |
| 4.049 | 2-Cl, 4-F | 3-CH₂—CN, 6-F | H | H | |
| 4.050 | 2-Cl, 4-Cl | 3-CH₂—CN, 6-F | H | H | |
| 4.051 | 2-F, 4-OCH₃ | 3-CH₂—CN, 6-F | H | H | |
| 4.052 | 2-Cl, 4-OCH₃ | 3-CH₂—CN, 6-F | H | H | |
| 4.053 | 2-Br, 4-OCH₃ | 3-CH₂—CN, 6-F | H | H | |
| 4.054 | 2-CF₃, 4-Cl | 3-CH₂—CN, 6-F | H | H | |
| 4.055 | 2-CF₃, 4-OCH₃ | 3-CH₂—CN, 6-F | H | H | |
| 4.056 | 2-CH₃, 4-OCH₃ | 3-CH₂—CN, 6-F | H | H | |
| 4.057 | 2-CH═NOCH₃, 4-OCH₃ | 3-CH₂—CN, 6-F | H | H | |
| 4.058 | 2-CF₃, 4-F | 3-CH₂—CN, 6-F | H | H | |
| 4.059 | 4-OCH₃ | 3-CH₂—CN, 6-F | H | H | |
| 4.060 | 2-CN; 4-OCH₃ | 3-CH(CH₃)CN, 4-F | H | H | |
| 4.061 | 2-Cl, 4-F | 3-CH(CH₃)CN, 4-F | H | H | |
| 4.062 | 2-Cl, 4-Cl | 3-CH(CH₃)CN, 4-F | H | H | |
| 4.063 | 2-F, 4-OCH₃ | 3-CH(CH₃)CN, 4-F | H | H | |
| 4.064 | 2-Cl, 4-OCH₃ | 3-CH(CH₃)CN, 4-F | H | H | |
| 4.065 | 2-Br, 4-OCH₃ | 3-CH(CH₃)CN, 4-F | H | H | |
| 4.066 | 2-CF₃, 4-Cl | 3-CH(CH₃)CN, 4-F | H | H | |
| 4.067 | 2-CF₃, 4-OCH₃ | 3-CH(CH₃)CN, 4-F | H | H | |
| 4.068 | 2-CH₃, 4-OCH₃ | 3-CH(CH₃)CN, 4-F | H | H | |
| 4.069 | 2-CH═NOCH₃, 4-OCH₃ | 3-CH(CH₃)CN, 4-F | H | H | |
| 4.070 | 2-CF₃, 4-F | 3-CH(CH₃)CN, 4-F | H | H | |
| 4.071 | 4-OCH₃ | 3-CH(CH₃)CN, 4-F | H | H | |
| 4.072 | 2-CN; 4-OCH₃ | 3-CH₂—CN, 4-Cl, 6-F | H | H | |
| 4.073 | 2-Cl, 4-F | 3-CH₂—CN, 4-Cl, 6-F | H | H | |
| 4.074 | 2-Cl, 4-Cl | 3-CH₂—CN, 4-Cl, 6-F | H | H | |
| 4.075 | 2-F, 4-OCH₃ | 3-CH₂—CN, 4-Cl, 6-F | H | H | |
| 4.076 | 2-Cl, 4-OCH₃ | 3-CH₂—CN, 4-Cl, 6-F | H | H | |
| 4.077 | 2-Br, 4-OCH₃ | 3-CH₂—CN, 4-Cl, 6-F | H | H | |
| 4.078 | 2-CF₃, 4-Cl | 3-CH₂—CN, 4-Cl, 6-F | H | H | |
| 4.079 | 2-CF₃, 4-OCH₃ | 3-CH₂—CN, 4-Cl, 6-F | H | H | |
| 4.080 | 2-CH₃, 4-OCH₃ | 3-CH₂—CN, 4-Cl, 6-F | H | H | |
| 4.081 | 2-CH═NOCH₃, 4-OCH₃ | 3-CH₂—CN, 4-Cl, 6-F | H | H | |
| 4.082 | 2-CF₃, 4-F | 3-CH₂-CN, 4-Cl, 6-F | H | H | |
| 4.083 | 4-OCH₃ | 3-CH₂—CN, 4-Cl, 6-F | H | H | |
| 4.084 | 2-NH₂, 4-OCH₃ | 3-CH(CH₃)CN, 4-F | H | H | |
| 4.085 | 2-SCH₃ | 3-CH(CH₃)CN, 4-F | H | H | |
| 4.086 | 2-CF₃, 4-N(CH₃)₂ | 3-CH(CH₃)CN, 4-F | H | H | |
| 4.087 | 2-CH₃, 4-SCH₃ | 3-CH(CH₃)CN, 4-F | H | H | |
| 4.088 | 2-S(O)₂CH₃ | 3-CH(CH₃)CN, 4-F | H | H | |
| 4.089 | 2-NH₂, 4-OCH₃ | 3-CH₂—CN, 6-F | H | H | |
| 4.090 | 2-SCH₃ | 3-CH₂—CN, 6-F | H | H | |
| 4.091 | 2-CF₃, 4-N(CH₃)₂ | 3-CH₂—CN, 6-F | H | H | |
| 4.092 | 2-CH₃, 4-SCH₃ | 3-CH₂—CN, 6-F | H | H | |
| 4.093 | 2-S(O)₂CH₃ | 3-CH₂—CN, 6-F | H | H | |
| 4.094 | 2-NH₂, 4-OCH₃ | 3-CH₂—CN, 4-Cl, 6-F | H | H | |
| 4.095 | 2-SCH₃ | 3-CH₂—CN, 4-Cl, 6-F | H | H | |
| 4.096 | 2-CF₃, 4-N(CH₃)₂ | 3-CH₂—CN, 4-Cl, 6-F | H | H | |
| 4.097 | 2-CH₃, 4-SCH₃ | 3-CH₂—CN, 4-Cl, 6-F | H | H | |
| 4.098 | 2-S(O)₂CH₃ | 3-CH₂—CN, 4-Cl, 6-F | H | H | |
| 4.099 | 4-OCH₃ | 3-CHF₂, 4-F | H | H | |
| 4.100 | 4-OCH₃ | 3-CHF₂, 6-F | H | H | |
| 4.101 | 4-OCH₃ | 3-CHF₂, 4-F, 6-F | H | H | |
| 4.102 | 4-OCH₃ | 3-CHF₂, 4-Cl, 6-F | H | H | |
| 4.103 | 4-OCH₃ | 3-CHF₂ | H | H | |
| 4.104 | 2-Cl, 4-OCH₃ | 3-CHF₂, 4-F | H | H | |
| 4.105 | 2-Cl, 4-OCH₃ | 3-CHF₂, 6-F | H | H | |
| 4.106 | 2-01, 4-OCH₃ | 3-CHF₂, 4-F, 6-F | H | H | |
| 4.107 | 2-Cl, 4-OCH₃ | 3-CHF₂, 4-Cl, 6-F | H | H | |
| 4.108 | 2-Cl, 4-OCH₃ | 3-CHF₂ | H | H | |
| 4.109 | 2-CH₃, 4-OCH₃ | 3-CHF₂, 4-F | H | H | |
| 4.110 | 2-CH₃, 4-OCH₃ | 3-CHF₂, 6-F | H | H | |
| 4.111 | 2-CH₃, 4-OCH₃ | 3-CHF₂, 4-F, 6-F | H | H | |
| 4.112 | 2-CH₃, 4-OCH₃ | 3-CHF₂, 4-Cl, 6-F | H | H | |
| 4.113 | 2-CH₃, 4-OCH₃ | 3-CHF₂ | H | H | |
| 4.114 | 2-CF₃, 4-OCH₃ | 3-CHF₂, 4-F | H | H | |
| 4.115 | 2-CF₃, 4-OCH₃ | 3-CHF₂, 6-F | H | H | |
| 4.116 | 2-CF₃, 4-OCH₃ | 3-CHF₂, 4-F, 6-F | H | H | |
| 4.117 | 2-CF₃, 4-OCH₃ | 3-CHF₂, 4-Cl, 6-F | H | H | |
| 4.118 | 2-CF₃, 4-OCH₃ | 3-CHF₂ | H | H | |
| 4.119 | 2-CH₃, 4-OCH₃ | 3-CHF₂, 4-F | H | H | |
| 4.120 | 4-OCH₃ | 3-CHF₂, 4-F | H | H | |
| 4.121 | 4-OCH₃ | 3-CHF₂, 6-F | H | H | |
| 4.122 | 4-OCH₃ | 3-CHF₂, 4-F, 6-F | H | H | |
| 4.123 | 4-OCH₃ | 3-CHF₂, 4-Cl, 6-F | H | H | |
| 4.124 | 4-OCH₃ | 3-CHF₂ | H | H | |

TABLE 5
Compounds of formula Ib₄1:

| | |
|---|---|
| | (Ib₄1) |
| 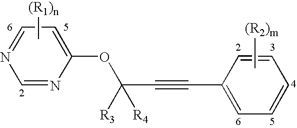 | |

| Comp. | | | | | Phys. data |
|---|---|---|---|---|---|
| no. | R₁ | R₂ | R₃ | R₄ | m.p. (° C.) |
| 5.001 | 2-F, 5-OCH₃ | 3-CH₂—CN, 4-F | H | H | |
| 5.002 | 2-Cl, 5-OCH₃ | 3-CH₂—CN, 4-F | H | H | |
| 5.003 | 5-OCH₃ | 3-CH₂—CN, 4-F | H | H | |
| 5.004 | 5-F | 3-CH₂—CN, 4-F | H | H | |
| 5.005 | 5-Cl | 3-CH₂—CN, 4-F | H | H | |
| 5.006 | 2-CH₃, 5-Cl | 3-CH₂—CN, 4-F | H | H | |
| 5.007 | 2-F, 5-Cl | 3-CH₂—CN, 4-F | H | H | |
| 5.008 | 5-CH₃ | 3-CH₂—CN, 4-F | H | H | |
| 5.009 | 2-F, 5-OCH₃ | 3-Cl | H | H | |
| 5.010 | 2-Cl, 5-OCH₃ | 3-Cl | H | H | |
| 5.011 | 5-OCH₃ | 3-Cl | H | H | |
| 5.012 | 5-F | 3-Cl | H | H | |
| 5.013 | 5-Cl | 3-Cl | H | H | |
| 5.014 | 2-CH₃, 5-Cl | 3-Cl | H | H | |
| 5.015 | 2-Cl, 5-F | 3-Cl | H | H | 99-100 |
| 5.016 | 5-CH₃ | 3-Cl | H | H | |
| 5.017 | 2-F, 5-OCH₃ | 3-CH₂—CN | H | H | |
| 5.018 | 2-Cl, 5-OCH₃ | 3-CH₂—CN | H | H | |
| 5.019 | 5-OCH₃ | 3-CH₂—CN | H | H | |
| 5.020 | 5-F | 3-CH₂—CN | H | H | |
| 5.021 | 5-Cl | 3-CH₂—CN | H | H | |
| 5.022 | 2-CH₃, 5-Cl | 3-CH₂—CN | H | H | crystalline |
| 5.023 | 2-Cl, 5-F | 3-CH₂—CN | H | H | |
| 5.024 | 5-CH₃ | 3-CH₂—CN | H | H | |
| 5.025 | 5-OCH₃ | 3-CH₂—CN | H | H | 96-97 |
| 5.026 | 2-CH₃, 5-OCH₃ | 3-CH₂—CN | H | H | 109-110 |
| 5.027 | 5-OCH₃ | 3-CH₂CN, 4-Cl | H | H | |
| 5.028 | 5-OCH₃ | 3-CH₂CN, 4-CH₃ | H | H | |
| 5.029 | 5-OCH₃ | 3-CH₂CN, 4-F, 6-F | H | H | |
| 5.030 | 5-OCH₃ | 3-CH₂CN, 4-Cl, 6-F | H | H | |
| 5.031 | 5-OCH₃ | 3-CH₂CN, 4-CH₃, | H | H | |
| | | 6-F | | | |
| 5.032 | 5-OCH₃ | 3-CHF₂ | H | H | |
| 5.033 | 5-OCH₃ | 3-CHF₂, 4-F | H | H | |
| 5.034 | 5-OCH₃ | 3-CHF₂, 4-Cl | H | H | |
| 5.035 | 5-OCH₃ | 3-CHF₂, 4-CH₃ | H | H | |
| 5.036 | 5-OCH₃ | 3-CHF₂, 4-F, 6-F | H | H | |
| 5.037 | 5-OCH₃ | 3-CHF₂, 4-Cl, 6-F | H | H | |
| 5.038 | 5-OCH₃ | 3-CHF₂, 4-CH₃, 6-F | H | H | |
| 5.039 | 5-OCH₃ | 3-CH(CH)₃CN | H | H | |
| 5.040 | 5-OCH₃ | 3-CH(CH)₃CN, 4-F | H | H | |
| 5.041 | 5-OCH₃ | 3-CH(CH)₃CN, 4-Cl | H | H | |
| 5.042 | 5-OCH₃ | 3-CH(CH)₃CN, | H | H | |
| | | 4-CH₃ | | | |
| 5.043 | 5-OCH₃ | 3-CH(CH)₃CN, 4-F, | H | H | |
| | | 6-F | | | |
| 5.044 | 5-OCH₃ | 3-CH(CH)₃CN, 4-Cl, | H | H | |
| | | 6-F | | | |
| 5.045 | 5-OCH₃ | 3-CH(CH)₃CN, | H | H | |
| | | 4-CH₃, 6-F | | | |
| 5.046 | 5-OCH₃, 2-CH₃ | 3-CH₂CN, 4-F | H | H | |
| 5.047 | 5-OCH₃, 2-CH₃ | 3-CH₂CN, 4-Cl | H | H | |
| 5.048 | 5-OCH₃, 2-CH₃ | 3-CH₂CN, 4-CH₃ | H | H | |
| 5.049 | 5-OCH₃, 2-CH₃ | 3-CH₂CN, 4-F, 6-F | H | H | |
| 5.050 | 5-OCH₃, 2-CH₃ | 3-CH₂CN, 4-Cl, 6-F | H | H | |
| 5.051 | 5-OCH₃, 2-CH₃ | 3-CH₂CN, 4-CH₃, | H | H | |
| | | 6-F | | | |
| 5.052 | 5-OCH₃, 2-F | 3-CH₂CN, 4-F | H | H | |
| 5.053 | 5-OCH₃, 2-F | 3-CH₂CN, 4-Cl | H | H | |
| 5.054 | 5-OCH₃, 2-F | 3-CH₂CN, 4-CH₃ | H | H | |
| 5.055 | 5-OCH₃, 2-F | 3-CH₂CN, 4-F, 6-F | H | H | |
| 5.056 | 5-OCH₃, 2-F | 3-CH₂CN, 4-Cl, 6-F | H | H | |
| 5.057 | 5-OCH₃, 2-F | 3-CH₂CN, 4-CH₃, | H | H | |
| | | 6-F | | | |
| 5.058 | 2-OCH₂CH₂CH₃, | 3-CH₂CN | H | H | resin |
| | 6-OCH₃ | | | | |
| 5.059 | 2-CH₃, 6-N-piperidyl | 3-CH₂CN | H | H | resin |
| 5.060 | 2-CF₃, 5-OCH₃ | 3-CH₂CN | H | H | crystalline |
| 5.061 | 2-SCH₃, 6-OCH₃ | 3-CH₂CN | H | H | amorphous |
| 5.062 | 2-CH₃, 6-Cl | 3-CH₂CN | H | H | amorphous |
| 5.063 | 2-SCH₃, 5-OCH₃ | 3-CH₂CN | H | H | 87-88 |
| 5.064 | 2-CH(CH₃)₂, | 3-CH₂CN | H | H | resin |
| | 6-CH₂OCH₂CH₃ | | | | |
| 5.065 | 5-NH₂, 6-Cl | 3-CH₂CN | H | H | oil |

TABLE 6
Compounds of formula Ib₅3:

| | |
|---|---|
| | (Ib₅3) |
| 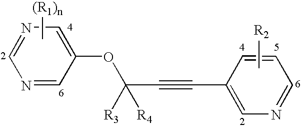 | |

| Comp. | | | | | Phys. data |
|---|---|---|---|---|---|
| no. | R₁ | R₂ | R₃ | R₄ | m.p. (° C.) |
| 6.001 | 2-CN; 4-OCH₃ | 5-CH₂—CN | H | H | |
| 6.002 | 2-Cl, 4-F | 5-CH₂—CN | H | H | |
| 6.003 | 2-Cl, 4-Cl | 5-CH₂—CN | H | H | |
| 6.004 | 2-F, 4-OCH₃ | 5-CH₂—CN | H | H | |
| 6.005 | 2-Cl, 4-OCH₃ | 5-CH₂—CN | H | H | |
| 6.006 | 2-Br, 4-OCH₃ | 5-CH₂—CN | H | H | |
| 6.007 | 2-CF₃, 4-Cl | 5-CH₂—CN | H | H | |
| 6.008 | 2-CF₃, 4-OCH₃ | 5-CH₂—CN | H | H | |
| 6.009 | 2-CH₃, 4-OCH₃ | 5-CH₂—CN | H | H | |
| 6.010 | 2-CH═NOCH₃, 4-OCH₃ | 5-CH₂—CN | H | H | |
| 6.011 | 2-CF₃, 4-F | 5-CH₂—CN | H | H | |
| 6.012 | 4-OCH₃ | 5-CH₂—CN | H | H | |
| 6.013 | 2-CN; 4-OCH₃ | 6-Cl | H | H | |
| 6.014 | 2-Cl, 4-F | 6-Cl | H | H | |
| 6.015 | 2-Cl, 4-Cl | 6-Cl | H | H | |
| 6.016 | 2-F, 4-OCH₃ | 6-Cl | H | H | |
| 6.017 | 2-Cl, 4-OCH₃ | 6-Cl | H | H | |
| 6.018 | 2-Br, 4-OCH₃ | 6-Cl | H | H | |
| 6.019 | 2-CF₃, 4-Cl | 6-Cl | H | H | |
| 6.020 | 2-CF₃, 4-OCH₃ | 6-Cl | H | H | |
| 6.021 | 2-CH₃, 4-OCH₃ | 6-Cl | H | H | |
| 6.022 | 2-CH═NOCH₃, 4-OCH₃ | 6-Cl | H | H | |
| 6.023 | 2-CF₃, 4-F | 6-Cl | H | H | |
| 6.024 | 4-OCH₃ | 6-Cl | H | H | |
| 6.025 | 4-OCH₃ | 6-Cl | F | F | |
| 6.026 | 2-CN, 4-OCH₃ | 6-CH₃ | H | H | |
| 6.027 | 2-Cl, 4-F | 6-CH₃ | H | H | |
| 6.028 | 2-Cl, 4-Cl | 6-CH₃ | H | H | |
| 6.029 | 2-F, 4-OCH₃ | 6-CH₃ | H | H | |
| 6.030 | 2-Cl, 4-OCH₃ | 6-CH₃ | H | H | |
| 6.031 | 2-Br, 4-OCH₃ | 6-CH₃ | H | H | |
| 6.032 | 2-CF₃, 4-Cl | 6-CH₃ | H | H | |
| 6.033 | 2-CF₃, 4-OCH₃ | 6-CH₃ | H | H | |
| 6.034 | 2-CH₃, 4-OCH₃ | 6-CH₃ | H | H | |
| 6.035 | 2-CH═NOCH₃, 4-OCH₃ | 6-CH₃ | H | H | |
| 6.036 | 2-CF₃, 4-F | 6-CH₃ | H | H | |
| 6.037 | 4-OCH₃ | 6-CH₃ | H | H | |
| 6.038 | 4-OCH₃ | 6-CH₃ | F | F | |

TABLE 7
Compounds of formula Ib₂1:

| | |
|---|---|
| | (Ib₂1) |
| 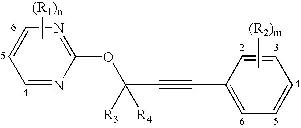 | |

| | | | | | Phys. |
|---|---|---|---|---|---|
| | | | | | data |
| Comp. | | | | | m.p. |
| no. | R₁ | R₂ | R₃ | R₄ | (° C.) |
| 7.001 | 5-Br | 3-Cl | H | H | |
| 7.002 | 5-Cl | 3-Cl | H | H | |
| 7.003 | 5-F | 3-Cl | H | H | |
| 7.004 | 4-CH₃, 6-CH₃ | 3-Cl | H | H | |
| 7.005 | 4-CF₃, 6-CF₃ | 3-Cl | H | H | |
| 7.006 | 5-Br | 3-CH₂—CN | H | H | solid |
| 7.007 | 5-Cl | 3-CH₂—CN | H | H | |
| 7.008 | 5-F | 3-CH₂—CN | H | H | 95-96 |
| 7.009 | 4-CH₃, 6-CH₃ | 3-CH₂—CN | H | H | |
| 7.010 | 4-CF₃, 6-CF₃ | 3-CH₂—CN | H | H | |
| 7.011 | 5-Br | 3-CH₂—CN | F | F | |
| 7.012 | 5-Br | 3-CH₂—CN, 4-F | H | H | |
| 7.013 | 5-Br | 3-CH(CH₃)CN | H | H | |
| 7.014 | H | 3-CH₂—CN | H | H | 98 |
| 7.015 | H | 3-Cl | H | H | oil |
| 7.016 | 5-Br | 3-Cl | H | H | 132- |
| | | | | | 133 |
| 7.017 | 5-Br | 3-Br | H | H | 142- |
| | | | | | 143 |
| 7.018 | H | 3,4-OCF₂O— | H | H | 94-95 |
| 7.019 | 4-CF₃ | 3-CH₂CN | H | H | resin |
| 7.020 | 5-Cl | 3-CH₂—CN, 4-Cl, 6-F | H | H | 152- |
| | | | | | 154 |
| 7.021 | 5-Cl | 3-CH₂—CN | H | H | 116- |
| | | | | | 118 |
| 7.022 | 5-Cl | 3-CH₂—CN, 4-F | H | H | solid |
| 7.023 | 5-F | 3-CH₂—CN, 6-F | H | H | 96-97 |
| 7.024 | 5-CH₂CH₂CH₃ | 3-CH₂—CN | H | H | resin |
| 7.025 | 4-OC₂H₅, 6-OC₂H₅ | 3-CH₂—CN | H | H | oil |
| 7.026 | 5-F | 3-CH₂—CN, 4-Cl, 6-F | H | H | 163- |
| | | | | | 164 |
| 7.027 | 5-F | 3-CH₂—CN, 4-F | H | H | solid |
| 7.028 | 5-Cl | 3-CH₂—CN, 6-F | H | H | 108- |
| | | | | | 110 |
| 7.029 | 5-Br | 3-OCH₂COOCH₃ | H | H | 95-97 |
| 7.030 | 5-Cl | 3-OCH₂COOCH₃ | H | H | 90-91 |
| 7.031 | 5-Cl | 3-CH₂—CN, 4-F, 6-F | H | H | oil |
| 7.032 | 5-Cl | 3-CH₂—CN, 4-NO₂ | H | H | 106- |
| | | | | | 109 |
| 7.033 | 5-CF₃ | 3-CH₂CN | H | H | |
| 7.034 | 5-CF₃ | 3-CH(CH₃)CN | H | H | |
| 7.035 | 5-CF₃ | 3-Cl | H | H | |
| 7.036 | 5-CF₃ | 3-CH₂—CN, 4-F | H | H | |
| 7.037 | 5-CF₃ | 3-CH(CH₃)CN, 4-F | H | H | |
| 7.038 | 5-CF₃ | 3-CH₂—CN, 6-F | H | H | |
| 7.039 | 5-CF₃ | 3-CH(CH₃)CN, 6-F | H | H | |
| 7.040 | 5-CF₃ | 3-CH₂—CN, 4-F, 6-F | H | H | |
| 7.041 | 5-CF₃ | 3-CH(CH₃)CN, 4-F, | H | H | |
| | | 6-F | | | |
| 7.042 | 5-CF₃ | 3-CH₂—CN, 4-Cl, 5-F | H | H | |
| 7.043 | 5-CF₃ | 3-CH(CH₃)CN, 4-Cl, | H | H | |
| | | 5-F | | | |
| 7.044 | 5-CF₃ | 3-Cl, 6-F | H | H | |
| 7.045 | 5-CF₃ | 3-CH₂—CN, 4-CN | H | H | |
| 7.046 | 5-CF₃ | 3-CH₂—CN, 4-CH₃ | H | H | |
| 7.047 | 5-CF₃ | 3-CH(CH₃)CN, | H | H | |
| | | 4-CH₃ | | | |
| 7.048 | 5-CF₃ | 3-Br | H | H | |
| 7.049 | 5-CF₃ | 3-I | H | H | |
| 7.050 | 5-CF₃ | 3-I, 6-F | H | H | |
| 7.051 | 5-CF₃ | 3-CH₂—CN, 4-NO₂ | H | H | |
| 7.052 | 5-CF₃ | 3-CH₂—CN, 4-NH₂ | H | H | |
| 7.053 | 5-CF₃ | 3-CHF₂ | H | H | |
| 7.054 | 5-CF₃ | 3-CHF₂, 4-F | H | H | |
| 7.055 | 5-CF₃ | 3-CHF₂, 6-F | H | H | |
| 7.056 | 5-CF₃ | 3-CHF₂, 4-F, 6-F | H | H | |
| 7.057 | 5-Br | 3-CH₂CN | H | H | |
| 7.058 | 5-Br | 3-CH(CH₃)CN | H | H | |
| 7.059 | 5-Br | 3-Cl | H | H | 132- |
| | | | | | 133 |
| 7.060 | 5-Br | 3-CH₂—CN, 4-F | H | H | |
| 7.061 | 5-Br | 3-CH(CH₃)CN, 4-F | H | H | |
| 7.062 | 5-Br | 3-CH₂—CN, 6-F | H | H | |
| 7.063 | 5-Br | 3-CH(CH₃)CN, 6-F | H | H | |
| 7.064 | 5-Br | 3-CH₂—CN, 4-F, 6-F | H | H | |
| 7.065 | 5-Br | 3-CH(CH₃)CN, 4-F, | H | H | |
| | | 6-F | | | |
| 7.066 | 5-Br | 3-CH₂—CN, 4-Cl, 5-F | H | H | |
| 7.067 | 5-Br | 3-CH(CH₃)CN, 4-Cl, | H | H | |
| | 5-F | | | | |
| 7.068 | 5-Br | 3-Cl, 6-F | H | H | |
| 7.069 | 5-Br | 3-CH₂—CN, 4-CN | H | H | |
| 7.070 | 5-Br | 3-CH₂—CN, 4-CH₃ | H | H | |
| 7.071 | 5-Br | 3-CH(CH₃)CN, | H | H | |
| | | 4-CH₃ | | | |
| 7.072 | 5-Br | 3-Br | H | H | |
| 7.073 | 5-Br | 3-I | H | H | |
| 7.074 | 5-Br | 3-I, 6-F | H | H | |
| 7.075 | 5-Br | 3-CH₂—CN, 4-NO₂ | H | H | |
| 7.076 | 5-Br | 3-CH₂-CN, 4-NH₂ | H | H | |
| 7.077 | 5-Br | 3-CHF₂ | H | H | |
| 7.078 | 5-Br | 3-CHF₂, 4-F | H | H | |
| 7.079 | 5-Br | 3-CHF₂, 6-F | H | H | |
| 7.080 | 5-Br | 3-CHF₂, 4-F, 6-F | H | H | |
| 7.081 | 5-Cl | 3-CH₂CN | H | H | |
| 7.082 | 5-Cl | 3-CH(CH₃)CN | H | H | |
| 7.083 | 5-Cl | 3-Cl | H | H | |
| 7.084 | 5-Cl | 3-CH₂—CN, 4-F | H | H | |
| 7.085 | 5-Cl | 3-CH(CH₃)CN, 4-F | H | H | |
| 7.086 | 5-Cl | 3-CH₂—CN, 6-F | H | H | |
| 7.087 | 5-Cl | 3-CH(CH₃)CN, 6-F | H | H | |
| 7.088 | 5-Cl | 3-CH₂—CN, 4-F, 6-F | H | H | |
| 7.089 | 5-Cl | 3-CH(CH₃)CN, 4-F, | H | H | |
| | | 6-F | | | |
| 7.090 | 5-Cl | 3-CH₂—CN, 4-Cl, 5-F | H | H | |
| 7.091 | 5-Cl | 3-CH(CH₃)CN, 4-Cl, | H | H | |
| | | 5-F | | | |
| 7.092 | 5-Cl | 3-Cl, 6-F | H | H | |
| 7.093 | 5-Cl | 3-CH₂—CN, 4-ON | H | H | |
| 7.094 | 5-Cl | 3-CH₂—CN, 4-CH₃ | H | H | |
| 7.095 | 5-Cl | 3-OH(CH₃)CN, | H | H | |
| | | 4-CH₃ | | | |
| 7.096 | 5-Cl | 3-Br | H | H | |
| 7.097 | 5-Cl | 3-I | H | H | |
| 7.098 | 5-Cl | 3-I, 6-F | H | H | |
| 7.099 | 5-Cl | 3-CH₂—CN, 4-NO₂ | H | H | |
| 7.100 | 5-Cl | 3-CH₂—CN, 4-NH₂ | H | H | |
| 7.101 | 5-Cl | 3-CHF₂ | H | H | |
| 7.102 | 5-Cl | 3-CHF₂, 4-F | H | H | |
| 7.103 | 5-Cl | 3-CHF₂, 6-F | H | H | |
| 7.104 | 5-Cl | 3-CHF₂, 4-F, 6-F | H | H | |
| 7.105 | 5-F | 3-CH₂CN | H | H | |
| 7.106 | 5-F | 3-CH(CH₃)CN | H | H | |
| 7.107 | 5-F | 3-Cl | H | H | |
| 7.108 | 5-F | 3-CH₂—CN, 4-F | H | H | |
| 7.109 | 5-F | 3-CH(CH₃)CN, 4-F | H | H | |
| 7.110 | 5-F | 3-CH₂—CN, 6-F | H | H | |
| 7.111 | 5-F | 3-CH(CH₃)CN, 6-F | H | H | |
| 7.112 | 5-F | 3-CH₂—CN, 4-F, 6-F | H | H | |
| 7.113 | 5-F | 3-CH(CH₃)CN, 4-F, | H | H | |
| | | 6-F | | | |
| 7.114 | 5-F | 3-CH₂—CN, 4-Cl, 6-F | H | H | |
| 7.115 | 5-F | 3-CH(CH₃)CN, 4-Cl, | H | H | |
| | | 6-F | | | |
| 7.116 | 5-F | 3-Cl, 6-F | H | H | |
| 7.117 | 5-F | 3-CH₂—CN, 4-CN | H | H | |
| 7.118 | 5-F | 3-CH₂—CN, 4-CH₃ | H | H | |
| 7.119 | 5-F | 3-CH(CH₃)CN, | H | H | |
| | | 4-CH₃ | | | |
| 7.120 | 5-F | 3-Br | H | H | |
| 7.121 | 5-F | 3-I | H | H | |
| 7.122 | 5-F | 3-I, 6-F | H | H | |
| 7.123 | 5-F | 3-CH₂—CN, 4-NO₂ | H | H | |
| 7.124 | 5-F | 3-CH₂—CN, 4-NH₂ | H | H | |
| 7.125 | 5-F | 3-CHF₂ | H | H | |
| 7.126 | 5-F | 3-CHF₂, 4-F | H | H | |
| 7.127 | 5-F | 3-CHF₂, 6-F | H | H | |
| 7.128 | 5-F | 3-CHF₂, 4-F, 6-F | H | H | |
| 7.129 | 5-CN | 3-CH₂CN | H | H | |
| 7.130 | 5-CN | 3-CH(CH₃)CN | H | H | |
| 7.131 | 5-CN | 3-Cl | H | H | |
| 7.132 | 5-CN | 3-CH₂—CN, 4-F | H | H | |
| 7.133 | 5-CN | 3-CH(CH₃)CN, 4-F | H | H | |
| 7.134 | 5-CN | 3-CH₂—CN, 6-F | H | H | |
| 7.135 | 5-CN | 3-CH(CH₃)CN, 6-F | H | H | |
| 7.136 | 5-CN | 3-CH₂—CN, 4-F, 6-F | H | H | |
| 7.137 | 5-CN | 3-CH(CH₃)CN, 4-F, | H | H | |
| | | 6-F | | | |
| 7.138 | 5-CN | 3-CH₂—CN, 4-Cl, 6-F | H | H | |
| 7.139 | 5-CN | 3-CH(CH₃)CN, 4-Cl, | H | H | |
| | | 6-F | | | |
| 7.140 | 5-CN | 3-Cl, 6-F | H | H | |
| 7.141 | 5-CN | 3-CH₂—CN, 4-CN | H | H | |
| 7.142 | 5-CN | 3-CH₂—CN, 4-CH₃ | H | H | |
| 7.143 | 5-CN | 3-CH(CH₃)CN, | H | H | |
| | | 4-CH₃ | | | |
| 7.144 | 5-CN | 3-Br | H | H | |
| 7.145 | 5-CN | 3-I | H | H | |
| 7.146 | 5-CN | 3-I, 6-F | H | H | |
| 7.147 | 5-CN | 3-CH₂—CN, 4-NO₂ | H | H | |
| 7.148 | 5-CN | 3-CH₂-CN, 4-NH₂ | H | H | |
| 7.149 | 5-CN | 3-CHF₂ | H | H | |
| 7.150 | 5-CN | 3-CHF₂, 4-F | H | H | |
| 7.151 | 5-CN | 3-CHF₂, 6-F | H | H | |
| 7.152 | 5-CN | 3-CHF₂, 4-F, 6-F | H | H | |
| 7.153 | 5-CH₃ | 3-CH₂CN | H | H | |
| 7.154 | 5-CH₃ | 3-CH(CH₃)CN | H | H | |
| 7.155 | 5-CH₃ | 3-Cl | H | H | |
| 7.156 | 5-CH₃ | 3-CH₂—CN, 4-F | H | H | |
| 7.157 | 5-CH₃ | 3-CH(CH₃)CN, 4-F | H | H | |
| 7.158 | 5-CH₃ | 3-CH₂—CN, 6-F | H | H | |
| 7.159 | 5-CH₃ | 3-CH(CH₃)CN, 6-F | H | H | |
| 7.160 | 5-CH₃ | 3-CH₂—CN, 4-F, 6-F | H | H | |
| 7.161 | 5-CH₃ | 3-CH(CH₃)CN, 4-F, | H | H | |
| | | 6-F | | | |
| 7.162 | 5-CH₃ | 3-CH₂-CN, 4-Cl, 5-F | H | H | |
| 7.163 | 5-CH₃ | 3-CH(CH₃)CN, 4-Cl, | H | H | |
| | | 5-F | | | |
| 7.164 | 5-CH₃ | 3-Cl, 6-F | H | H | |
| 7.165 | 5-CH₃ | 3-CH₂—CN, 4-CN | H | H | |
| 7.166 | 5-CH₃ | 3-CH₂—CN, 4-CH₃ | H | H | |
| 7.167 | 5-CH₃ | 3-CH(CH₃)CN, | H | H | |
| | | 4-CH₃ | | | |
| 7.168 | 5-CH₃ | 3-Br | H | H | |
| 7.169 | 5-CH₃ | 3-I | H | H | |
| 7.170 | 5-CH₃ | 3-I, 6-F | H | H | |
| 7.171 | 5-CH₃ | 3-CH₂—CN, 4-NO₂ | H | H | |
| 7.172 | 5-CH₃ | 3-CH₂—CN, 4-NH₂ | H | H | |
| 7.173 | 5-CH₃ | 3-CHF₂ | H | H | |
| 7.174 | 5-CH₃ | 3-CHF₂, 4-F | H | H | |
| 7.175 | 5-CH₃ | 3-CHF₂, 6-F | H | H | |
| 7.176 | 5-CH₃ | 3-CHF₂, 4-F, 6-F | H | H | |
| 7.177 | 5-CH₂CH₃ | 3-CH₂CN | H | H | |
| 7.178 | 5-CH₂CH₃ | 3-CH(CH₃)CN | H | H | |
| 7.179 | 5-CH₂CH₃ | 3-Cl | H | H | |
| 7.180 | 5-CH₂CH₃ | 3-CH₂—CN, 4-F | H | H | |
| 7.181 | 5-CH₂CH₃ | 3-CH(CH₃)CN, 4-F | H | H | |
| 7.182 | 5-CH₂CH₃ | 3-CH₂—CN, 6-F | H | H | |
| 7.183 | 5-CH₂CH₃ | 3-CH(CH₃)CN, 6-F | H | H | |
| 7.184 | 5-CH₂CH₃ | 3-CH₂—CN, 4-F, 6-F | H | H | |
| 7.185 | 5-CH₂CH₃ | 3-CH(CH₃)CN, 4-F, | H | H | |
| | | 6-F | | | |
| 7.186 | 5-CH₂CH₃ | 3-CH₂—CN, 4-Cl, 6-F | H | H | |
| 7.187 | 5-CH₂CH₃ | 3-CH(CH₃)CN, 4-Cl, | H | H | |
| | | 6-F | | | |
| 7.188 | 5-CH₂CH₃ | 3-Cl, 6-F | H | H | |
| 7.189 | 5-CH₂CH₃ | 3-CH₂—CN, 4-CN | H | H | |
| 7.190 | 5-CH₂CH₃ | 3-CH₂—CN, 4-CH₃ | H | H | |
| 7.191 | 5-CH₂CH₃ | 3-CH(CH₃)CN, | H | H | |
| | | 4-CH₃ | | | |
| 7.192 | 5-CH₂CH₃ | 3-Br | H | H | |
| 7.193 | 5-CH₂CH₃ | 3-I | H | H | |
| 7.194 | 5-CH₂CH₃ | 3-I, 6-F | H | H | |
| 7.195 | 5-CH₂CH₃ | 3-CH₂—CN, 4-NO₂ | H | H | |
| 7.196 | 5-CH₂CH₃ | 3-CH₂—CN, 4-NH₂ | H | H | |
| 7.197 | 5-CH₂CH₃ | 3-CHF₂ | H | H | |
| 7.198 | 5-CH₂CH₃ | 3-CHF₂, 4-F | H | H | |
| 7.199 | 5-CH₂CH₃ | 3-CHF₂, 6-F | H | H | |
| 7.200 | 5-CH₂CH₃ | 3-CHF₂, 4-F, 6-F | H | H | |
| 7.201 | 5-CH(CH₃)₂ | 3-CH₂CN | H | H | |
| 7.202 | 5-CH(CH₃)₂ | 3-CH(CH₃)CN | H | H | |
| 7.203 | 5-CH(CH₃)₂ | 3-Cl | H | H | |
| 7.204 | 5-CH(CH₃)₂ | 3-CH₂—CN, 4-F | H | H | |
| 7.205 | 5-CH(CH₃)₂ | 3-CH(CH₃)CN, 4-F | H | H | |
| 7.206 | 5-CH(CH₃)₂ | 3-CH₂—CN, 6-F | H | H | |
| 7.207 | 5-CH(CH₃)₂ | 3-CH(CH₃)CN, 6-F | H | H | |
| 7.208 | 5-CH(CH₃)₂ | 3-CH₂—CN, 4-F, 6-F | H | H | |
| 7.209 | 5-CH(CH₃)₂ | 3-CH(CH₃)CN, 4-F, | H | H | |
| | | 6-F | | | |
| 7.210 | 5-CH(CH₃)₂ | 3-CH₂—CN, 4-Cl, 5-F | H | H | |
| 7.211 | 5-CH(CH₃)₂ | 3-CH(CH₃)CN, 4-Cl, | H | H | |
| | | 5-F | | | |
| 7.212 | 5-CH(CH₃)₂ | 3-Cl, 6-F | H | H | |
| 7.213 | 5-CH(CH₃)₂ | 3-CH₂—CN, 4-CN | H | H | |
| 7.214 | 5-CH(CH₃)₂ | 3-CH₂-CN, 4-CH₃ | H | H | |
| 7.215 | 5-CH(CH₃)₂ | 3-CH(CH₃)CN, | H | H | |
| | | 4-CH₃ | | | |
| 7.216 | 5-CH(CH₃)₂ | 3-Br | H | H | |
| 7.217 | 5-CH(CH₃)₂ | 3-I | H | H | |
| 7.218 | 5-CH(CH₃)₂ | 3-I, 6-F | H | H | |
| 7.219 | 5-CH(CH₃)₂ | 3-CH₂—CN, 4-NO₂ | H | H | |
| 7.220 | 5-CH(CH₃)₂ | 3-CH₂—CN, 4-NH₂ | H | H | |
| 7.221 | 5-CH(CH₃)₂ | 3-CHF₂ | H | H | |
| 7.222 | 5-CH(CH₃)₂ | 3-CHF₂, 4-F | H | H | |
| 7.223 | 5-CH(CH₃)₂ | 3-CHF₂, 6-F | H | H | |
| 7.224 | 5-CH(CH₃)₂ | 3-CHF₂, 4-F, 6-F | H | H | |
| 7.225 | 5-CH₂CH₂CH₃ | 3-CH₂CN | H | H | |
| 7.226 | 5-CH₂CH₂CH₃ | 3-CH(CH₃)CN | H | H | |
| 7.227 | 5-CH₂CH₂CH₃ | 3-Cl | H | H | |
| 7.228 | 5-CH₂CH₂CH₃ | 3-CH₂—CN, 4-F | H | H | |
| 7.229 | 5-CH₂CH₂CH₃ | 3-CH(CH₃)CN, 4-F | H | H | |
| 7.230 | 5-CH₂CH₂CH₃ | 3-CH₂—CN, 6-F | H | H | |
| 7.231 | 5-CH₂CH₂CH₃ | 3-CH(CH₃)CN, 6-F | H | H | |
| 7.232 | 5-CH₂CH₂CH₃ | 3-CH₂—CN, 4-F, 6-F | H | H | |
| 7.233 | 5-CH₂CH₂CH₃ | 3-CH(CH₃)CN, 4-F, | H | H | |
| | | 6-F | | | |
| 7.234 | 5-CH₂CH₂CH₃ | 3-CH₂—CN, 4-Cl, 6-F | H | H | |
| 7.235 | 5-CH₂CH₂CH₃ | 3-CH(CH₃)CN, 4-Cl, | H | H | |
| | | 6-F | | | |
| 7.236 | 5-CH₂CH₂CH₃ | 3-Cl, 6-F | H | H | |
| 7.237 | 5-CH₂CH₂CH₃ | 3-CH₂—CN, 4-CN | H | H | |
| 7.238 | 5-CH₂CH₂CH₃ | 3-CH₂-CN, 4-CH₃ | H | H | |
| 7.239 | 5-CH₂CH₂CH₃ | 3-CH(CH₃)CN, | H | H | |
| | | 4-CH₃ | | | |
| 7.240 | 5-CH₂CH₂CH₃ | 3-Br | H | H | |
| 7.241 | 5-CH₂CH₂CH₃ | 3-I | H | H | |
| 7.242 | 5-CH₂CH₂CH₃ | 3-I, 6-F | H | H | |
| 7.243 | 5-CH₂CH₂CH₃ | 3-CH₂-CN, 4-NO₂ | H | H | |
| 7.244 | 5-CH₂CH₂CH₃ | 3-CH₂-CN, 4-NH₂ | H | H | |
| 7.245 | 5-CH₂CH₂CH₃ | 3-CHF₂ | H | H | |
| 7.246 | 5-CH₂CH₂CH₃ | 3-CHF₂, 4-F | H | H | |
| 7.247 | 5-CH₂CH₂CH₃ | 3-CHF₂, 6-F | H | H | |
| 7.248 | 5-CH₂CH₂CH₃ | 3-CHF₂, 4-F, 6-F | H | H | |
| 7.249 | 5-CH═NOCH₃ | 3-CH₂CN | H | H | |
| 7.250 | 5-CH═NOCH₃ | 3-CH(CH₃)CN | H | H | |
| 7.251 | 5-CH═NOCH₃ | 3-Cl | H | H | |
| 7.252 | 5-CH═NOCH₃ | 3-CH₂—CN, 4-F | H | H | |
| 7.253 | 5-CH═NOCH₃ | 3-CH(CH₃)CN, 4-F | H | H | |
| 7.254 | 5-CH═NOCH₃ | 3-CH₂—CN, 6-F | H | H | |
| 7.255 | 5-CH═NOCH₃ | 3-CH(CH₃)CN, 6-F | H | H | |
| 7.256 | 5-CH═NOCH₃ | 3-CH₂—CN, 4-F) 6-F | H | H | |
| 7.257 | 5-CH═NOCH₃ | 3-CH(CH₃)CN, 4-F) | H | H | |
| | | 6-F | | | |
| 7.258 | 5-CH═NOCH₃ | 3-CH₂—CN, 4-Cl, 6-F | H | H | |
| 7.259 | 5-CH═NOCH₃ | 3-CH(CH₃)CN, 4-Cl, | H | H | |
| | | 6-F | | | |
| 7.260 | 5-CH═NOCH₃ | 3-Cl, 6-F | H | H | |
| 7.261 | 5-CH═NOCH₃ | 3-CH₂—CN, 4-CN | H | H | |
| 7.262 | 5-CH═NOCH₃ | 3-CH₂—CN, 4-CH₃ | H | H | |
| 7.263 | 5-CH═NOCH₃ | 3-CH(CH₃)CN, | H | H | |
| | | 4-CH₃ | | | |
| 7.264 | 5-CH═NOCH₃ | 3-Br | H | H | |
| 7.265 | 5-CH═NOCH₃ | 3-I | H | H | |
| 7.266 | 5-CH═NOCH₃ | 3-I, 6-F | H | H | |
| 7.267 | 5-CH═NOCH₃ | 3-CH₂—CN, 4-NO₂ | H | H | |
| 7.268 | 5-CH═NOCH₃ | 3-CH₂-CN, 4-NH₂ | H | H | |
| 7.269 | 5-CH═NOCH₃ | 3-CHF₂ | H | H | |
| 7.270 | 5-CH═NOCH₃ | 3-CHF₂, 4-F | H | H | |
| 7.271 | 5-CH═NOCH₃ | 3-CHF₂, 6-F | H | H | |
| 7.272 | 5-CH═NOCH₃ | 3-CHF₂, 4-F, 6-F | H | H | |
| 7.273 | H (n = 0) | 3-CHF₂, 4-F, 6-F | H | H | |
| 7.274 | H (n = 0) | 3-CHF₂, 4-Cl, 6-F | H | H | |
| 7.275 | H (n = 0) | 3-CHF₂, 4-F | H | H | |
| 7.276 | H (n = 0) | 3-CHF₂, 6-F | H | H | |
| 7.277 | H (n = 0) | 3-CHF₂ | H | H | |
| 7.278 | H (n = 0) | 3-CH₂—CN, 4-F, 6-F | H | H | |
| 7.279 | H (n = 0) | 3-CH₂—CN, 4-Cl, 6-F | H | H | |
| 7.280 | H (n = 0) | 3-CH₂—CN, 4-F | H | H | |
| 7.281 | H (n = 0) | 3-CH₂—CN, 6-F | H | H | |
| 7.282 | H (n = 0) | 3-CH₂—CN | H | H | |
| 7.283 | H (n = 0) | 3-CH(CH₃)CN, | H | H | |
| | | 4-F, 6-F | | | |
| 7.284 | H (n = 0) | 3-CH(CH₃)CN, | H | H | |
| | | 4-F | | | |
| 7.285 | H (n = 0) | 3-CH(CH₃)CN, | H | H | |
| | | 6-F | | | |
| 7.286 | H (n = 0) | 3-CH(CH₃)CN | H | H | |
| 7.287 | H (n = 0) | 3-Cl | H | H | |
| 7.288 | H (n = 0) | 3-Br | H | H | |
| 7.289 | H (n = 0) | 3-F | H | H | |
| 7.290 | H (n = 0) | 3-CH₃ | H | H | |
| 7.291 | H (n = 0) | 3-I | H | H | |

TABLE 8
Compounds of formula Ie₆1:

| | |
|---|---|
| | (Ie₆1) |
| 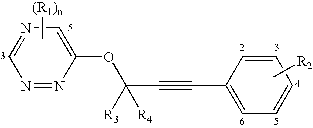 | |

| | | | | | Phys. |
|---|---|---|---|---|---|
| | | | | | data |
| Comp. | | | | | m.p. |
| no. | R₁ | R₂ | R₃ | R₄ | (° C.) |
| 8.001 | 3-CN, 5-OCH₃ | 3-CH₂—CN | H | H | |
| 8.002 | 3-Cl, 5-F | 3-CH₂—CN | H | H | |
| 8.003 | 3-Cl, 5-Cl | 3-CH₂—CN | H | H | |
| 8.004 | 3-F, 5-OCH₃ | 3-CH₂—CN | H | H | |
| 8.005 | 3-Cl, 5-OCH₃ | 3-CH₂—CN | H | H | |
| 8.006 | 3-Br, 5-OCH₃ | 3-CH₂—CN | H | H | |
| 8.007 | 3-CF₃, 5-Cl | 3-CH₂—CN | H | H | |
| 8.008 | 3-CF₃, 5-OCH₃ | 3-CH₂—CN | H | H | |
| 8.009 | 3-CH₃, 5-OCH₃ | 3-CH₂—CN | H | H | |
| 8.010 | 3-CH═NOCH₃, 5-OCH₃ | 3-CH₂—CN | H | H | |
| 8.011 | 3-CF₃, 5-F | 3-CH₂—CN | H | H | |
| 8.012 | 5-OCH₃ | 3-CH₂—CN | H | H | |
| 8.013 | 5-OCH₃ | 3-CH₂—CN | F | F | |
| 8.014 | 5-OCH₃ | 3-CHF₂ | H | H | |
| 8.015 | 5-OCH₃ | 3-CHF₂, 4-F | H | H | |
| 8.016 | H (n = 0) | 3-CH₂—CN | H | H | |
| 8.017 | H (n = 0) | 3-CHF₂ | H | H | |
| 8.018 | H (n = 0) | 3-CHF₂, 4-F | H | H | |
| 8.019 | H (n = 0) | 3-CH₂—CN, 4-F | H | H | |

TABLE 9
Compounds of formula Ic₃1:

| | |
|---|---|
| | (Ic₃1) |
| 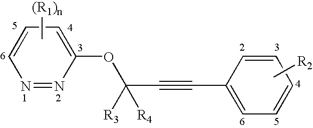 | |

| Comp. | | | | | Phys. data |
|---|---|---|---|---|---|
| no. | R₁ | R₂ | R₃ | R₄ | m.p. (° C.) |
| 9.001 | 6-Cl | 3-Cl | H | H | 83-84 |
| 9.002 | 6-Cl | 4-Cl | H | H | solid |
| 9.003 | 6-Cl | 3-CH₂—CN | H | H | 97-98 |
| 9.004 | 4-OCH₃ | 3-CH₂—CN | H | H | |
| 9.005 | 4-OCH₃ | 4-Cl | H | H | |
| 9.006 | 4-OCH₃ | 3-Cl | H | H | |
| 9.007 | 4-OCH₃ | 3-CH₂—CN, 4-F | H | H | |
| 9.008 | 4-OCH₃ | 3-CH₂—CN, 4-Cl | H | H | |
| 9.009 | 4-OCH₃ | 3-CH₂—CN, 4-CH₃ | H | H | |
| 9.010 | 4-OCH₃ | 3-CH₂—CN, 4-Cl, 6-F | H | H | |
| 9.011 | 4-OCH₃, 6-Cl | 3-CH₂—CN | H | H | |
| 9.012 | 4-OCH₃, 6-Cl | 4-Cl | H | H | |
| 9.013 | 4-OCH₃, 6-Cl | 3-Cl | H | H | |
| 9.014 | 4-OCH₃, 6-Cl | 3-CH₂—CN, 4-F | H | H | |
| 9.015 | 4-OCH₃, 6-Cl | 3-CH₂—CN, 4-Cl | H | H | |
| 9.016 | 4-OCH₃, 6-Cl | 3-CH₂—CN, 4-CH₃ | H | H | |
| 9.017 | 4-OCH₃, 6-Cl | 3-CH₂—CN, 4-Cl, 6-F | H | H | |
| 9.018 | 4-F | 3-CH₂—CN | H | H | |
| 9.019 | 4-F, 6-Cl | 3-CH₂—CN | H | H | |
| 9.020 | 4-F, 6-CF₃ | 3-CH₂—CN | H | H | |
| 9.021 | 4-F, 6-Br | 3-CH₂—CN | H | H | |
| 9.022 | 4-Cl, 6-Cl | 3-CH₂—CN | H | H | |
| 9.023 | 4-Cl | 3-CH₂—CN | H | H | |
| 9.024 | 6-F | 3-CH₂—CN | H | H | |
| 9.025 | 4-OCH₃, 6-CF₃ | 3-CH₂—CN, 4-F | H | H | |
| 9.026 | 4-OCH₃, 6-CF₃ | 3-CH₂—CN | H | H | |
| 9.027 | 4-OCH₃, 6-Cl | 3-CH₂—CN, 6-F | H | H | |
| 9.028 | 4-F, 6-Cl | 3-CH₂—CN, 6-F | H | H | |
| 9.029 | 4-F, 6-Cl | 3-CH₂—CN, 4-F | H | H | |
| 9.030 | 4-F, 6-Cl | 3-CHF₂, 6-F | H | H | |
| 9.031 | 4-OCH₃, 6-Cl | 3-CHF₂, 4-F | H | H | |
| 9.032 | 4-F, 6-Cl | 3-CHF₂ | H | H | |
| 9.033 | 4-OCH₃, 6-Cl | 3-CHF₂ | H | H | |
| 9.034 | 4-OCH₃ | 3-CHF₂ | H | H | |
| 9.035 | 4-OCH₃ | 3-CHF₂, 6-F | H | H | |
| 9.036 | 4-OCH₃ | 3-CHF₂, 4-F | H | H | |
| 9.037 | 4-OCH₃ | 3-CHF₂, 4-F, 6-F | H | H | |
| 9.038 | 4-OCH₃ | 3-CHF₂, 4-Cl, 6-F | H | H | |
| 9.039 | 4-OCH₃, 6-Cl | 3-CHF₂, 6-F | H | H | |
| 9.040 | 4-OCH₃, 6-Cl | 3-CHF₂, 4-F, 6-F | H | H | |
| 9.041 | 4-OCH₃, 6-Cl | 3-CHF₂, 4-Cl, 6-F | H | H | |

### BIOLOGICAL EXAMPLES

### Example B1

### Herbicidal Action Prior to Emergence of the Plants (Pre-Emergence Action)

Monocotyledonous and dicotyledonous test plants are sown in standard soil in pots. Immediately after sowing, the test compounds, in the form of an aqueous suspension (prepared from a wettable powder (Example F3, b) according to WO 97/34485) or in the form of an emulsion (prepared from an emulsifiable concentrate (Example F1, c) according to WO 97/34485), are applied by spraying, in an optimum concentration (500 litres of water per ha). The test plants are then grown in a greenhouse under optimum conditions.

After a test duration of 4 weeks, the test is evaluated in accordance with a scale of nine ratings (1=total damage, 9=no action). Ratings of from 1 to 4 (especially from 1 to 3) indicate good to very good herbicidal action.

Test plants: Panicum (Pani), Digitaria (Digit), Euphorbia (Euph), Sida, Abutilon (Abut), Amaranthus (Amar), Chenopodium (Cheno), Stellaria (Stell).

TABLE B1
Concentration: 1000 g of active compound per ha

| | Test plant: | | | | | | | |
|---|---|---|---|---|---|---|---|---|
| Comp. no. | Pani | Digit | Euph | Sida | Abut | Amar | Cheno | Stell |
| 1.041 | 1 | 1 | 1 | 1 | 1 | 1 | 1 | 1 |
| 1.048 | 4 | 2 | 1 | 1 | 1 | 1 | 1 | 1 |
| 7.006 | 2 | — | 1 | — | 2 | — | 1 | 1 |
| 7.022 | 1 | 1 | 3 | 1 | 1 | 1 | 3 | 1 |

The same results are obtained when the compounds of formula I are formulated analogously to the other Examples of WO 97/34485.

### Example B2

### Post-Emergence Herbicidal Action

Monocotyledonous and dicotyledonous test plants are sown in standard soil in pots. When the test plants are at the 2- to 3-leaf stage, the test compounds, in the form of an aqueous suspension (prepared from a wettable powder (Example F3, b) according to WO 97/34485) or in the form of an emulsion (prepared from an emulsifiable concentrate (Example F1, c) according to WO 97/34485), are applied by spraying, in an optimum concentration (500 litres of water per ha). The test plants are then grown on in a greenhouse under optimum conditions.

After a test duration of 2 to 3 weeks, the test is evaluated in accordance with a scale of nine ratings (1=total damage, 9=no action). Ratings of from 1 to 4 (especially from 1 to 3) indicate good to very good herbicidal action.

Test plants. Panicum (Pani), Euphorbia (Euph), Abutilon (Abut), Amaranthus (Amara), Stellaria (Stell), Veronica (Vero).

TABLE B2
Concentration: 1000 g of active compound per ha

Test plant:

| Comp. no. | Pani | Euph | Abut | Amar | Stell | Vero |
|---|---|---|---|---|---|---|
| 1.041 | 2 | 1 | 2 | 2 | 2 | 2 |
| 1.048 | 4 | 1 | 3 | 1 | 2 | 2 |
| 4.033 | 2 | 2 | 3 | 3 | 2 | 4 |
| 5.025 | 2 | 1 | 1 | 4 | 1 | 4 |
| 7.006 | 5 | 1 | 3 | 2 | 4 | 5 |
| 7.008 | 2 | 2 | 2 | 2 | 2 | 2 |
| 7.021 | 2 | 1 | 2 | 1 | 3 | 3 |
| 7.022 | 2 | 1 | — | 1 | 3 | 2 |
| 7.027 | 1 | 2 | 4 | 2 | 3 | 4 |
| 7.028 | — | 1 | — | 1 | 1 | 2 |
| 7.031 | 2 | 3 | 3 | 2 | 3 | 2 |

In the two Tables above, “-” means that there are no data available for the compounds and weeds in question.

The same results are obtained when the compounds of formula I are formulated analogously to the other Examples of WO 97/34485.

1. A compound of formula I 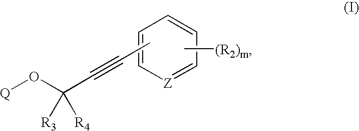
wherein
Q is a group 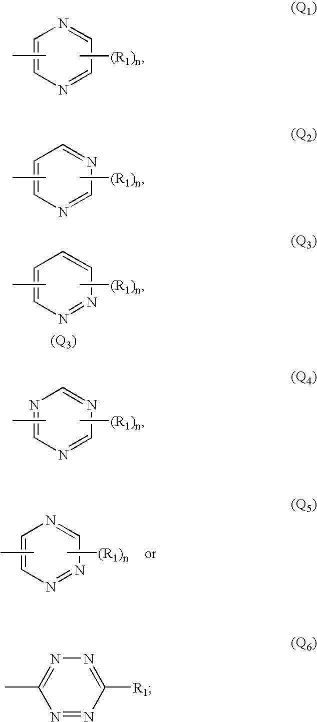
Z is ═N—, 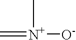
or ═C(R₂)—;
n is 0, 1, 2 or 3;
each R₁ independently is halogen, —CN, —SCN, —SF₅, —NO₂, —NR₅R₆, —CO₂R₇, —CONR₈R₉, —C(R₁₀)═NOR₁₁, —COR₁₂, —OR₁₃, —SR₁₄, —SOR₁₅, —SO₂R₁₆, —OSO₂R₁₇, C₁-C₈alkyl, C₂-C₈alkenyl, C₂-C₈alkynyl or C₃-C₆cycloalkyl; or is C₁-C₈alkyl, C₂-C₈alkenyl or C₂-C₈alkynyl mono- to penta-substituted by halogen or mono-, di- or tri-substituted by —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —OR₂₉, —SR₃₀, —SOR₃₁, —SO₂R₃₂ or by C₃-C₆cycloalkyl; or
each R₁ independently is C₃-C₆cycloalkyl mono- to penta-substituted by halogen or mono-, di- or tri-substituted by —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —SR₃₀, —SOR₃₁, —SO₂R₃₂ or by C₃-C₆cycloalkyl; or
each R₁ independently is phenyl which may in turn be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or, when Q is a group Q₁, Q₂, Q₃ or Q₅ to adjacent R₁ substituents may together form a C₁-C₇alkylene bridge which may be interrupted by from 1 to 3 hetero atoms selected from oxygen, nitrogen and sulfur and which may be mono- to penta-substituted by halogen or by C₁-C₆alkyl or mono-, di- or tri-substituted by C₁-C₆alkoxy, the total number of ring atoms being at least 5 and at most 9; or,
when Q is a group Q₁, Q₂, Q₃ or Q₅, two adjacent R₁ substituents may together form a C₂-C₇alkenylene bridge which may be interrupted by from 1 to 3 hetero atoms selected from oxygen, nitrogen and sulfur and which may be mono- to penta-substituted by halogen or by C₁-C₆alkyl or mono-, di- or tri-substituted by C₁-C₆alkoxy, the total number of ring atoms being at least 5 and at most 9;
R₃ or R₄ are each independently of the other hydrogen, halogen, —CN, C₁-C₄alkyl or C₁-C₄alkoxy; or
R₅ is hydrogen or C₁-C₈alkyl;
R₆ is hydrogen, C₁-C₈alkyl, C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₅ and R₆ together are a C₂-C₅alkylene chain which may be interrupted by an oxygen or sulfur atom;
R₇ is hydrogen, C₁-C₈alkyl, C₃-C₈alkenyl or C₃-C₈alkynyl, or is C₁-C₈alkyl, C₃-C₈alkenyl or C₃-C₈alkynyl mono- to penta-substituted by halogen or mono-, di- or tri-substituted by C₁-C₄alkoxy or by phenyl, it being possible for phenyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₈ is hydrogen or C₁-C₈alkyl;
R₉ is hydrogen or C₁-C₈alkyl, or is C₁-C₈alkyl mono-, di- or tri-substituted by —COOH, C₁-C₈alkoxycarbonyl or by —CN, or
R₉ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₈ and R₉ together are C₂-C₅alkylene;
R₁₀ is hydrogen, C₁-C₄alkyl, C₁-C₄haloalkyl or C₃-C₆cycloalkyl;
R₁₁ is hydrogen, C₁-C₈alkyl, C₃-C₈alkenyl, C₃-C₈alkynyl, C₁-C₄haloalkyl or C₃-C₆haloalkenyl;
R₁₂ is hydrogen, C₁-C₄alkyl, C₁-Q₄haloalkyl or C₃-C₆cycloalkyl;
R₁₃ is hydrogen, C₁-C₈alkyl, C₃-C₈alkenyl or C₃-C₈alkynyl; or
R₁₃ is phenyl or phenyl-C₁-C₆alkyl, it being possible for the phenyl ring in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₈alkylthio, C₁-C₈alkylsulfinyl or by C₁-C₈alkylsulfonyl, or
R₁₃ is C₁-C₈alkyl mono- to penta-substituted by halogen or mono-, di- or tri-substituted by CN, C₁-C₆alkylamino, di(C₁-C₆alkyl)amino or by C₁-C₄alkoxy;
R₁₄ is hydrogen, C₁-C₈alkyl, C₃-C₈alkenyl or C₃-C₈alkynyl, or is C₁-C₈alkyl mono- to penta-substituted by halogen or mono-, di- or tri-substituted by —CN or by C₁-C₄alkoxy;
R₁₅, R₁₈ and R₁₇ are each independently of the others C₁-C₈alkyl, C₃-C₈alkenyl or C₃-C₈alkynyl, or C₁-C₈alkyl mono- to penta-substituted by halogen or mono-, di- or tri-substituted by —CN or by C₁-C₄alkoxy;
R₁₈ is hydrogen or C₁-C₈alkyl;
R₁₉ is hydrogen, C₁-C₈alkyl, C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₈ and R₁₉ together are a C₂-C₅alkylene chain which may be interrupted by an oxygen or sulfur atom;
R₂₀ is hydrogen, C₁-C₈alkyl, C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio,
C₁-C₄alkylsulfinyl or by C₁-C₄alkylsulfonyl;
R₂₁ is hydrogen or C₁-C₈alkyl;
R₂₂ is hydrogen or C₁-C₈alkyl, or is C₁-C₈alkyl mono-, di- or tri-substituted by —COOH, C₁-C₈alkoxycarbonyl or by —CN, or
R₂₂ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₂₁ and R₂₂ together are C₂-C₅alkylene;
R₂₃ is hydrogen, C₁-C₄alkyl, C₁-C₄haloalkyl or C₃-C₆cycloalkyl;
R₂₄ is hydrogen, C₁-C₄alkyl, C₁-C₄haloalkyl or C₃-C₆cycloalkyl;
R₂₅ is hydrogen, C₁-C₈alkyl, C₃-C₈alkenyl, C₃-C₈alkynyl, C₁-C₄haloalkyl or C₃-C₆haloalkenyl;
R₂₆ is hydrogen or C₁-C₈alkyl;
R₂₇ is hydrogen or C₁-C₈alkyl, or is C₁-C₈alkyl mono-, di- or tri-substituted by —COOH, C₁-C₈-alkoxycarbonyl or by —CN, or
R₂₇ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₂₆ and R₂₇ together are C₂-C₅alkylene;
R₂₈ is hydrogen or C₁-C₈alkyl;
R₂₉ and R₃₀ are each independently of the other hydrogen, C₁-C₈alkyl, C₃-C₈alkenyl or C₃-C₈alkynyl, or C₁-C₈alkyl mono- to penta-substituted by halogen or mono-, di- or tri-substituted by —CN or by C₁-C₄alkoxy;
R₃₁ and R₃₂ are each independently of the other C₁-C₈alkyl, C₃-C₈alkenyl or C₃-C₈alkynyl, or C₁-C₈alkyl mono- to penta-substituted by halogen or mono-, di- or tri-substituted by —CN or, by C₁-C₄alkoxy;
m is 0, 1, 2, 3 or 4;
each R₂ independently is halogen, —CN, —SCN, —OCN, —N₃, —SF₅, —NO₂, —NR₃₃R₃₄, —CO₂R₃₅, —CONR₃₆R₃₇, —C(R₃₈)═NOR₃₉, —COR₄₀, —OR₄₁, —SR₄₂, —SOR₄₃, —SO₂R₄₄, —OSO₂R₄₅, —N([CO]ₚR₄₆)COR₄₇, —N(OR₅₄)COR₅₅, —N(R₅₆)SO₂R₅₇, —N(SO₂R₅₈)SO₂R₅₉, —N═C(OR₆₀)R₆₁, —CR₆₂(OR₆₃)OR₆₄, —OC(O)NR₆₅R₆₆, —SC(O)NR₆₇R₆₈, —OC(S)NR₆₉R₇₀ or —N-phthalimide; or
R₂ is a 5- to 7-membered heterocyclic ring system which may be aromatic or partially or fully saturated and may contain from 1 to 4 hetero atoms selected from nitrogen, oxygen and sulfur, it being possible for that heterocyclic ring system in turn to be mono- to penta-substituted by halogen or by C₁-C₄alkyl or mono-, di- or tri-substituted by C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or by C₁-C₆alkylsulfonyl;
R₃₃ is hydrogen or C₁-C₈alkyl; and
R₃₄ is hydrogen, C₁-C₈alkyl, C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₃₃ and R₃₄ together are a C₂-C₅alkylene chain which may be interrupted by an oxygen or sulfur atom;
R₃₅ is hydrogen, C₁-C₈alkyl, C₃-C₈alkenyl or C₃-C₈alkynyl, or is C₁-C₈alkyl, C₃-C₈alkenyl or C₃-C₈alkynyl mono- to penta-substituted by halogen or mono-, di- or tri-substituted by C₁-C₄alkoxy or by phenyl, it being possible for phenyl in turn to be mono- to penta-substituted by halogen or by C₁G₄alkyl or mono-, di- or tri-substituted by C₁-C₄haloalkyl; C₁-C₄alkoxy, —CN, —NO₂, C₁-C₄alkylthio, C₁-C₄alkylsulfinyl or by C₁-C₄alkylsulfonyl;
R₃₆is hydrogen or C₁-C₈alkyl;
R₃₇ is hydrogen or C₁-C₈alkyl, or is C₁-C₈alky mono-, di- or tri-substituted by —COOH, C₁-C₈-alkoxycarbonyl or by —CN, or
R₃₇ is C₃-C₈alkenyl, C₃-C₈alkynyl, phenyl or benzyl, it being possible for phenyl and benzyl int turn to be mono- to penta-substituted by halogen or by C₁-C₄alkyl or mono-, dl- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₃₆ and R₃₇ together are C₃-C₅alkylene;
R₃₈ is hydrogen, C₁-C₄alkyl, C₁-C₄haloalkyl or C₃-C₆cycloalkyl;
R₃₉ is hydrogen, C₁-C₈alkyl, C₃-C₈alkenyl, C₃-C₈alkynyl, C₁-C₄haloalkyl or C₃-C₆haloalkenyl;
R₄₀ is hydrogen, C₁-C₄alkyl, C₁-C₄haloalkyl, C₁-C₈alkylthio, —C(O)—C(O)OC₁-C₄alkyl or C₃-C₆-cycloalkyl;
R₄₁ is hydrogen, C₁-C₈alkyl, C₃-C₈alkenyl, C₃-C₈alkynyl, C₁-C₆alkoxy-C₁-C₆alkyl, C₁-C₈alkylcarbonyl, C₁-C₈alkoxycarbonyl, C₃-C₈alkenyloxycarbonyl, C₁-C₆alkoxy-C₁-C₆alkoxycarbonyl, C₁-C₆alkylthio-C₁-C₆alkyl, C₁-C₆alkylsulfinyl-C₁-C₆alkyl or C₁-C₆alkylsulfonyl-C₁-C₆alkyl; or
R₄₁ is phenyl or phenyl-C₁-C₆alkyl, it being possible for the phenyl ring in turn to be mono- to penta-substituted by halogen or by C₁-C₄alkyl or mono-, di- or tri-substituted by C₁-C₄haloalkyl, C₁-C₄alkoxy, —CN, —NO₂ or by —S(O)₂C₁-C₈alkyl, or
R₄₁ is C₁-C₈alkyl mono- to penta-substituted by halogen or mono-, di- or tri-substituted by —COOH, C₁-C₈alkoxycarbonyl, C₁-C₆alkylamino, di(C₁-C₆alkyl)amino or by —CN;
R₄₂ is hydrogen, C₁-C₈alkyl, C₃-C₈alkenyl or C₃-C₈alkynyl, or is C₁-C₈alkyl mono- to penta-substituted by halogen or mono-, di- or tri-substituted by —CN or by C₁-C₄alkoxy;
R₄₃ and R₄₄ are each independently of the other C₁-C₈alkyl, C₃-C₈alkenyl or C₃-C₈alkynyl, or C₁-C₈alkyl mono- to penta-substituted by halogen or mono-, di- or tri-substituted by —CN or by C₁-C₄alkoxy;
R₄₅ is C₁-C₈alkyl, C₁-C₈alkyl mono- to penta-substituted by halogen or mono-, di- or tri-substituted by —CN or by C₁-C₄alkoxy, or is C₃-C₈alkenyl or C₃-C₈alkynyl, or
R₄₅ is phenyl, it being possible for the phenyl ring to be mono- to penta-substituted by halogen or by C₁-C₄alkyl or mono-, di- or tri-substituted by C₁-C₄haloalkyl, C₁-C₄alkoxy, —CN, NO₂, C₁-C₈alkylthio, C₁-C₈alkylsulfinyl or by C₁-C₈alkylsulfonyl;
R₄₆ is hydrogen, C₁-C₈alkyl, C₃-C₈alkenyl, C₃-C₈alkynyl or C₁-C₄haloalkyl;
R₄₇ is hydrogen, C₁-C₈alkyl, C₁-C₄alkoxy, C₃-C₈alkenyl or C₃-C₈alkynyl, or is C₁-C₈alkyl mono- to penta-substituted by halogen or mono-, di- or tri-substituted by —CN, C₁-C₄alkoxy, C₁-C₈alkoxycarbonyl, —NH₂, C₁-C₄alkylamino, di(C₁-C₄alkyl)amino, —NR₄₈COR₄₉, —NR₅₀SO₂R₅₁ or by —NR₅₂CO₂R₅₃, or
R₄₇ is phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
p is 0 or 1;
R₄₈, R₄₉, R₅₀, R₅₁, R₅₂ and R₅₃ are each independently of the others hydrogen, C₁-C₈alkyl, phenyl, benzyl or naphthyl, it being possible for the three last mentioned aromatic radicals in turn to be mono- to penta-substituted by halogen or by C₁-C₈alkyl or mono-, di- or tri-substituted by C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₅₄ and R₅₅ are each independently of the other hydrogen, C₁-C₈alkyl, or phenyl which may in turn be mono- to penta-substituted by halogen or by C₁-C₄alkyl or mono-, di- or tri-substituted by C₁-C₄haloalkyl, C₁-C₄alkoxy, —CN, —NO₂, C₁-C₈alkylthio, C₁-C₈alkylsulfinyl or by C₁-C₈alkylsulfonyl;
R₅₆ is hydrogen, C₁-C₈alkyl, C₁-C₄haloalkyl, C₁-C₄alkoxy, C₃-C₈alkenyl, C₃-C₈alkynyl or benzyl, it being possible for benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₈alkylthio, C₁-C₈alkylsulfinyl or by C₁-C₈alkylsulfonyl;
R₅₇ is C₁-C₈alkyl, C₁-C₄haloalkyl, phenyl, benzyl or naphthyl, it being possible for the last three aromatic rings to be mono- to penta-substituted by halogen or by C₁-C₄alkyl or mono-, di- or tri-substituted by C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₅₈ and R₅₉ are each independently of the other C₁-C₈alkyl, C₃-C₈alkenyl, C₃-C₈alkynyl, phenyl, benzyl or naphthyl, it being possible for the last three aromatic rings to be mono- to penta-substituted by halogen or by C₁-C₄alkyl or mono-, di- or tri-substituted by C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₆₀ and R₆₁ are each independently of the other hydrogen or C₁-C₆alkyl;
R₆₂, R₆₃ and R₆₄ are each independently of the others hydrogen or C₁-C₈alkyl, or
R₆₃ and R₆₄ together form a C₂-C₅alkylene bridge;
R₆₅, R₆₆, R₆₇, R₆₈, R₆₉ and R₇₀ are each independently of the others hydrogen or C₁-C₈alkyl, or
R₆₅ and R₆₆, or R₆₇ and R₆₈, or R₆₉ and R₇₀ in each case together form a C₂-C₅alkylene bridge; or
each R₂ independently is C₁-C₂alkyl, or is C₁-C₈alkyl mono- to penta-substitution by halogen or mono-, di- or tri-substituted by —CN, —N₃, —SCN, —NO₂, —NR₇₁R₇₂, —CO₂R₇₃, —CONR₇₄R₇₅, —COR₇₆, —C(R₇₇)═NOR₇₈, —C(S)NR₇₉R₈₀, —C(C₁-C₄alkylthio)═NR₈₁, —OR₈₂, —SR₈₃, —SOR₈₄, —SO₂R₈₅, —O(SO₂)R₈₆, —N(R₈₇)CO₂R₈₈, —N(R₈₉)COR₉₀, —S⁺(R₉₁)₂, —N⁺(R₉₂)₃, —Si(R₉₃)₃ or by C₃-C₆cycloalkyl; or
each R₂ independently is C₁-C₈alkyl substituted by a 5- to 7-membered heterocyclic ring system which may be aromatic or partially or fully saturated and may contain from 1 to 4 hetero atoms selected from oxygen, nitrogen and sulfur, it being possible for that heterocyclic ring system in turn to be mono- to penta-substituted by halogen or by C₁-C₄alkyl or mono-, di- or tri-substituted by C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or by C₁-C₆alkylsulfonyl; or
each R₂ independently is C₂-C₈alkenyl, or is C₂-C₈alkenyl mono- to penta-substituted by halogen or mono-, di- or tri-substituted by —CN, —NO₂, —CO₂R₉₄, —CONR₉₅R₉₆, —COR₉₇, —C(R₉₈)═NOR₉₉, —C(S)NR₁₀₀R₁₀₁, —C(C₁-C₄alkylthio)═NR₁₀₂, —OR₁₀₃, —Si(R₁₀₄)₃ or by C₃-C₆-cycloalkyl; or
each R₂ independently is C₂-C₈alkynyl, or is C₂-C₈alkynyl mono- to penta-substituted by halogen or mono-, di- or tri-substituted by —CN, —CO₂R₁₀₅, —CONR₁₀₆R₁₀₇, —COR₁₀₈, —C(R₁₀₉)═NOR₁₁₀, —C(S)NR₁₁₁R₁₁₂, —C(C₁-C₄alkylthio)═NR₁₁₃, —OR₁₁₄, —Si(R₁₁₅)₃ or by C₃-C₆cycloalkyl; or
each R₂ independently is C₃-C₆cycloalkyl, or is C₃-C₆cycloalkyl mono- to penta-substituted by halogen or mono-, di- or tri-substituted by —CN, —CO₂R₁₁₆, —CONR₁₁₇R₁₁₈, —COR₁₁₉, —C(R₁₂₀)═NOR₁₂₁, —C(S)NR₁₂₂R₁₂₃ or by —C(C₁-C₄alkylthio)═NR₁₂₄; or
two adjacent R₂ substituents together form a C₁-C₇alkylene bridge which may be interrupted by from 1 to 3 hetero atoms selected from oxygen, nitrogen and sulfur and may be mono- to penta-substituted by halogen or by C₁-C₆alkyl or mono-, di- or tri-substituted by C₁-C₆alkoxy, the total number of ring atoms being at least 5 and at most 9; or
two adjacent R₂ substituents together form a C₂-C₇alkenylene bridge which may be interrupted by from 1 to 3 hetero atoms selected from oxygen, nitrogen and sulfur and may be mono- to penta-substituted by halogen or by C₁-C₆alkyl or mono-, di- or tri-substituted by C₁-C₆alkoxy, the total number of ring atoms being at least 5 and at most 9;
R₇₁ is hydrogen or C₁-C₈alkyl;
R₇₂ is hydrogen, C₁-C₈alkyl, C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₇₁ and R₇₂ together are a C₂-C₅alkylene obain which may be interrupted by an oxygen or sulfur atom;
R₇₃ is hydrogen, C₁-C₈alkyl, C₃-C₈alkenyl or C₃-C₈alkynyl, or is C₁-C₈alkyl, C₃-C₈alkenyl or C₃-C₈alkynyl mono- to penta-substituted by halogen or mono-, di- or tri-substituted by C₁-C₄alkoxy or by phenyl, it being possible for henyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₇₄ is hydrogen or C₁-C₈alkyl;
R₇₅ is hydrogen, C₁-C₈alkyl or C₃-C₈cycloalkyl, or is C₁-C₈alkyl mono-, di- or tri-substituted by —COOH, C₁-C₈alkoxycarbonyl, C₁-C₆alkoxy or by —CN; or
R₇₅ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio; C₁-C₄alkylsufinyl; or
R₇₄ and R₇₅ together are a C₂-C₅alkylene chain which may be interrupted by an oxygen or sulfur atom;
R₇₆ is hydrogen, C₁-C₄alkyl, C₁-C₄haloalkyl or C₃-C₆cycloalkyl;
R₇₇ is hydrogen, C₁-C₄alkyl, C₁-C₄haloalkyl or C₃-C₆cycloalkyl;
R₇₈ is hydrogen, C₁-C₈alkyl, C₃-C₈alkenyl, C₃-C₈alkynyl, C₁-C₄haloalkyl or C₃-C₆haloalkenyl; and
R₇₉ is hydrogen or C₁-C₈alkyl;
R₈₀ is hydrogen or C₁-C₈alkyl, or is C₁-C₈alkyl mono-, di- or tri-substituted by —COOH, C₁-C₈-alkoxycarbonyl or by —CN; or
R₈₀ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₇₉ and R₈₀ together are C₂-C₅alkylene;
R₈₁ is hydrogen or C₁-C₈alkyl;
R₈₂ is —Si(C₁-C₆alkyl)₃, C₃-C₈alkenyl or C₃-C₈alkynyl, or is C₁-C₈alkyl which is mono- to penta-substituted by halogen or mono-, di- or tri-substituted by —CN, —NH₂, C₁-C₆alkylamino, di(C₁-C₆alkyl)amino or by C₁-C₄alkoxy;
R₈₃ is hydrogen, C₁-C₈alkyl, C₃-C₈alkenyl or C₃-C₈alkynyl, or is C₁-C₈alkyl which is mono- to penta-substituted by halogen or mono-, di- or tri-substituted by —CN, —NH₂, C₁-C₆alkylamino, di(C₁-C₆alkyl)amino or by C₁-C₄alkoxy;
R₈₄, R₈₅ and R₈₆ are each independently of the others C₁-C₆alkyl, C₃-C₈-alkenyl or C₃-C₈-alkynyl, or C₁-C₈alkyl which is mono- to penta-substituted by halogen or mono-, di- or tri-substituted by —CN or by C₁-C₄alkoxy;
R₈₇ and R₈₉ are each independently of the other hydrogen; C₁-C₈alkyl or C₁-C₈alkoxy;
R₈₈ is C₁-C₈alkyl;
R₉₀ is hydrogen or C₁-C₈alkyl;
R₉₁ is C₁-C₄alkyl;
R₉₂ and R₉₃ are each independently of the other C₁-C₆alkyl;
R₉₄ is hydrogen or is C₁-C₈alkyl, C₃-C₈alkenyl or C₃-C₈alkynyl, each of which may be mono- to penta-substituted by halogen or mono-, di- or tri-substituted by C₁-C₄alkoxy or by phenyl, it being possible for phenyl in turn to be mono- to penta-substituted by halogen or by C₁-C₄-alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₉₅ is hydrogen or C₁-C₈alkyl;
R₉₆ is hydrogen or C₁-C₈alkyl, or is C₁-C₈alkyl mono-, di- or tri-substituted by —COOH, C₁-C₃alkoxycarbonyl or by —CN; or
R₉₆ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₉₅ and R₉₆ together are C₂-C₅alkylene;
R₉₇ and R₉₈ are each independently of the other hydrogen, C₁-C₄alkyl, C₁-C₄haloalkyl or C₃-C₆cycloalkyl;
R₉₉ is hydrogen, C₁-C₈alkyl, C₃-C₈alkenyl, C₃-C₈alkynyl, C₁-C₄haloalkyl or C₃-C₈haloalkenyl;
R₁₀₀ is hydrogen or C₁-C₈alkyl;
R₁₀₁ is hydrogen or C₁-C₈alkyl, or is C₁-C₈alkyl mono-, di- or tri-substituted by —COOH, C₁-C₈-alkoxycarbonyl or by —CN; or
R₁₀₁ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₀₀ and R₁₀₁ together are C₂-C₅alkylene;
R₁₀₂ is hydrogen or C₁-C₈alkyl;
R₁₀₃ is hydrogen, C₁-C₈alkyl, —Si(C₁-C₆alkyl)₃, C₃-C₈alkenyl or C₃-C₈alkynyl;
R₁₀₄ is C₁-C₆alkyl; R₁₀₅ is hydrogen or is C₃-C₈alkyl, C₃-C₈alkenyl or C₃-C₈alkynyl, each of which may be mono- to penta-substituted by halogen or mono-, di- or tri-substituted by C₁-C₄alkoxy or by phenyl it being possible for phenyl in turn to be mono- to penta-substituted by halogen or by C₁-C₄-alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₁₀₆ is hydrogen or C₁-C₈alkyl;
R₁₀₇ is hydrogen or C₁-C₈alkyl, or is C₁-C₈alkyl mono-, di- or tri-substituted by —COOH, C₁-C₈alkoxycarbonyl or by —CN, or
R₁₀₇ is C₃-C₈alkenyl, C₃-C₈alkynyl, phenyl or benzyl, it being possible for phenyl and benzyl in turn to be mono- to penta-substituted by halogen or by C₁-C₄alkyl or mono; di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₀₆ and R₁₀₇ together are C₂-C₅alkylene;
R₁₀₈ is hydrogen, C₁-C₄alkyl, C₁-C₄haloalkyl or C₃-C₆cycloalkyl;
R₁₀₉ is hydrogen, C₁-C₄alkyl, C₁-C₄haloalkyl or C₃-C₆cycloalkyl;
R₁₁₀ is hydrogen, C₁-C₈alkyl, C₃-C₈alkenyl, C₃-C₈alkynyl, C₁-C₄haloalkyl or C₃-C₆haloalkenyl;
R₁₁₁ is hydrogen or C₁-C₈alkyl;
R₁₁₂ is hydrogen or C₁-C₈alkyl, or is C₁-C₈alkyl mono-, di- or tri-substituted by —COOH, C₁-C₈alkoxycarbonyl or by —CN; or
R₁₁₂ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₁₁ and R₁₁₂ together are C₂-C₅alkylene;
R₁₁₃ is hydrogen or C₁-C₈alkyl;
R₁₁₄ is hydrogen, C₁-C₈alkyl, —Si(C₁-C₆alkyl)₃, C₃-C₈alkenyl or C₃-C₈alkynyl;
R₁₁₅ is C₁-C₆alkyl;
R₁₁₆ is hydrogen or is C₁-C₈alkyl, C₃-C₈alkenyl or C₃-C₈alkynyl, each of which may be mono- to penta-substituted by halogen or mono-, di- or tri-substituted by C₁-C₄alkoxy or by phenyl, it being possible for phenyl in turn to be mono- to penta-substituted by halogen or by C₁-C₄-alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl;
R₁₁₇ is hydrogen or C₁-C₈alkyl;
R₁₁₈ is hydrogen or C₁-C₈alkyl, or is C₁-C₈alkyl mono-, di- or tri-substituted by —COOH, C₁-C₈alkoxycarbonyl or by —CN; or R₁₁₈ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylsulfinyl or by C₁-C₄alkylsulfonyl; or
R₁₁₇ and R₁₁₈ together are C₂-C₅alkylene;
R₁₁₉ is hydrogen, C₁-C₄alkyl, C₁-C₄haloalkyl or C₃-C₆cycloakyl;
R₁₂₀ is hydrogen, C₁-C₄alkyl, C₁-C₄haloalkyl or C₃-C₆cycloalkyl;
R₁₂₁ is hydrogen, C₁-C₈alkyl, C₃-C₈alkenyl, C₃-C₈alkynyl, C₁-C₄haloalkyl or C₃-C₆haloalkenyl;
R₁₂₂ is hydrogen or C₁-C₈alkyl;
R₁₂₃ is hydrogen or C₁-C₈alkyl, or is C₁-C₈alkyl mono-, di- or tri-substituted by —COOH, C₁-C₈alkoxycarbonyl or by —CN; or
R₁₂₃ is C₃-C₈alkenyl, C₃-C₈alkynyl, phenyl or benzyl, it being possible for phenyl and benzyl in turn to be mono- to penta-substituted by halogen or by C₁-C₄alkyl or mono-, di- or tri-substituted by C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₂₂ and R₁₂₃ together are C₂-C₅alkylene; and
R₁₂₄ is hydrogen or C₁-C₈alkyl, or an agrochemically acceptable salt or any stereoisomer or tautomer of a compound of formula I.

2. A compound of formula I wherein each R₁ independently is halogen, —CN, —SCN, —SF₅, —NO₂, —NR₅R₆, —CO₂R₇, —CONR₈R₉, —C(R₁₀)═NOR₁₁, —COR₁₂, —OR₁₃, —SR₁₄, —SOR₁₅, —SO₂R₁₆, —OSO₂R₁₇, C₁-C₈alkyl, C₂-C₈alkenyl, C₂-C₈alkynyl or C₃-C₆cycloalkyl; or is C₁-C₈alkyl, C₂-C₈alkenyl or C₂-C₈alkynyl mono-, di- or tri-substituted by halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —OR₂₉, —SR₃₀, —SOR₃₁, —SO₂R₃₂ or by C₃-C₆cycloalkyl; or
each R₁ independently is C₃-C₆cycloalkyl mono-, di- or tri-substituted by halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —SR₃₀, —SOR₃₁, —SO₂R₃₂ or by C₃-C₆cycloalkyl; or
each R₁ independently is phenyl which may in turn be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or,
when Q is a group Q₁, Q₂, Q₃ or Q₅, two adjacent R₁ substituents together may form a C₁-C₇alkylene bridge which may be interrupted by from 1 to 3 hetero atoms selected from oxygen, nitrogen, and sulfur and may be mono-, di- or tri-substituted by halogen, C₁-C₆alkyl or by C₁-C₆alkoxy, the total number of ring atoms being at least 5 and at most 9; or, when Q is a group Q₁, Q₂, Q₃ or Q₅, two adjacent R₁ substitutents together may form a C₂-C₇alkenylene bridge which may be interrupted by from 1 to 3 hetero atoms selected from oxygen, nitrogen and sulfur and may be mono-, di- or tri-substituted by halogen, C₁-C₆alkyl or by C₁-C₆alkoxy, the total number of ring atoms being at least 5 and at most 9;
R₃ or R₄ are each independently of the other hydrogen, halogen, —CN, C₁-C₄alkyl or C₁-C₄alkoxy; or
R₃ and R₄ together are C₂-C₅alkylene;
R₅ is hydrogen or C₁-C₈alkyl;
R₆ is hydrogen, C₁-C₈alkyl, C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₅ and R₆ together are a C₂-C₅alkylene chain which, may be interrupted by an oxygen or sulfur atom;
R₇ is hydrogen, C₁-C₈alkyl, C₃-C₈alkenyl or C₃-C₈alkynyl, or is C₁-C₈alkyl, C₃-C₈alkenyl or C₃-C₈alkynyl mono-, di- or tri-substituted by halogen, C₁-C₄alkoxy or by phenyl, it being possible for phenyl in turn to be mono-, di- or tri-substituted by halogen, C₁-C₄alkyl, C₁-C₄-haloalkyl, C₁-C₄alkoxy, —CN, —NO₂, C₁-C₄alkylthio, C₁-C₄alkylsulfinyl or by C₁-C₄alkylsulfonyl;
R₈ is hydrogen or C₁-C₈alkyl;
R₉ is hydrogen or C₁-C₈alkyl, or is C₁-C₈alkyl mono-, di- or tri-substituted by —COOH, C₁-C₈alkoxycarbonyl or by —CN, or
R₉ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₈ and R₉ together are C₂-C₅alkylene;
R₁₀ is hydrogen, C₁-C₄alkyl, C₁-C₄haloalkyl or C₃-C₆cycloalkyl;
R₁₁ is hydrogen, C₁-C₈alkyl, C₃-C₈alkenyl, C₃-C₈alkynyl, C₁-C₄haloalkyl or C₃-C₆haloalkenyl;
R₁₂ is hydrogen, C₁-C₄alkyl, C₁-C₄haloalkyl or C₃-C₆cycloalkyl;
R₁₃ is hydrogen, C₁-C₈alkyl, C₃-C₈alkenyl or C₃-C₈alkynyl; or
R₁₃ is phenyl or phenyl-C₁-C₆alkyl, it being possible for the phenyl ring in turn to be mono-, di- or tri-substituted by halogen, C₁-C₄alkyl, C₁-C₄haloalkyl, C₁-C₄alkoxy, —CN, —NO₂, C₁-C₈-alkylthio, C₁-C₈alkylsulfinyl or by C₁-C₈alkylsulfonyl; or
R₁₃ is C₁-C₈alkyl mono-, di- or tri-substituted by halogen, —CN, C₁-C₆alkylamino, di(C₁-C₆-alkyl)amino or by C₁-C₄alkoxy; R₁₄ is hydrogen, C₁-C₈alkyl, C₃-C₈alkenyl or C₃-C₈alkynyl, or is C₁-C₈alkyl mono-, di- or tri-substituted by halogen, —CN or by C₁-C₄alkoxy;
R₁₅, R₁₆ and R₁₇ are each independently of the others C₁-C₈alkyl, C₃-C₈alkenyl or C₃-C₈-alkynyl, or C₁-C₈alkyl mono-, di- or tri-substituted by halogen; —CN or by C₁-C₄alkoxy;
R₁₈ is hydrogen or C₁-C₈alkyl;
R₁₉ is hydrogen, C₁-C₈alkyl, C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₈ and R₁₉ together are a C₂-C₅alkylene chain which may be interrupted by an oxygen or sulfur atom;
R₂₀ is hydrogen, C₁-C₈alkyl, C₃-C₈alkenyl, C₃-C₈alkynyl, phenyl or benzyl, it being possible for phenyl and benzyl in turn to be mono-, di- or tri-substituted by halogen C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl;
R₂₁ is hydrogen or C₁-C₈alkyl;
R₂₂ is hydrogen or C₁-C₈alkyl, or is C₁-C₈alkyl mono-, di- or tri-substituted by —COOH, C₁-C₈alkoxycarbonyl or by —CN, or
R₂₂ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₂₁ and R₂₂ together are C₂-C₅alkylene;
R₂₃ is hydrogen, C₁-C₄alkyl, C₁-C₄haloalkyl or C₃-C₆cycloalkyl;
R₂₄ is hydrogen, C₁-C₄alkyl, C₁-C₄haloalkyl or C₃-C₆cycloalkyl;
R₂₅ is hydrogen, C₁-C₈alkyl, C₃-C₈alkenyl, C₃-C₈alkynyl, C₁-C₄haloalkyl or C₃-C₆haloalkenyl;
R₂₆ is hydrogen or C₁-C₈alkyl;
R₂₇ is hydrogen or C₁-C₈alkyl, or is C₁-C₈alkyl mono-, di- or tri-substituted by —COOH, C₁-C₈alkoxycarbonyl or by —CN, or
R₂₇ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₂₆ and R₂₇ together are C₂-C₅alkylene;
R₂₈ is hydrogen or C₁-C₈alkyl;
R₂₉ and R₃₀ are each independently of the other hydrogen, C₁-C₈alkyl, C₃-C₈alkenyl or C₃-C₈alkynyl, or C₁-C₆alkyl mono-, di- or tri-substituted by halogen, —CN or by C₁-C₄alkoxy; R₃₁ and R₃₂ are each independently of the other C₁-C₈alkyl, C₃-C₈alkenyl or C₃-C₈alkynyl, or C₁-C₈alkyl mono-, di- or tri-substituted by halogen, —CN or by C₁-C₄alkoxy;
m is 0, 1, 2, 3 or 4;
each R₂ independently is hydrogen, halogen, —CN, —SCN, —CON, —N₃, —SF₅, —NO₂, —NR₃₃R₃₄, —CO₂R₃₅, —CONR₃₆R₃₇, —C(R₃₈)═NOR₃₉, —COR₄₀, —OR₄₁; —SR₄₂, —SOR₄₃, —SO₂R₄₄, —OSO₂R₄₅, —N([CO]ₚR₄₆)COR₄₇, —N(OR₅₄)COR₅₅, —N(R₅₆)SO₂R₅₇, —N(SO₂R₅₈)SO₂R₅₉, —N═C(OR₆₀)R₆₁, —CR₆₂(OR₆₃)OR₆₄, —OC(O)NR₆₅R₆₆, —SC(O)NR₆₇R₆₈, —OC(S)NR₆₉R₇₀ or —N-phthalimide; or
R₂ is a 5- to 7-membered heterocylic ring system which may be aromatic or partially or fully saturated and may contain from 1 to 4 hetero atoms selected from nitrogen, oxygen and sulfur, it being possible for that heterocyclic ring system in turn to be mono-, di- or tri-substituted by halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or by C₁-C₆alkylsulfonyl;
R₃₃ is hydrogen or C₁-C₈alkyl; and
R₃₄ is hydrogen, C₁-C₈alkyl; C₃-C₈alkenyl, C₃-C₈alkynyl; phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₃₃ and R₃₄ together are a C₂-C₅alkylene chain which may be interrupted by an oxygen or sulfur atom;
R₃₅ is hydrogen, C₁-C₈alkyl, C₃-C₈alkenyl or C₃-C₈alkynyl, or is C₁-C₈alkyl, C₃-C₈alkenyl or C₃-C₈alkynyl mono-, di- or tri-substituted by halogen, C₁-C₄alkoxy or by phenyl, it being possible for phenyl in turn to be mono-, di- or tri-substituted by halogen, C₁-C₄alkyl, C₁-C₄-haloalkyl, C₁-C₄alkoxy, —CN, —NO₂, C₁-C₄alkylthio, C₁-C₄alkylsulfinyl or by C₁-C₄alkylsulfonyl;
R₃₆ is hydrogen or C₁-C₈alkyl;
R₃₇ is hydrogen or C₁-C₈alkyl, or is C₁-C₈alkyl mono-, di- or tri-substituted by —COOH, C₁-C₈alkoxycarbonyl or by —CN, or
R₃₇ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₃₆ and R₃₇ together are C₃-C₅alkylene;
R₃₈ is hydrogen, C₁-C₄alkyl, C₁-C₄haloalkyl or C₃-C₆cycloalkyl;
R₃₉ is hydrogen, C₁-C₈alkyl, C₃-C₈alkenyl, C₃-C₈alkynyl, C₁-C₄haloalkyl or C₃-C₆haloalkenyl;
R₄₀ is hydrogen, C₁-C₄alkyl, C₁-C₄haloalkyl, C₁-C₈alkylthio, —C(O)—C(O)OC₁-C₄alkyl or C₃-C₆cycloalkyl; R₄₁ is hydrogen, C₁-C₈alkyl, C₃-C₈alkenyl, C₃-C₈alkynyl, C₁-C₆alkoxy-C₁-C₆alkyl, C₁-C₈alkylcarbonyl, C₁-C₈alkoxycarbonyl, C₃-C₈alkenyloxycarbonyl, C₁-C₆alkoxy-C₁-C₆alkoxycarbonyl, C₁-C₆alkylthio-C₁-C₆alkyl, C₁-C₆alkylsulfinyl-C₁-C₆alkyl or C₁-C₆alkylsulfonyl-C₁-C₆alkyl; or
R₄₁ is phenyl or phenyl-C₁-C₆alkyl, it being possible for the phenyl ring in turn to be mono-, di- or tri-substituted by halogen, C₁-C₄alkyl, C₁-C₄haloalkyl, C₁-C₄alkoxy, —CN, —NO₂ or by —S(O)₂C₁-C₈alkyl, or
R₄₁ is C₁-C₈alkyl mono-, di- or tri-substituted by —COOH, C₁-C₈alkoxycarbonyl, C₁-C₆alkylamino, di(C₁-C₆alkyl)amino or by —CN;
R₄₂ is hydrogen, C₁-C₈alkyl, C₃-C₈alkenyl or C₃-C₈alkynyl, or is C₁-C₈alkyl mono-, di- or tri-substituted by halogen, —CN or by C₁-C₄alkoxy;
R₄₃ and R₄₄ are each independently of the other C₁-C₈alkyl, C₃-C₈alkenyl or C₃-C₈alkynyl, or C₁-C₈alkyl mono-, di- or tri-substituted by halogen, —CN or by C₁-C₄alkoxy;
R₄₅ is C₁-C₈alkyl, C₁-C₈-alkyl mono-, di- or tri-substituted by halogen, —CN or by C₁-C₄alkoxy, or is C₃-C₈alkenyl or C₃-C₈alkynyl, or
R₄₅ is phenyl, it being possible for the phenyl ring to be mono-, di- or tri-substituted by halogen, C₁-C₄alkyl, C₁-C₄haloalkyl, C₁-C₄alkoxy, —CN, NO₂, C₁-C₈alkylthio, C₁-C₈alkylsulfinyl or by C₁-C₈alkylsulfonyl;
R₄₆ is hydrogen, C₁-C₈alkyl, C₃-C₈alkenyl, C₃-C₈alkynyl or C₁-C₄haloalkyl;
R₄₇ is hydrogen, C₁-C₈alkyl, C₁-C₄alkoxy, C₃-C₈alkenyl or C₃-C₈alkynyl, or is C₁-C₈alkyl mono-, di- or tri-substituted by halogen, —CN, C₁-C₄alkoxy, C₁-C₈alkoxycarbonyl, —NH₂, C₁-C₄alkylamino, di(C₁-C₄alkyl)amino, —NR₄₈COR₄₉, —NR₅₀SO₂R₅₁ or by —NR₅₂CO₂R₅₃, or R₄₇ is phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl;
p is 0 or 1;
R₄₈, R₄₉, R₅₀, R₅₁, R₅₂ and R₅₃ are each independently of the others hydrogen, C₁-C₈alkyl, phenyl, benzyl or naphthyl, it being possible for the three last-mentioned aromatic radicals in turn to be mono-, di- or tri-substituted by halogen, C₁-C₈alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₅₄ and R₅₅ are each independently of the other hydrogen, C₁-C₈alkyl, or phenyl which may in turn be mono-, di- or tri-substituted by halogen, C₁-C₄alkyl, C₁-C₄haloalkyl, C₁-C₄alkoxy, —CN, —NO₂, C₁-C₈alkylthio, C₁-C₈alkylsulfinyl or by C₁-C₈alkylsulfonyl;
R₅₆ is hydrogen, C₁-C₈alkyl, C₁-C₄haloalkyl, C₁-C₄alkoxy, C₃-C₈alkenyl, C₃-C₈alkynyl or benzyl, it being possible for benzyl in turn to be mono-, di- or tri-substituted by halogen, C₁-C₄alkyl, C₁-C₄haloalkyl, C₁-C₄alkoxy, —CN, —NO₂, C₁-C₈alkylthio, C₁-C₈alkylsulfinyl or by C₁-C₈alkylsulfonyl;
R₅₇ is C₁-C₈alkyl, C₁-C₄haloalkyl, phenyl, benzyl or naphthyl, it being possible for the last three aromatic rings to be mono-, di- or tri-substituted by halogen, C₁-C₄alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₅₈ and R₅₉ are each independently of the other C₁-C₈alkyl, C₃-C₈alkenyl, C₃-C₈alkynyl, phenyl, benzyl or naphthyl, it being possible for the last three aromatic rings to be mono-, di- or tri-substituted by halogen, C₁-C₄alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or by C₁-C₄alkylsulfonyl;
R₆₀ and R₆₁ are each independently of the other hydrogen or C₁-C₆alkyl;
R₆₂, R₆₃ and R₆₄ are each independently of the others hydrogen or C₁-C₈alkyl, or
R₆₃ and R₆₄ together form a C₂-C₅alkylene bridge;
R₆₅, R₆₆, R₆₇, R₆₈, R₆₉ and R₇₀ are each independently of the others hydrogen or C₁-C₈alkyl, or
R₆₅ and R₆₆, or R₆₇ and R₆₈, or R₆₉ and R₇₀ in each case together form a C₂-C₅alkylene bridge; or
each R₂ independently is C₁-C₈alkyl, or is C₁-C₈alkyl mono-, di- or tri-substituted by halogen, —CN, —N₃, —SCN, —NO₂, —NR₇₁R₇₂, —CO₂R₇₃, —CONR₇₄R₇₅, —COR₇₆, —C(R₇₇)═NOR₇₈, —C(S)NR₇₉R₈₀, —C(C₁-C₄alkylthio)═NR₈₁, —OR₈₂, —SR₈₃, —SOR₈₄, —SO₂R₈₅, —O(SO₂)R₈₆, —N(R₈₇)CO₂R₈₈, —N(R₈₉)COR₉₀, —S⁺(R₉₁)₂, —N⁺(R₉₂)₃, —Si(R₉₃)₃ or by C₃-C₆cycloalkyl; or
each R₂ independently is C₁-C₈alkyl substituted by a 5- to 7-membered heterocyclic ring system which may be aromatic or partially or fully saturated and may contain from 1 to 4 hetero atoms selected from nitrogen, oxygen and sulfur, it being possible for that hetero-cyclic ring system in turn to be mono-, di- or tri-substituted by halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or by C₁-C₆alkylsulfonyl; or
each R₂ independently is C₂-C₈alkenyl, or is C₂-C₈alkenyl mono-, di- or tri-substituted by —CN, —NO₂, —CO₂R₉₄, —CONR₉₅R₉₆, —COR₉₇, —C(R₉₈)═NOR₉₉, —C(S)NR₁₀₀R₁₀₁, —C(C₁-C₄alkylthio)═NR₁₀₂, —OR₁₀₃, —Si(R₁₀₄)₃ or by C₃-C₆cycloalkyl; or
each R₂ independently is C₂-C₈alkynyl, or is C₂-C₈alkynyl mono-, di- or tri-substituted by halogen, —CN, —CO₂R₁₀₅, —CONR₁₀₆R₁₀₇, —COR₁₀₈, —C(R₁₀₉)═NOR₁₁₀, —C(S)NR₁₁₁R₁₁₂, —C(C₁-C₄alkylthio)═NR₁₁₃, —OR₁₁₄, —Si(R₁₁₅)₃ or by C₃-C₆cycloalkyl; or each R₂ independently is C₃-C₆cycloalkyl, or is C₃-C₆cycloalkyl mono-, di- or tri-substituted by halogen, —CN, —CO₂R₁₁₆, —CONR₁₁₇R₁₁₈, —COR₁₁₉, —C(R₁₂₀)═NOR₁₂₁, —C(S)NR₁₂₂R₁₂₃ or by —C(C₁-C₄alkylthio)═NR₁₂₄; or
two adjacent R₂ substituents together form a C₁-C₇alkylene bridge which may be interrupted by from 1 to 3 hetero atoms selected from oxygen, nitrogen and sulfur and may be mono-, di- or tri-substituted by halogen, C₁-C₆alkyl or by C₁-C₆alkoxy, the total number of ring atoms being at least 5 and at most 9; or
two adjacent R₂ substituents together form a C₂-C₇alkenylene bridge which may be interrupted by from 1 to 3 hetero atoms selected from oxygen, nitrogen and sulfur and may be mono-, di- or tri-substituted by halogen, C₁-C₆alkyl or by C₁-C₆alkoxy, the total number of ring atoms being at least 5, and at most 9;
R₇₁ is hydrogen or C₁-C₈alkyl;
R₇₂ is hydrogen, C₁-C₈alkyl, C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₇₁ and R₇₂ together are a C₂-C₅alkylene chain which may be interrupted by an oxygen or sulfur atom;
R₇₃ is hydrogen, C₁-C₈alkyl, C₃-C₈alkenyl or C₃-C₈alkynyl, or is C₁-C₈alkyl, C₃-C₈alkenyl or C₃-C₈alkynyl mono-, di- or tri-substituted by halogen, C₁-C₄alkoxy or by phenyl, it being possible for phenyl in turn to be mono-, di- or tri-substituted by halogen, C₁-C₄alkyl, C₁-C₄-haloalkyl, C₁-C₄alkoxy, —CN, —NO₂, C₁-C₄alkylthio, C₁-C₄alkylsulfinyl or by C₁-C₄alkylsulfonyl;
R₇₄ is hydrogen or C₁-C₈alkyl;
R₇₅ is hydrogen, C₁-C₈alkyl or C₃-C₇cycloalkyl, or is C₁-C₈alkyl mono-, di- or tri-substituted by —COOH, C₁-C₈alkoxycarbonyl, C₁-C₆alkoxy or by —CN; or
R₇₅ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₇₄ and R₇₅ together are a C₂-C₅alkylene chain which may be interrupted by an oxygen or sulfur atom;
R₇₆ is hydrogen, C₁-C₄alkyl, C₁-C₄haloalkyl or C₃-C₆cycloalkyl;
R₇₇ is hydrogen, C₁-C₄alkyl, C₁-C₄haloalkyl or C₃-C₆cycloalkyl;
R₇₈ is hydrogen, C₁-C₈alkyl, C₃-C₈alkenyl, C₃-C₈alkynyl, C₁-C₄haloalkyl or C₃-C₆haloalkenyl; and
R₇₉ is hydrogen or C₁-C₈alkyl;
R₈₀ is hydrogen or C₁-C₈alkyl, or is C₁-C₈alkyl mono-, di- or tri-substituted by —COOH, C₁-C₈alkoxycarbonyl or by —CN, or
R₈₀ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₇₉ and R₈₀ together are C₂-C₅alkylene;
R₈₁ is hydrogen or C₁-C₈alkyl;
R₈₂ is —Si(C₁-C₆alkyl)₃, C₃-C₈alkenyl or C₃-C₈alkynyl, or is C₁-C₈alkyl which is mono-, di- or tri-substituted by halogen, —CN, —NH₂, C₁-C₆alkylamino, di(C₁-C₆alkyl)amino or by C₁-C₄alkoxy;
R₈₃ is hydrogen, C₁-C₈alkyl, C₃-C₈alkenyl or C₃-C₈alkynyl, or is C₁-C₈alkyl which is mono-, di- or tri-substituted by halogen, —CN, —NH₂, C₁-C₆alkylamino, di(C₁-C₆alkyl)amino or by C₁-C₄alkoxy;
R₈₄, R₈₅ and R₈₆ are each independently of the others c₁-C₈alkyl, C₃-C₈alkenyl or C₃-C₈-alkynyl, or C₁-C₈alkyl which is mono-, di- or tri-substituted by halogen, —CN or by C₁-C₄-alkoxy;
R₈₇ and R₈₉ are each independently of the other hydrogen, C₁-C₈alkyl or C₁-C₈alkoxy;
R₈₈ is C₁-C₈alkyl;
R₉₀ is hydrogen or C₁-C₈alkyl;
R₉₁ is C₁-C₄alkyl;
R₉₂ and R₉₃ are each independently of the other C₁-C₆alkyl;
R₉₄ is hydrogen, or is C₁-C₈alkyl, C₃-C₈alkenyl or C₃-C₈alkynyl, each of which may be mono-, di- or tri-substituted by halogen, C₁-C₄alkoxy or by phenyl, it being possible for phenyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl;
R₉₅ is hydrogen or C₁-C₈alkyl;
R₉₆ is hydrogen or C₁-C₈alkyl, or is C₁-C₈alkyl mono-, di- or tri-substituted by —COOH, C₁-C₈alkoxycarbonyl or by —CN; or
R₉₆ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₉₅ and R₉₆ together are C₂-C₅alkylene;
R₉₇ and R₉₈ are each independently of the other hydrogen, C₁-C₄alkyl, C₁-C₄haloalkyl or C₃-C₆cycloalkyl;
R₉₉ is hydrogen, C₁-C₈alkyl, C₃-C₈alkenyl, C₃-C₈alkynyl, C₁-C₄haloalkyl or C₃-C₆haloalkenyl;
R₁₀₀ is hydrogen or C₁-C₈alkyl;
R₁₀₁ is hydrogen or C₁-C₈alkyl, or is C₁-C₈alkyl mono-, di- or tri-substituted by —COOH, C₁-C₈alkoxycarbonyl or by —CN; or
R₁₀₁ is C₃-C₈alkenyl, C₃-C₈alkynyl, phenyl or benzyl, it being possible for phenyl and benzyl in turn to be mono-, di- or tri-substituted by halogen; C₁-C₄alkyl, C₁-C₄haloalkyl, C₁-C₄alkoxy, —CN, —NO₂, C₁-C₄alkylthio; C₁-C₄alkylsulfinyl or by C₁-C₄alkylsulfonyl; or
R₁₀₀ and R₁₀₁ together are C₂-C₅alkylene;
R₁₀₂ is hydrogen or C₁-C₈alkyl;
R₁₀₃ is hydrogen, C₁-C₈alky, —Si(C₁-C₆alkyl)₃, C₃-C₈alkenyl or C₃-C₈alkynyl;
R₁₀₄ is C₁-C₆alkyl;
R₁₀₅ is hydrogen or is C₁-C₈alkyl, C₃-C₈alkenyl or C₃-C₈alkynyl, each of which may be mono-, di- or tri-substituted by halogen, C₁-C₄alkoxy or by phenyl, it being possible for phenyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsufinyl or by C₁-C₄alkylsulfonyl;
R₁₀₆ is hydrogen or C₁-C₈alkyl;
R₁₀₇ is hydrogen or C₁-C₈alkyl, or is C₁-C₈alkyl mono-, di- or tri-substituted by —COOH, C₁-C₈alkoxycarbonyl or by —CN, or
R₁₀₇ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₀₆ and R₁₀₇ together are C₂-C₅alkylene;
R₁₀₈ is hydrogen, C₁-C₄alkyl, C₁-C₄haloalkyl or C₃-C₆cycloalkyl;
R₁₀₉ is hydrogen, C₁-C₄alkyl, C₁-C₄haloalkyl or C₃-C₆cycloalkyl;
R₁₁₀ is hydrogen, C₁-C₈alkyl, C₃-C₈alkenyl, C₃-C₈alkynyl, C₁-C₄haloalkyl or C₃-C₆haloalkenyl;
R₁₁₁ is hydrogen or C₁-C₈alkyl;
R₁₁₂ is hydrogen or C₁-C₈alkyl, or is C₁-C₈alkyl mono-, di- or tri-substituted by —COOH, C₁-C₈alkoxycarbonyl or by —CN; or
R₁₁₂ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₁₁ and R₁₁₂ together are C₂-C₅alkylene;
R₁₁₃ is hydrogen or C₁-C₈alkyl;
R₁₁₄ is hydrogen, C₁-C₈alkyl, —Si(C₁-C₆alkyl)₃, C₃-C₈alkenyl or C₃-C₈alkynyl;
R₁₁₅ is C₁-C₆alkyl;
R₁₁₆ is hydrogen or is C₁-C₈alkyl, C₃-C₈alkenyl or C₃-C₈alkynyl, each of which may be mono-, di- or tri-substituted by halogen, C₁-C₄alkoxy or by phenyl, it being possible for phenyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl;
R₁₁₇ is hydrogen or C₁-C₈alky;
R₁₁₈ is hydrogen or C₁-C₈alkyl, or is C₁-C₈alkyl mono-, di- or tri-substituted by —COOH, C₁-C₈alkoxycarbonyl or by —CN; or
R₁₁₈ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₁₇ and R₁₁₈ together are C₂-C₅alkylene;
R₁₁₉ is hydrogen, C₁-C₄alkyl, C₁-C₄haloalkyl or C₃-C₆cycloalkyl;
R₁₂₀ is hydrogen, C₁-C₄alkyl, C₁-C₄haloalkyl or C₃-C₆cycloalkyl;
R₁₂₁ is hydrogen, C₁-C₈alkyl, C₃-C₈alkenyl, C₃-C₈alkynyl, C₁-C₄haloalkyl or C₃-C₆haloalkenyl;
R₁₂₂ is hydrogen or C₁-C₈alkyl;
R₁₂₃ is hydrogen or C₁-C₈alkyl, or is C₁-C₈alkyl mono-, di- or tri-substituted by —COOH, C₁-C₈alkoxycarbonyl or by —CN; or
R₁₂₃ is C₃-C₈alkenyl, C₃-C₈alkynyl, phenyl or benzyl, it being possible for phenyl and benzyl in turn to be mono-, di- or tri-substituted by halogen, C₁-C₄alkyl, C₁-C₄haloalkyl, C₁-C₄alkoxy, —CN, —NO₂, C₁-C₄alkylthio, C₁-C₄alkylsulfinyl or by C₁-C₄alkylsulfonyl; or
R₁₂₂ and R₁₂₃ together are C₂-C₅alkylene; and
R₁₂₄ is hydrogen or C₁-C₈alkyl.

3. A compound according to claim 2, wherein each R₁ independently is halogen, —CN, —NO₂, —C(R₁₀)═NOR₁₁, —OR₁₃, —SO₂R₁₆, —OSO₂R₁₇, C₁-C₈alkyl or C₂-C₈alkenyl, or is C₁-C₈alkyl mono-, di- or tri-substituted by halogen or by —CN; R₁₀ is hydrogen or C₁-C₄alkyl; and R₁₁ is C₁-C₈alkyl.

4. A compound according to claim 2, wherein Q is a group Q₁, Q₂, Q₃ or Q₅.

5. A compound according to claim 4, wherein Q is a group Q₁ or Q₂.

6. A compound according to claim 2, wherein each R₂ independently is halogen, —CN, —SCN, —OCN, —N₃, —CONR₃₆R₃₇, —C(R₃₈)═NOR₃₉, —COR₄₀, —OR₄₁, —OSO₂R₄₅, —N([CO]ₚR₄₆)COR₄₇, —N(R₅₆)SO₂R₅₇, —N(SO₂R₅₈)SO₂R₅₉, —N═C(OR₆₀)R₆₁ or C₁-C₈alkyl, or is C₁-C₈alkyl mono-, di- or tri-substituted by halogen, —CN, —N₃, —SCN, —CONR₇₄R₇₅, —COR₇₆, —C(R₇₇)═NOR₇₈, —C(S)NR₇₉R₈₀, —OR₈₂, —SOR₈₄, —SO₂R₈₅ or by —N(R₈₉)COR₉₀.

7. A process for the preparation of a compound of formula Ia 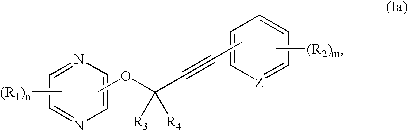
wherein R₁, R₂, R₃, R₄, Z, m and n are as defined in claim 1, which process comprises reacting a compound of formula IIa 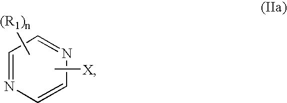
wherein R₁ and n are as defined in claim 1 (Q=Q₁) and X is halogen, with a compound of formula IIIa 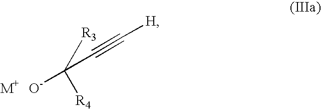
wherein R₃ and R₄ are as defined in claim 1 and M⁺ is an alkali metal cation, to form a compound of formula IVa 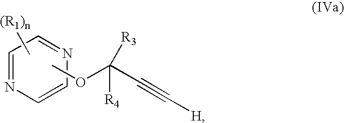
wherein R₁, R₃, R₄ and n are as defined in claim 1, and then coupling that compound with a compound of formula V 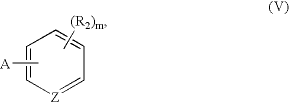
wherein Z, R₂ and m are as defined in claim 1 and A is a leaving group, in the presence of a palladium catalyst.

8. A herbicidal and plant-growth-inhibiting composition which comprises on an inert carrier, a herbicidally effective amount of a compound of formula I.

9. A method of controlling undesired plant growth, which comprises applying a herbicidally effective amount of a compound of formula I or of a composition comprising such a compound to the plants or to the locus thereof.

10. A method of inhibiting plant growth, which comprises applying a herbicidally effective amount of a compound of formula I or of a composition comprising such a compound to the plants or to the locus thereof.
